(19) United States
(12) Patent Application Publication
(10) Pub. No.: US 2024/0209442 A1
(43) Pub. Date: Jun. 27, 2024

(54) METHODS AND SYSTEMS FOR ANALYZING COMPLEX GENOMIC REGIONS

(71) Applicant: RPRD Diagnostics, LLC, Wauwatosa, WI (US)

(72) Inventor: Gunter SCHARER, Wauwatosa, WI (US)

(21) Appl. No.: 18/554,174

(22) PCT Filed: Apr. 5, 2022
(86) PCT No.: PCT/US2022/023483

§ 371 Date: Oct. 5, 2023

Related U.S. Application Data
(60) Provisional application No. 63/171,387, filed on Apr. 6, 2021.

(51) Int. Cl. C12Q 1/6876; C12Q 1/6806; C12Q 1/683; C12Q 1/6874

(52) U.S. Cl. CPC C12Q 1/6876; C12Q 1/683; C12Q 1/6874; C12Q 1/6806; C12Q 2600/156

(57) ABSTRACT

Provided herein are improved methods of analyzing (e.g., sequencing, genotyping, structural analysis) complex genomic regions. In some cases, the methods involve the use of a CRISPR-associated endonuclease and an outer pair of guide RNAs and an inner pair of guide RNAs to excise a genomic region of interest from genomic DNA. The methods further involve the use of long-read sequencing to sequence the genetic region of interest. In some cases, the methods are amplification-free.

### CROSS-REFERENCE

This application claims the benefit of U.S. Provisional Application No. 63/171,387, filed Apr. 6, 2021, which application is incorporated herein by reference in its entirety.

### SEQUENCE LISTING

The instant application contains a Sequence Listing which has been submitted electronically in ASCII format and is hereby incorporated by reference in its entirety. Said ASCII copy, created on Apr. 5, 2022, is named 57312-702_601_SL.txt and is 109,652 bytes in size.

### BACKGROUND

As genetic variation can influence the response to a medication, pharmacogenetics (PGx) represents a component of precision medicine that enables individualized determination of drug response. The benefits of PGx include reduced cost and risk of adverse drug reactions (SADRs), as well as improved drug efficacy. While there is a large number of PGx genes currently tested, Cytochrome P450 2D6 (CYP2D6) is of tremendous diagnostic value, as up to 25% of all drugs are activated or metabolized by CYP2D6. These drugs include cancer drugs, opioid agonists, and several antidepressants and antianxiety medications. The CYP2D6 enzyme is encoded by the CYP2D6 gene and genetic variation can cause a reduction or complete loss of enzyme function. CYP2D6 is primarily expressed in the liver and is a major contributor to hepatic drug metabolism and clearance. Problems with correctly diagnosing CYP2D6 genetic variation can directly affect the risk for the development of SADRs. The NIH Clinical Pharmacogenetics Implementation Consortium (CPIC) currently lists 58 drugs associated with evidence supporting clinical testing of CYP2D6, thereby making it one of the top genes. In the US alone, CYP2D6 testing is estimated to be a $522M market in 2019 with an annual growth rate of 6-8%.

At this time, there are over 100 described pharmacogenetic relevant alterations (also called *star allele haplo-types) in CYP2D6, including frequent copy number variation. In addition, gene-fusions and hybrids with neighboring highly homologous (up to 94% identical) pseudogenes (CYP2D7 and CYP2D8) complicate variant calling. In the United States ˜13% of people carry a CYP2D6 structural variant and these variants represent 7% of all variation associated with the gene. These features complicate genetic analysis with current testing platforms and many of the rare or more complex haplotypes are not accurately analyzed. Work from many groups have demonstrated that currently used commercial genotyping platforms are prone to mischaracterize CYP2D6. This leads to incorrect assignment, which results in incorrect dosing recommendations. Gene sequencing is similarly hampered when based on short reads (NGS) or template length (Sanger sequencing). While a number of methods have been developed which combine targeted amplification, copy number analysis, and long-range PCR to more precisely determine the full structure, these methods are not suitable for routine clinical testing due to the complex workflow, time requirements, and overall cost.

### SUMMARY

There is an unmet need for improved methods and systems for accurately and cost-effectively analyzing complex genomic regions. This disclosure meets this unmet need.

In one aspect of the disclosure, a method of analyzing (e.g., sequencing, genotyping, structural analysis) a genomic region of interest is provided, the method comprising: a) contacting genomic DNA comprising the genomic region of interest with a Clustered Regularly Interspaced Short Palindromic Repeat (CRISPR)-associated endonuclease and an outer pair of guide RNAs (gRNAs), thereby generating a first excised fragment comprising the genomic region of interest; b) contacting the first excised fragment with a CRISPR-associated endonuclease and an inner pair of gRNAs, thereby generating a second excised fragment comprising the genomic region of interest; and c) analyzing the genomic region of interest contained within the second excised fragment. In some cases, the CRISPR-associated endonuclease and the outer pair of gRNAs of a) associate with and block the 5′ and 3′ ends of the first excised fragment. In some cases, the method further comprises, prior to b), contacting the product of a) with one or more exonucleases, such that background genomic DNA is digested and the first excised fragment is not digested. In some cases, the one or more exonucleases are selected from the group consisting of: exonuclease I, exonuclease II, exonuclease III, exonuclease IV, exonuclease V, exonuclease VI, exonuclease VII, exonuclease VIII, and any combination thereof. In some cases, the outer pair of gRNAs comprises a first outer gRNA and a second outer gRNA. In some cases, the first outer gRNA comprises a nucleotide sequence that is substantially complementary to a first nucleotide sequence present in the genomic DNA, and the second outer gRNA comprises a nucleotide sequence that is substantially complementary to a second nucleotide sequence present in the genomic DNA. In some cases, the first nucleotide sequence and the second nucleotide sequence are different. In some cases, the first nucleotide sequence and the second nucleotide sequence flank the genomic region of interest. In some cases, the first nucleotide sequence, the second nucleotide sequence, or both, are present in the genomic DNA up to about 100 kilobases in length from the genomic region of interest. In some cases, the inner pair of gRNAs comprises a first inner gRNA and a second inner gRNA. In some cases, the first inner gRNA comprises a nucleotide sequence that is substantially complementary to a third nucleotide sequence present in the genomic DNA, and the second inner gRNA comprises a nucleotide sequence that is substantially complementary to a fourth nucleotide sequence present in the genomic DNA. In some cases, the third nucleotide sequence and the fourth nucleotide sequence are different. In some cases, the third nucleotide sequence and the fourth nucleotide sequence flank the genomic region of interest. In some cases, the third nucleotide sequence and the fourth nucleotide sequence are present on the genomic DNA at a base length closer to the genomic region of interest than the first nucleotide sequence and the second nucleotide sequence. In some cases, the second excised fragment is smaller in base length than the first excised fragment. In some cases, the analyzing comprises sequencing the genomic region of interest contained within the second excised fragment. In some cases, the genomic DNA is provided at an amount of about 10 μg or greater. In some cases, the analyzing comprises genotyping the genomic region of interest contained within the second excised fragment. In some cases, the analyzing comprises performing structural analysis on the genomic region of interest contained within the second excised fragment. In some cases, the method further comprises, prior to b), isolating the first excised fragment. In some cases, the method further comprises, prior to c), isolating the second excised fragment. In some cases, the method does not involve DNA amplification. In some cases, the method further comprises, prior to c), attaching one or more adapters to the 5′ end, the 3′ end, or both, of the second excised fragment. In some cases, the CRISPR-associated endonuclease is a Class 1 CRISPR-associated endonuclease or a Class 2 CRISPR-associated endonuclease. In some cases, the Class 1 CRISPR-associated endonuclease is selected from the group consisting of: Cas3, Cas5, Cas8a, Cas8b, Cas8c, Cas10d, Cse1, Cse2, Csy1, Csy2, Csy3, GSU0054, Cas10, Csm2, Cmr5, Csx11, Csx10, and Csf1. In some cases, the Class 2 CRISPR-associated endonuclease is selected from the group consisting of: Cas9, Cas12a, Csn2, Cas4, Cas12b, Cas12c, Cas13a, Cas13b, Cas13c, and Cas13d. In some cases, the CRISPR-associated endonuclease comprises an amino acid sequence having at least 80% sequence identity to a wild-type CRISPR-associated endonuclease. In some cases, the CRISPR-associated endonuclease is Cas9 or a variant thereof. In some cases, the Cas9 is a Streptococcus pyogenes Cas9 (spCas9). In some cases, the Cas9 variant comprises one or more point mutations, relative to a wild-type Streptococcus pyogenes Cas9 (spCas9), selected from the group consisting of: R780A, K810A, K848A, K855A, H982A, K1003A, R1060A, D1135E, N497A, R661A, Q695A, Q926A, L169A, Y450A, M495A, M694A, and M698A. In some cases, the genomic DNA is not fragmented, digested, or sheared prior to a). In some cases, the genomic DNA is not subjected to restriction enzyme digestion prior to a). In some cases, the genomic region of interest is a complex genomic region. In some cases, the complex genomic region comprises a gene of interest and one or more pseudogenes thereof. In some cases, the one or more pseudogenes comprise a nucleotide sequence having at least 75% sequence identity to the gene of interest. In some cases, the complex genomic region comprises one or more repetitive regions, one or more duplications, one or more insertions, one or more inversions, one or more tandem repeats, one or more retrotransposons, or any combination thereof. In some cases, the genomic region of interest is a highly polymorphic gene locus. In some cases, the first excised fragment is at least about 0.06 kilobases in length. In some cases, the first excised fragment is up to about 200 kilobases in length. In some cases, the second excised fragment is at least about 0.02 kilobases in length. In some cases, the second excised fragment is up to about 199.98 kilobases in length. In some cases, the sequencing comprises long-read sequencing. In some cases, the long-read sequencing comprises single-molecule real-time sequencing or nanopore sequencing. In some cases, the method does not involve any one of polymerase chain reaction (PCR) or isothermal amplification. In some cases, the method does not involve any one of multiple displacement amplification (MDA), strand displacement amplification (SDA), nucleic acid sequence based amplification (NASBA), loop-mediated isothermal amplification, rolling circle amplification (RCA), ligase chain reaction (LCR), helicase dependent amplification, or ramification amplification method. In some cases, the genomic DNA is provided or obtained in a biological sample. In some cases, the biological sample comprises a body fluid (e.g., blood (e.g., whole blood, plasma, serum), urine, saliva, bone marrow, spinal fluid, sputum, ascites, lymphatic fluid, pleural fluid, amniotic fluid, semen, vaginal fluid, sweat, stool, glandular secretions, ocular fluids, breast milk) or a solid tissue sample. In some cases, the biological sample is a diagnostic sample. In some cases, the genomic region of interest is a genetic locus comprising CYP2D6, CYP2D7, and CYP2D8. In some cases, the analyzing comprises identifying one or more genetic variations in CYP2D6. In some cases, the method further comprises, identifying a subject as having a reduction, a loss of, or an increase in CYP2D6 function based on the genetic variation. In some cases, the method further comprises, recommending a treatment or an alternative treatment to the subject based on the identifying. In some cases, the subject is identified as having a reduction in, a loss of, or an increase in CYP2D6 function, recommending an alternative treatment to the subject. In some cases, the method further comprises, recommending a dosage of a therapeutic to the subject based on the identifying. In some cases, when the subject is identified as having a reduction in, a loss of, or an increase in CYP2D6 function, altering a dosage of a therapeutic. In some cases, the outer pair of gRNAs, the inner pair of gRNAs, or both, are selected from any one of SEQ ID NOS: 1-418.

In another aspect, a kit for analyzing a genomic region of interest is provided, the kit comprising: a) a Clustered Regularly Interspaced Short Palindromic Repeat (CRISPR)-associated endonuclease; b) an outer pair of gRNAs comprising: i) a first outer gRNA comprising a nucleotide sequence that is substantially complementary to a first nucleotide sequence present in genomic DNA that is upstream of the genomic region of interest; and ii) a second outer gRNA comprising a nucleotide sequence that is substantially complementary to a second nucleotide sequence present in genomic DNA that is downstream of the genomic region of interest; c) an inner pair of gRNAs comprising: iii) a first inner gRNA comprising a nucleotide sequence that is substantially complementary to a third nucleotide sequence present in genomic DNA that is upstream of the genomic region of interest; and iv) a second inner gRNA comprising a nucleotide sequence that is substantially complementary to a fourth nucleotide sequence present in genomic DNA that is downstream of the genomic region of interest, wherein the third nucleotide sequence and the fourth nucleotide sequence are present on the genomic DNA at a base length closer to the genomic region of interest than the first nucleotide sequence and the second nucleotide sequence. In some cases, the kit further comprises, one or more exonucleases. In some cases, the one or more exonucleases are selected from the group consisting of: exonuclease I, exonuclease II, exonuclease III, exonuclease IV, exonuclease V, exonuclease VI, exonuclease VII, exonuclease VIII, and any combination thereof. In some cases, the CRISPR-associated endonuclease is a Class 1 or a Class 2 CRISPR-associated endonuclease. In some cases, the Class 1 CRISPR-associated endonuclease is selected from the group consisting of: Cas3, Cas5, Cas8a, Cas8b, Cas8c, Cas10d, Cse1, Cse2, Csy1, Csy2, Csy3, GSU0054, Cas10, Csm2, Cmr5, Csx11, Csx10, and Csf1. In some cases, the Class 2 CRISPR-associated endonuclease is selected from the group consisting of: Cas9, Cas12a, Csn2, Cas4, Cas12b, Cas12c, Cas13a, Cas13b, Cas13c, and Cas13d. In some cases, the CRISPR-associated endonuclease comprises an amino acid sequence having at least 80% sequence identity to a wild-type CRISPR-associated endonuclease. In some cases, the CRISPR-associated endonuclease is Cas9 or a variant thereof. In some cases, the Cas9 is a Streptococcus pyogenes Cas9 (spCas9). In some cases, the Cas9 variant comprises one or more point mutations, relative to a wild-type Streptococcus pyogenes Cas9 (spCas9), selected from the group consisting of: R780A, K810A, K848A, K855A, H982A, K1003A, R1060A, D1135E, N497A, R661A, Q695A, Q926A, L169A, Y450A, M495A, M694A, and M698A. In some cases, the genomic region of interest is a genomic locus comprising CYP2D6, CYP2D7, and CYP2D8. In some cases, the first outer guide RNA, the first inner guide RNA, or both, comprise the nucleotide sequence of any one of SEQ ID NOS: 3-12, 17-26, 68-77, 82-214, and 344-418. In some cases, the second outer guide RNA, the second inner guide RNA, or both, comprise the nucleotide sequence of any one of SEQ ID NOS: 1, 2, 13-16, 27-67, 78-81, and 215-343. In some cases, the kit further comprises, instructions for using the kit in a nested CRISPR reaction. In some cases, the kit further comprises, instructions for using the kit to excise the genomic region of interest from genomic DNA.

In one aspect, a method of analyzing a genomic region of interest is provided, the method comprising: (a) contacting genomic DNA comprising the genomic region of interest with a Clustered Regularly Interspaced Short Palindromic Repeat (CRISPR)-associated endonuclease and two or more gRNAs, thereby generating an excised genomic region of interest; (b) isolating the genomic DNA comprising the genomic region of interest; and (c) analyzing the excised genomic region of interest, wherein the method does not involve DNA amplification. In some cases, the analyzing comprises sequencing the excised genomic region of interest. In some cases, the analyzing comprises genotyping the excised genomic region of interest. In some cases, the analyzing comprises performing structural analysis on the excised region of interest. In some cases, the isolating of (b) is performed prior to the contacting of (a). In some cases, the isolating of (b) is performed after the contacting of (a). In some cases, the two or more gRNAs each comprise a nucleotide sequence that is substantially complementary to different nucleotide sequences present in the genomic DNA. In some cases, the different nucleotide sequences flank the genomic region of interest. In some cases, the CRISPR-associated endonuclease cleaves the genomic region of interest at genomic sites flanking the genomic region of interest. In some cases, the CRISPR-associated endonuclease is a Class 1 or a Class 2 CRISPR-associated endonuclease. In some cases, the Class 1 CRISPR-associated endonuclease is selected from the group consisting of: Cas3, Cas5, Cas8a, Cas8b, Cas8c, Cas10d, Cse1, Cse2, Csy1, Csy2, Csy3, GSU0054, Cas10, Csm2, Cmr5, Csx11, Csx10, and Csf1. In some cases, the Class 2 CRISPR-associated endonuclease is selected from the group consisting of: Cas9, Cas12a, Csn2, Cas4, Cas12b, Cas12c, Cas13a, Cas13b, Cas13c, and Cas13d. In some cases, the CRISPR-associated endonuclease comprises an amino acid sequence having at least 80% sequence identity to a wild-type CRISPR-associated endonuclease. In some cases, the CRISPR-associated endonuclease is Cas9 or a variant thereof. In some cases, the Cas9 is a Streptococcus pyogenes Cas9 (spCas9). In some cases, the Cas9 variant comprises one or more point mutations, relative to a wild-type Streptococcus pyogenes Cas9 (spCas9), selected from the group consisting of: R780A, K810A, K848A, K855A, H982A, K1003A, R1060A, D1135E, N497A, R661A, Q695A, Q926A, L169A, Y450A, M495A, M694A, and M698A. In some cases, the genomic DNA is not fragmented, digested, or sheared prior to (a). In some cases, the genomic DNA is not subjected to restriction enzyme digestion prior to (a). In some cases, the genomic region of interest is a complex genomic region. In some cases, the complex genomic region comprises a gene and one or more pseudogenes thereof. In some cases, the one or more pseudogenes comprise a nucleotide sequence having at least 75% sequence identity to the gene. In some cases, the complex genomic region comprises one or more repetitive regions, one or more duplications, one or more insertions, one or more inversions, one or more tandem repeats, one or more retrotransposons, or any combination thereof. In some cases, the genomic region of interest is a highly polymorphic gene locus. In some cases, the excised genomic region of interest is at least 10 kilobases in length. In some cases, the excised genomic region of interest is up to 250 kilobases in length. In some cases, the isolating comprises isolating high molecular weight DNA. In some cases, the high molecular weight DNA is at least 50 kilobases in length. In some cases, the sequencing comprises long-read sequencing. In some cases, the long-read sequencing comprises single-molecule real-time sequencing or nanopore sequencing. In some cases, the method further comprises, ligating one or more sequencing adapters to one or both ends of the excised genomic region of interest. In some cases, the method further comprises, prior to a), dephosphorylating the genomic DNA. In some cases, the dephosphorylating comprises treating the genomic DNA with a phosphatase. In some cases, the phosphatase is shrimp alkaline phosphatase. In some cases, the method further comprises, after the dephosphorylating, treating the genomic DNA with Terminal Transferase (TdT). In some cases, the method further comprises, end-tailing the excised genomic region of interest. In some cases, the end-tailing comprises adding one or more adenosine nucleotides to a free 3′ end of the excised genomic region of interest. In some cases, the method does not involve any one of polymerase chain reaction (PCR) or isothermal amplification. In some cases, the method does not involve any one of multiple displacement amplification (MDA), strand displacement amplification (SDA), nucleic acid sequence based amplification (NASBA), loop-mediated isothermal amplification, rolling circle amplification (RCA), ligase chain reaction (LCR), helicase dependent amplification, or ramification amplification method. In some cases, the genomic DNA is provided in a biological sample. In some cases, the biological sample comprises a body fluid (e.g., blood (e.g., whole blood, plasma, serum), urine, saliva, bone marrow, spinal fluid, sputum, ascites, lymphatic fluid, pleural fluid, amniotic fluid, semen, vaginal fluid, sweat, stool, glandular secretions, ocular fluids, breast milk) or a solid tissue sample. In some cases, the biological sample is a diagnostic sample.

In another aspect, a method of analyzing a complex genomic region of interest of at least 10 kilobases in length is provided, the method comprising: (a) providing genomic DNA comprising the complex genomic region of interest; (b) isolating high-molecular weight DNA comprising the complex genomic region of interest; (c) contacting the genomic DNA with a Clustered Regularly Interspaced Short Palindromic Repeat (CRISPR)-associated endonuclease and two or more gRNAs to excise the complex genomic region of interest, wherein the two or more gRNAs each comprise nucleotide sequences substantially complementary to different nucleotide sequences present in the genomic DNA, and wherein the different nucleotide sequences flank the complex genomic region of interest; and (d) analyzing the complex genomic region of interest, wherein the method does not involve DNA amplification. In some cases, the analyzing comprises sequencing the complex genomic region of interest. In some cases, the sequencing comprises long-read sequencing. In some cases, the long-read sequencing comprises single-molecule real-time sequencing or nanopore sequencing. In some cases, the analyzing comprises genotyping the complex genomic region of interest. In some cases, the analyzing comprises performing structural analysis of the genomic region of interest. In some cases, the isolating of (b) is performed prior to the contacting of (c). In some cases, the isolating of (b) is performed after the contacting of (c). In some cases, the high-molecular weight DNA is at least 10 kilobases in length. In some cases, the complex genomic region of interest comprises a target gene and one or more pseudogenes thereof. In some cases, the one or more pseudogenes have at least 75% sequence identity to the target gene. In some cases, the complex genomic region of interest comprises CYP2D6, CYP2D7, and CYP2D8. In some cases, the complex genomic region of interest comprises CYP2C8, CYP2C9, CYP2C18, and CYP2C19. In some cases, the complex genomic region of interest comprises one or more repetitive regions, one or more duplications, one or more insertions, one or more inversions, one or more tandem repeats, one or more retrotransposons, or any combination thereof. In some cases, the complex genomic region of interest is a highly polymorphic gene locus. In some cases, the CRISPR-associated endonuclease is a Class 1 or a Class 2 CRISPR-associated endonuclease. In some cases, the Class 1 CRISPR-associated endonuclease is selected from the group consisting of: Cas3, Cas5, Cas8a, Cas8b, Cas8c, Cas10d, Cse1, Cse2, Csy1, Csy2, Csy3, GSU0054, Cas10, Csm2, Cmr5, Csx11, Csx10, and Csf1. In some cases, the Class 2 CRISPR-associated endonuclease is selected from the group consisting of: Cas9, Cas12a, Csn2, Cas4, Cas12b, Cas12c, Cas13a, Cas13b, Cas13c, and Cas13d. In some cases, the CRISPR-associated endonuclease comprises an amino acid sequence having at least 80% sequence identity to a wild-type CRISPR-associated endonuclease. In some cases, the CRISPR-associated endonuclease is Cas9 or a variant thereof. In some cases, the Cas9 is a Streptococcus pyogenes Cas9 (spCas9). In some cases, the Cas9 variant comprises one or more point mutations, relative to a wild-type Streptococcus pyogenes Cas9 (spCas9), selected from the group consisting of: R780A, K810A, K848A, K855A, H982A, K1003A, R1060A, D1135E, N497A, R661A, Q695A, Q926A, L169A, Y450A, M495A, M694A, and M698A. In some cases, the genomic DNA is not fragmented or digested prior to a). In some cases, the genomic DNA is not subjected to restriction enzyme digestion prior to a). In some cases, the complex genomic region of interest is up to 250 kilobases in length. In some cases, the method further comprises, ligating one or more sequencing adapters to one or both ends of the excised genomic region of interest. In some cases, the method does not involve any one of polymerase chain reaction (PCR) or isothermal amplification. In some cases, the method does not involve any one of multiple displacement amplification (MDA), strand displacement amplification (SDA), nucleic acid sequence based amplification (NASBA), loop-mediated isothermal amplification, rolling circle amplification (RCA), ligase chain reaction (LCR), helicase dependent amplification, or ramification amplification method. In some cases, the genomic DNA is provided in a biological sample. In some cases, the biological sample is a body fluid (e.g., blood (e.g., whole blood, plasma, serum), urine, saliva, bone marrow, spinal fluid, sputum, ascites, lymphatic fluid, pleural fluid, amniotic fluid, semen, vaginal fluid, sweat, stool, glandular secretions, ocular fluids, breast milk) or a solid tissue sample. In some cases, the biological sample is a diagnostic sample.

In another aspect, a method of analyzing a genetic locus comprising CYP2D6, CYP2D7, and CYP2D8 is provided, the method comprising: (a) providing genomic DNA comprising the genetic locus; (b) contacting the genomic DNA with a Clustered Regularly Interspaced Short Palindromic Repeat (CRISPR)-associated endonuclease and two or more gRNAs to excise the genetic locus from the genomic DNA, wherein the two or more gRNAs each comprise nucleotide sequences substantially complementary to different nucleotide sequences present in the genomic DNA, and wherein the different nucleotide sequences flank the genetic locus comprising CYP2D6, CYP2D7, and CYP2D8; and (c) analyzing the genetic locus. In some cases, the analyzing comprises sequencing the genetic locus. In some cases, the sequencing comprises long-read sequencing. In some cases, the long-read sequencing comprises single-molecule real-time sequencing or nanopore sequencing. In some cases, the analyzing comprises genotyping the genetic locus. In some cases, the analyzing comprises performing structural analysis of the genetic locus. In some cases, the method further comprises, prior to c), isolating high molecular weight DNA comprising the genetic locus. In some cases, the high molecular weight DNA is at least 10 kilobases in length. In some cases, the two or more gRNAs comprise a nucleotide sequence selected from the group consisting of: SEQ ID NOS: 1-418. In some cases, the genetic locus is at least 40 kilobases in length. In some cases, the CRISPR-associated endonuclease is a Class 1 or a Class 2 CRISPR-associated endonuclease. In some cases, the Class 1 CRISPR-associated endonuclease is selected from the group consisting of: Cas3, Cas5, Cas8a, Cas8b, Cas8c, Cas10d, Cse1, Cse2, Csy1, Csy2, Csy3, GSU0054, Cas10, Csm2, Cmr5, Csx11, Csx10, and Csf1. In some cases, the Class 2 CRISPR-associated endonuclease is selected from the group consisting of: Cas9, Cas12a, Csn2, Cas4, Cas12b, Cas12c, Cas13a, Cas13b, Cas13c, and Cas13d. In some cases, the CRISPR-associated endonuclease comprises an amino acid sequence having at least 80% sequence identity to a wild-type CRISPR-associated endonuclease. In some cases, the CRISPR-associated endonuclease is Cas9 or a variant thereof. In some cases, the Cas9 is a Streptococcus pyogenes Cas9 (spCas9). In some cases, the Cas9 variant comprises one or more point mutations, relative to a wild-type Streptococcus pyogenes Cas9 (spCas9), selected from the group consisting of: R780A, K810A, K848A, K855A, H982A, K1003A, R1060A, D1135E, N497A, R661A, Q695A, Q926A, L169A, Y450A, M495A, M694A, and M698A. In some cases, the genomic DNA is not fragmented, digested, or sheared prior to a). In some cases, the genomic DNA is not subjected to restriction enzyme digestion prior to a). In some cases, the method further comprises, ligating one or more sequencing adapters to one or both ends of the excised genetic locus. In some cases, the method does not involve DNA amplification. In some cases, the method does not involve any one of polymerase chain reaction (PCR) or isothermal amplification. In some cases, the method does not involve any one of multiple displacement amplification (MDA), strand displacement amplification (SDA), nucleic acid sequence based amplification (NASBA), loop-mediated isothermal amplification, rolling circle amplification (RCA), ligase chain reaction (LCR), helicase dependent amplification, or ramification amplification method. In some cases, the genomic DNA is provided in a biological sample. In some cases, the biological sample is a body fluid (e.g., blood (e.g., whole blood, plasma, serum), urine, saliva, bone marrow, spinal fluid, sputum, ascites, lymphatic fluid, pleural fluid, amniotic fluid, semen, vaginal fluid, sweat, stool, glandular secretions, ocular fluids, breast milk) or a solid tissue sample. In some cases, the biological sample is a diagnostic sample.

In yet another aspect, a method of identifying genetic variation in CYP2D6 in a subject is provided, the method comprising: (a) providing a biological sample comprising genomic DNA obtained from the subject; (b) contacting the genomic DNA with a Clustered Regularly Interspaced Short Palindromic Repeat (CRISPR)-associated endonuclease and two or more gRNAs to excise a genetic locus comprising CYP2D6, CYP2D7, and CYP2D8; (c) performing long-read sequencing of the genetic locus; and (d) identifying one or more genetic variations in CYP2D6 of the subject. In some cases, the method further comprises, identifying the subject as having a reduction, a loss of, or an increase in CYP2D6 function based on the genetic variation. In some cases, the method further comprises, recommending a treatment or an alternative treatment to the subject based on the identifying. In some cases, when the subject is identified as having a reduction in, a loss of, or an increase in CYP2D6 function, the method further comprises, recommending an alternative treatment to the subject. In some cases, the method further comprises, recommending a dosage of a therapeutic to the subject based on the identifying. In some cases, when the subject is identified as having a reduction in, a loss of, or an increase in CYP2D6 function, the method further comprises, altering a dosage of a therapeutic. In some cases, the method further comprises, prior to c), isolating high molecular weight DNA comprising the genetic locus. In some cases, the high molecular weight DNA is at least 40 kilobases in length. In some cases, the two or more gRNAs each comprise nucleotide sequences substantially complementary to different nucleotide sequences present in the genomic DNA, and wherein the different nucleotide sequences flank the genetic locus comprising CYP2D6, CYP2D7, and CYP2D8. In some cases, the two or more gRNAs comprise a nucleotide sequence selected from the group consisting of: SEQ ID NOS: 1-418. In some cases, the genetic locus is at least 40 kilobases in length. In some cases, the long-read sequencing comprises single-molecule real-time sequencing or nanopore sequencing. In some cases, the CRISPR-associated endonuclease is a Class 1 or a Class 2 CRISPR-associated endonuclease. In some cases, the Class 1 CRISPR-associated endonuclease is selected from the group consisting of: Cas3, Cas5, Cas8a, Cas8b, Cas8c, Cas10d, Cse1, Cse2, Csy1, Csy2, Csy3, GSU0054, Cas10, Csm2, Cmr5, Csx11, Csx10, and Csf1. In some cases, the Class 2 CRISPR-associated endonuclease is selected from the group consisting of: Cas9, Cas12a, Csn2, Cas4, Cas12b, Cas12c, Cas13a, Cas13b, Cas13c, and Cas13d. In some cases, the CRISPR-associated endonuclease comprises an amino acid sequence having at least 80% sequence identity to a wild-type CRISPR-associated endonuclease. In some cases, the CRISPR-associated endonuclease is Cas9 or a variant thereof. In some cases, the Cas9 is a Streptococcus pyogenes Cas9 (spCas9). In some cases, the Cas9 variant comprises one or more point mutations, relative to a wild-type Streptococcus pyogenes Cas9 (spCas9), selected from the group consisting of: R780A, K810A, K848A, K855A, H982A, K1003A, R1060A, D1135E, N497A, R661A, Q695A, Q926A, L169A, Y450A, M495A, M694A, and M698A. In some cases, the genomic DNA is not fragmented, digested, or sheared prior to (a). In some cases, the genomic DNA is not subjected to restriction enzyme digestion prior to (a). In some cases, the method further comprises, ligating one or more sequencing adapters to one or both ends of the excised genomic region of interest. In some cases, the method does not involve DNA amplification. In some cases, the does not involve any one of polymerase chain reaction (PCR) or isothermal amplification. In some cases, the method does not involve any one of multiple displacement amplification (MDA), strand displacement amplification (SDA), nucleic acid sequence based amplification (NASBA), loop-mediated isothermal amplification, rolling circle amplification (RCA), ligase chain reaction (LCR), helicase dependent amplification, or ramification amplification method. In some cases, the biological sample is a body fluid (e.g., blood (e.g., whole blood, plasma, serum), urine, saliva, bone marrow, spinal fluid, sputum, ascites, lymphatic fluid, pleural fluid, amniotic fluid, semen, vaginal fluid, sweat, stool, glandular secretions, ocular fluids, breast milk) or a solid tissue sample.

In yet another aspect, a composition is provided comprising: (a) a Clustered Regularly Interspaced Short Palindromic Repeat (CRISPR)-associated endonuclease; (b) a first guide RNA (gRNA) comprising a nucleotide sequence substantially complementary to a nucleotide sequence present in genomic DNA that is upstream of a genetic locus comprising CYP2D6, CYP2D7, and CYP2D8; and (c) a second guide RNA (gRNA) comprising a nucleotide sequence substantially complementary to a nucleotide sequence present in genomic DNA that is downstream of the genetic locus comprising CYP2D6, CYP2D7, and CYP2D8. In some cases, the first guide RNA comprises a nucleotide sequence selected from the group consisting of: SEQ ID NOS: 1, 2, or 13-16. In some cases, the second guide RNA comprises a nucleotide sequence selected from the group consisting of: SEQ ID NOs: 3-12 or 17-26. In some cases, the CRISPR-associated endonuclease is a Class 1 or a Class 2 CRISPR-associated endonuclease. In some cases, the Class 1 CRISPR-associated endonuclease is selected from the group consisting of: Cas3, Cas5, Cas8a, Cas8b, Cas8c, Cas10d, Cse1, Cse2, Csy1, Csy2, Csy3, GSU0054, Cas10, Csm2, Cmr5, Csx11, Csx10, and Csf1. In some cases, the Class 2 CRISPR-associated endonuclease is selected from the group consisting of: Cas9, Cas12a, Csn2, Cas4, Cas12b, Cas12c, Cas13a, Cas13b, Cas13c, and Cas13d. In some cases, the CRISPR-associated endonuclease comprises an amino acid sequence having at least 80% sequence identity to a wild-type CRISPR-associated endonuclease. In some cases, the CRISPR-associated endonuclease is Cas9 or a variant thereof. In some cases, the Cas9 is a Streptococcus pyogenes Cas9 (spCas9). In some cases, the Cas9 variant comprises one or more point mutations, relative to a wild-type Streptococcus pyogenes Cas9 (spCas9), selected from the group consisting of: R780A, K810A, K848A, K855A, H982A, K1003A, R1060A, D1135E, N497A, R661A, Q695A, Q926A, L169A, Y450A, M495A, M694A, and M698A.

In yet another aspect, a kit for genotyping CYP2D6 is provided, comprising: (a) a Clustered Regularly Interspaced Short Palindromic Repeat (CRISPR)-associated endonuclease; (b) a first guide RNA (gRNA) comprising a nucleotide sequence substantially complementary to a nucleotide sequence present in genomic DNA that is upstream of a genetic locus comprising CYP2D6, CYP2D7, and CYP2D8; and (c) a second guide RNA (gRNA) comprising a nucleotide sequence substantially complementary to a nucleotide sequence present in genomic DNA that is downstream of the genetic locus comprising CYP2D6, CYP2D7, and CYP2D8. In some cases, the first guide RNA comprises a nucleotide sequence selected from the group consisting of: SEQ ID NOS: 1, 2, or 13-16. In some cases, the second guide RNA comprises a nucleotide sequence selected from the group consisting of: SEQ ID NOs: 3-12 or 17-26. In some cases, the CRISPR-associated endonuclease is a Class 1 or a Class 2 CRISPR-associated endonuclease. In some cases, the Class 1 CRISPR-associated endonuclease is selected from the group consisting of: Cas3, Cas5, Cas8a, Cas8b, Cas8c, Cas10d, Cse1, Cse2, Csy1, Csy2, Csy3, GSU0054, Cas10, Csm2, Cmr5, Csx11, Csx10, and Csf1. In some cases, the Class 2 CRISPR-associated endonuclease is selected from the group consisting of: Cas9, Cas12a, Csn2, Cas4, Cas12b, Cas12c, Cas13a, Cas13b, Cas13c, and Cas13d. In some cases, the CRISPR-associated endonuclease comprises an amino acid sequence having at least 80% sequence identity to a wild-type CRISPR-associated endonuclease. In some cases, the CRISPR-associated endonuclease is Cas9 or a variant thereof. In some cases, the Cas9 is a Streptococcus pyogenes Cas9 (spCas9). In some cases, the Cas9 variant comprises one or more point mutations, relative to a wild-type Streptococcus pyogenes Cas9 (spCas9), selected from the group consisting of: R780A, K810A, K848A, K855A, H982A, K1003A, R1060A, D1135E, N497A, R661A, Q695A, Q926A, L169A, Y450A, M495A, M694A, and M698A.

In yet another aspect, a system for analyzing a complex genomic region of interest is provided, the system comprising: (a) at least one memory location configured to receive a data input comprising data generated from a method comprising: (i) isolating high-molecular weight DNA from genomic DNA comprising the complex genomic region of interest; (ii) contacting the genomic DNA with a Clustered Regularly Interspaced Short Palindromic Repeat (CRISPR)-associated endonuclease and two or more gRNAs to excise the complex genomic region of interest, wherein the two or more gRNAs each comprise nucleotide sequences substantially complementary to different nucleotide sequences present in the genomic DNA, and wherein the different nucleotide sequences flank the complex genomic region of interest; and (iii) analyzing the complex genomic region of interest to generate the data, wherein the method does not involve DNA amplification; and (b) a computer processor operably coupled to the at least one memory location, wherein the computer processor is programmed to generate an output based on the data. In some cases, the output is a report. In some cases, the output is a genotype of the complex genomic region of interest. In some cases, the output is a genetic sequence of the complex genomic region of interest. In some cases, the output is a structural analysis of the complex genomic region of interest. In some cases, the analyzing comprises genotyping the complex genomic region of interest. In some cases, the analyzing comprises performing structural analysis of the complex genomic region of interest. In some cases, the analyzing comprises sequencing the complex genomic region of interest. In some cases, the sequencing comprises long-read sequencing. In some cases, the long-read sequencing comprises single-molecule real-time sequencing or nanopore sequencing. In some cases, the isolating of (i) is performed prior to the contacting of (ii). In some cases, the isolating of (i) is performed after the contacting of (ii). In some cases, the high-molecular weight DNA is at least 10 kilobases in length. In some cases, the complex genomic region of interest comprises a target gene and one or more pseudogenes thereof. In some cases, the one or more pseudogenes have at least 75% sequence identity to the target gene. In some cases, the complex genomic region of interest comprises CYP2D6, CYP2D7, and CYP2D8. In some cases, the complex genomic region of interest comprises CYP2C8, CYP2C9, CYP2C18, and CYP2C19. In some cases, the complex genomic region of interest comprises one or more repetitive regions, one or more duplications, one or more insertions, one or more inversions, one or more tandem repeats, one or more retrotransposons, or any combination thereof. In some cases, the complex genomic region of interest is a highly polymorphic gene locus. In some cases, the CRISPR-associated endonuclease is a Class 1 or a Class 2 CRISPR-associated endonuclease. In some cases, the Class 1 CRISPR-associated endonuclease is selected from the group consisting of: Cas3, Cas5, Cas8a, Cas8b, Cas8c, Cas10d, Cse1, Cse2, Csy1, Csy2, Csy3, GSU0054, Cas10, Csm2, Cmr5, Csx11, Csx10, and Csf1. In some cases, the Class 2 CRISPR-associated endonuclease is selected from the group consisting of: Cas9, Cas12a, Csn2, Cas4, Cas12b, Cas12c, Cas13a, Cas13b, Cas13c, and Cas13d. In some cases, the CRISPR-associated endonuclease comprises an amino acid sequence having at least 80% sequence identity to a wild-type CRISPR-associated endonuclease. In some cases, the CRISPR-associated endonuclease is Cas9 or a variant thereof. In some cases, the Cas9 is a Streptococcus pyogenes Cas9 (spCas9). In some cases, the Cas9 variant comprises one or more point mutations, relative to a wild-type Streptococcus pyogenes Cas9 (spCas9), selected from the group consisting of: R780A, K810A, K848A, K855A, H982A, K1003A, R1060A, D1135E, N497A, R661A, Q695A, Q926A, L169A, Y450A, M495A, M694A, and M698A. In some cases, the genomic DNA is not fragmented, digested, or sheared prior to a). In some cases, the genomic DNA is not subjected to restriction enzyme digestion prior to a). In some cases, the complex genomic region of interest is up to 250 kilobases in length. In some cases, the method further comprises, ligating one or more sequencing adapters to one or both ends of the excised genomic region of interest. In some cases, the method does not involve any one of polymerase chain reaction (PCR) or isothermal amplification. In some cases, the method does not involve any one of multiple displacement amplification (MDA), strand displacement amplification (SDA), nucleic acid sequence based amplification (NASBA), loop-mediated isothermal amplification, rolling circle amplification (RCA), ligase chain reaction (LCR), helicase dependent amplification, or ramification amplification method. In some cases, the genomic DNA is provided in a biological sample. In some cases, the biological sample comprises a body fluid (e.g., blood (e.g., whole blood, plasma, serum), urine, saliva, bone marrow, spinal fluid, sputum, ascites, lymphatic fluid, pleural fluid, amniotic fluid, semen, vaginal fluid, sweat, stool, glandular secretions, ocular fluids, breast milk) or a solid tissue sample. In some cases, the biological sample is a diagnostic sample.

In yet another aspect, a system for identifying genetic variation in CYP2D6 of a subject is provided, the system comprising: (a) at least one memory location configured to receive a data input comprising sequencing data generated from a method comprising: (ii) contacting genomic DNA obtained from the subject with a Clustered Regularly Interspaced Short Palindromic Repeat (CRISPR)-associated endonuclease and two or more gRNAs to excise a genetic locus comprising CYP2D6, CYP2D7, and CYP2D8; and (iii) performing long-read sequencing of the genetic locus to generate the sequencing data; and (b) a computer processor operably coupled to the at least one memory location, wherein the computer processor is programmed to generate an output based on the sequencing data. In some cases, the output is a report. In some cases, the output identifies genetic variation in CYP2D6. In some cases, the output identifies a decrease in, a loss of, or an increase in a function of CYP2D6. In some cases, the report recommends a treatment to the subject based on the genetic variation. In some cases, the report recommends a dosage of a therapeutic to the subject based on the genetic variation. In some cases, the report recommends altering a dosage of a therapeutic based on the genetic variation. In some cases, the therapeutic is a therapeutic that is activated by or metabolized by CYP2D6. In some cases, the method further comprises, prior to (ii), isolating high molecular weight DNA comprising the genetic locus. In some cases, the high molecular weight DNA is at least 40 kilobases in length. In some cases, the two or more gRNAs each comprise nucleotide sequences substantially complementary to different nucleotide sequences present in the genomic DNA, and wherein the different nucleotide sequences flank the genetic locus comprising CYP2D6, CYP2D7, and CYP2D8. In some cases, the two or more gRNAs comprise a nucleotide sequence selected from the group consisting of: SEQ ID NOS: 1-26. In some cases, the genetic locus is at least 40 kilobases in length. In some cases, the long-read sequencing comprises single-molecule real-time sequencing or nanopore sequencing. In some cases, the CRISPR-associated endonuclease is a Class 1 or a Class 2 CRISPR-associated endonuclease. In some cases, the Class 1 CRISPR-associated endonuclease is selected from the group consisting of: Cas3, Cas5, Cas8a, Cas8b, Cas8c, Cas10d, Cse1, Cse2, Csy1, Csy2, Csy3, GSU0054, Cas10, Csm2, Cmr5, Csx11, Csx10, and Csf1. In some cases, the Class 2 CRISPR-associated endonuclease is selected from the group consisting of: Cas9, Cas12a, Csn2, Cas4, Cas12b, Cas12c, Cas13a, Cas13b, Cas13c, and Cas13d. In some cases, the CRISPR-associated endonuclease comprises an amino acid sequence having at least 80% sequence identity to a wild-type CRISPR-associated endonuclease. In some cases, the CRISPR-associated endonuclease is Cas9 or a variant thereof. In some cases, the Cas9 is a Streptococcus pyogenes Cas9 (spCas9). In some cases, the Cas9 variant comprises one or more point mutations, relative to a wild-type Streptococcus pyogenes Cas9 (spCas9), selected from the group consisting of: R780A, K810A, K848A, K855A, H982A, K1003A, R1060A, D1135E, N497A, R661A, Q695A, Q926A, L169A, Y450A, M495A, M694A, and M698A. In some cases, the genomic DNA is not fragmented, digested, or sheared prior to (a). In some cases, the genomic DNA is not subjected to restriction enzyme digestion prior to (a). In some cases, the method further comprises, ligating one or more sequencing adapters to one or both ends of the excised genomic region of interest. In some cases, the method does not involve DNA amplification. In some cases, the method does not involve any one of polymerase chain reaction (PCR) or isothermal amplification. In some cases, the method does not involve any one of multiple displacement amplification (MDA), strand displacement amplification (SDA), nucleic acid sequence based amplification (NASBA), loop-mediated isothermal amplification, rolling circle amplification (RCA), ligase chain reaction (LCR), helicase dependent amplification, or ramification amplification method. In some cases, the biological sample is a body fluid (e.g., blood (e.g., whole blood, plasma, serum), urine, saliva, bone marrow, spinal fluid, sputum, ascites, lymphatic fluid, pleural fluid, amniotic fluid, semen, vaginal fluid, sweat, stool, glandular secretions, ocular fluids, breast milk) or a solid tissue sample.

In another aspect, a system for analyzing a genomic region of interest is provided, the system comprising: (a) at least one memory location configured to receive a data input comprising data generated from a method comprising: (i) contacting genomic DNA comprising the genomic region of interest with a Clustered Regularly Interspaced Short Palindromic Repeat (CRISPR)-associated endonuclease and an outer pair of guide RNAs (gRNAs), thereby generating a first excised fragment comprising the genomic region of interest; (ii) contacting the first excised fragment with a CRISPR-associated endonuclease and an inner pair of gRNAs, thereby generating a second excised fragment comprising the genomic region of interest; and (iii) analyzing the genomic region of interest contained within the second excised fragment; and (b) a computer processor operably coupled to the at least one memory location, wherein the computer processor is programmed to generate an output based on the data. In some cases, the output is a report. In some cases, the output is a genotype of the genomic region of interest. In some cases, the output is a genetic sequence of the genomic region of interest. In some cases, the output is a structural analysis of the genomic region of interest. In some cases, the analyzing comprises genotyping the genomic region of interest. In some cases, the analyzing comprises performing structural analysis of the genomic region of interest. In some cases, the analyzing comprises sequencing the genomic region of interest. In some cases, the sequencing comprises long-read sequencing. In some cases, the long-read sequencing comprises single-molecule real-time sequencing or nanopore sequencing. In some cases, the CRISPR-associated endonuclease and the outer pair of gRNAs of (i) associate with and block the 5′ and 3′ ends of the first excised fragment. In some cases, the method further comprises, prior to (ii), contacting the product of (i) with one or more exonucleases, such that background genomic DNA is digested and the first excised fragment is not digested. In some cases, the one or more exonucleases are selected from the group consisting of: exonuclease I, exonuclease II, exonuclease III, exonuclease IV, exonuclease V, exonuclease VI, exonuclease VII, exonuclease VIII, and any combination thereof. In some cases, the outer pair of gRNAs comprises a first outer gRNA and a second outer gRNA. In some cases, the first outer gRNA comprises a nucleotide sequence that is substantially complementary to a first nucleotide sequence present in the genomic DNA, and the second outer gRNA comprises a nucleotide sequence that is substantially complementary to a second nucleotide sequence present in the genomic DNA. In some cases, the first nucleotide sequence and the second nucleotide sequence are different. In some cases, the first nucleotide sequence and the second nucleotide sequence flank the genomic region of interest. In some cases, the first nucleotide sequence, the second nucleotide sequence, or both, are present in the genomic DNA up to about 100 kilobases in length from the genomic region of interest. In some cases, the inner pair of gRNAs comprises a first inner gRNA and a second inner gRNA. In some cases, the first inner gRNA comprises a nucleotide sequence that is substantially complementary to a third nucleotide sequence present in the genomic DNA, and the second inner gRNA comprises a nucleotide sequence that is substantially complementary to a fourth nucleotide sequence present in the genomic DNA. In some cases, the third nucleotide sequence and the fourth nucleotide sequence are different. In some cases, the third nucleotide sequence and the fourth nucleotide sequence flank the genomic region of interest. In some cases, the third nucleotide sequence and the fourth nucleotide sequence are present on the genomic DNA at a base length closer to the genomic region of interest than the first nucleotide sequence and the second nucleotide sequence. In some cases, the second excised fragment is smaller in base length than the first excised fragment. In some cases, the analyzing comprises sequencing the genomic region of interest contained within the second excised fragment. In some cases, the genomic DNA is provided at an amount of about 10 μg or greater. In some cases, the analyzing comprises genotyping the genomic region of interest contained within the second excised fragment. In some cases, the analyzing comprises performing structural analysis on the genomic region of interest contained within the second excised fragment. In some cases, the method further comprises, prior to (ii), isolating the first excised fragment. In some cases, the method further comprises, prior to (iii), isolating the second excised fragment. In some cases, the method does not involve DNA amplification. In some cases, the method further comprises, prior to (iii), attaching one or more adapters to the 5′ end, the 3′ end, or both, of the second excised fragment. In some cases, the CRISPR-associated endonuclease is a Class 1 CRISPR-associated endonuclease or a Class 2 CRISPR-associated endonuclease. In some cases, the Class 1 CRISPR-associated endonuclease is selected from the group consisting of: Cas3, Cas5, Cas8a, Cas8b, Cas8c, Cas10d, Cse1, Cse2, Csy1, Csy2, Csy3, GSU0054, Cas10, Csm2, Cmr5, Csx11, Csx10, and Csf1. In some cases, the Class 2 CRISPR-associated endonuclease is selected from the group consisting of: Cas9, Cas12a, Csn2, Cas4, Cas12b, Cas12c, Cas13a, Cas13b, Cas13c, and Cas13d. In some cases, the CRISPR-associated endonuclease comprises an amino acid sequence having at least 80% sequence identity to a wild-type CRISPR-associated endonuclease. In some cases, the CRISPR-associated endonuclease is Cas9 or a variant thereof. In some cases, the Cas9 is a Streptococcus pyogenes Cas9 (spCas9). In some cases, the Cas9 variant comprises one or more point mutations, relative to a wild-type Streptococcus pyogenes Cas9 (spCas9), selected from the group consisting of: R780A, K810A, K848A, K855A, H982A, K1003A, R1060A, D1135E, N497A, R661A, Q695A, Q926A, L169A, Y450A, M495A, M694A, and M698A. In some cases, the genomic DNA is not fragmented, digested, or sheared prior to (i). In some cases, the genomic DNA is not subjected to restriction enzyme digestion prior to (i). In some cases, the genomic region of interest is a complex genomic region. In some cases, the complex genomic region comprises a gene of interest and one or more pseudogenes thereof. In some cases, the one or more pseudogenes comprise a nucleotide sequence having at least 75% sequence identity to the gene of interest. In some cases, the complex genomic region comprises one or more repetitive regions, one or more duplications, one or more insertions, one or more inversions, one or more tandem repeats, one or more retrotransposons, or any combination thereof. In some cases, the genomic region of interest is a highly polymorphic gene locus. In some cases, the first excised fragment is at least about 0.06 kilobases in length. In some cases, the first excised fragment is up to about 200 kilobases in length. In some cases, the second excised fragment is at least about 0.02 kilobases in length. In some cases, the second excised fragment is up to about 199.98 kilobases in length. In some cases, the method does not involve any one of polymerase chain reaction (PCR) or isothermal amplification. In some cases, the method does not involve any one of multiple displacement amplification (MDA), strand displacement amplification (SDA), nucleic acid sequence based amplification (NASBA), loop-mediated isothermal amplification, rolling circle amplification (RCA), ligase chain reaction (LCR), helicase dependent amplification, or ramification amplification method. In some cases, the genomic DNA is provided or obtained in a biological sample. In some cases, the biological sample comprises a body fluid (e.g., blood (e.g., whole blood, plasma, serum), urine, saliva, bone marrow, spinal fluid, sputum, ascites, lymphatic fluid, pleural fluid, amniotic fluid, semen, vaginal fluid, sweat, stool, glandular secretions, ocular fluids, breast milk) or a solid tissue sample. In some cases, the biological sample is a diagnostic sample. In some cases, the genomic region of interest is a genetic locus comprising CYP2D6, CYP2D7, and CYP2D8. In some cases, the analyzing comprises identifying one or more genetic variations in CYP2D6. In some cases, the output comprises an identification of a subject as having a reduction, a loss of, or an increase in CYP2D6 function based on the genetic variation. In some cases, the output comprises a recommendation of a treatment or an alternative treatment to the subject based on the identification. In some cases, when the subject is identified as having a reduction in, a loss of, or an increase in CYP2D6 function, the output further comprises a recommendation of an alternative treatment to the subject. In some cases, the output further provides a recommendation of a dosage of a therapeutic to the subject based on the identification. In some cases, when the subject is identified as having a reduction in, a loss of, or an increase in CYP2D6 function, the output further comprises a recommendation to alter a dosage of a therapeutic. In some cases, the outer pair of gRNAs, the inner pair of gRNAs, or both, comprise gRNAs selected from any one of SEQ ID NOS: 1-418.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 depicts the CYP2D6 locus, according to embodiments provided herein. Panel A depicts the orientation of the reference gene locus containing a single copy of the CYP2D6 gene in relation to CYP2D7 and CYP2D8. Representative examples of structural variants illustrating the complexity of CYP2D6 gene copy number variation, including complete CYP2D6 deletion (Panel B), duplication (Panel C), and presence of either a 5′ (Panel D) or 3′ (Panel E) CYPD6/CYPD7 hybrid allele. The duplicated gene in such arrangements often has a CYP2D7-like downstream region including the 1.6 kb long spacer sequence. The 5′-3′ orientation is shown relative to the reference sequence (NG_008376.3).

FIG. 2 depicts a non-limiting example of a flowchart depicting a method of isolating and sequencing the CYP2D6 locus, according to embodiments provided herein.

FIG. 3 depicts a non-limiting example of a comparison of genomic DNA extraction, according to embodiments provided herein. Lane A is 50 ng of gDNA extracted from lymphoblastoid cell line (LCL) cells with a modified high molecular weight protocol (>50 kb), lane B is 50 ng of gDNA extracted with Maxwell Rapid Sample Concentrator (RSC) (˜10-48 kb), lane C is 50 ng of gDNA control (Coriell; ˜10 kb-50 kb), lane D is lambda phage DNA (˜50 kDa; NEB), and lane E is HINDIII lambda phage digest.

FIG. 4A and FIG. 4B depict a non-limiting example of the design and validation of sgRNAs targeting the CYP2D6 locus, according to embodiments provided herein. FIG. 4A depicts a schematic of the necessary CRISPR cut sites to capture allele CYP2D6 and hybrid alleles. FIG. 4B depicts CRISPR Cut XL-PCR amplicons of target site. Sample A received Cas9 with no sgRNA, Sample B received Cas9 with sgRNA_1, and Sample C received Cas9 with sgRNA_2.

FIG. 5A and FIG. 5B depict a non-limiting example of efficiency of sgRNAs targeting the CYP2D6 locus on genomic DNA, according to embodiments of the disclosure. FIG. 5A depicts a gel image of XL-PCR products containing the sgRNA binding sites for regions up- and downstream of CYP2D6. Lane C is control. FIG. 5B depicts percentage of uncut gDNA normalized to the negative control. *=P-value <0.010.

FIG. 6 depicts a non-limiting example of NGS alignment of XL-PCR and NGS-based analysis approaches, according to embodiments of the disclosure.

FIGS. 7A-7C depict a non-limiting examples of issues with alternative CRISPR/Cas9 design approaches for the CYP2D6 locus, according to embodiments of the disclosure. Cutting sites are indicated with scissors. Xs represent alleles in which the shown design on the A allele would generate unwanted cutting on the B-E allele arrangements.

FIG. 8 depicts a non-limiting example of a comprehensive target design for the CYP2D6 locus. Cutting sites are indicated with scissors. Check marks represent alleles in which the shown design on the A allele would generate only on-target cutting on the B-E allele arrangements.

FIGS. 9A-9C depicts a non-limiting example of design and validation of sgRNAs targeting the CYP2D6 locus. FIG. 9A depicts a schematic of the necessary cut sites to target to capture allele CYP2D6 and hybrid alleles. FIG. 9B and FIG. 9C depict CRISPR Cut XL-PCR amplicons of target site. Sample A received Cas9 with no sgRNA, Sample B received Cas9 with sgRNA_1, and Sample C received Cas9 with sgRNA_2.

FIG. 10 depicts a non-limiting example of isolated of high molecular weight DNA according to embodiments of the disclosure. 2% DNA agarose gel of 100 ng high molecular weight genomic DNA extracted from LCL-cell pellets compared to lambda control and pre-extracted DNA from the Coriell Institute.

FIG. 11A and FIG. 11B depict a non-limiting example of sequence run coverage, according to embodiments disclosed herein.

FIG. 12A and FIG. 12B depict a non-limiting example sequence alignment size, according to embodiments disclosed herein.

FIG. 13 depicts a non-limiting example of an alignment plot, according to embodiments disclosed herein. 121× coverage of the targeted capture region was achieved. Boxes outline CYP2D6 and CYP2D7.

FIG. 14 depicts a non-limiting example of a Sashimi plot showing sgRNA specificity, according to embodiments disclosed herein. This plot shows the aligned region for the two sequencing runs. The upper alignment shows sequence data from the run using the sgRNAs designed to capture the region-of-interest (ROI) (chr22:42, 122, 115-41, 161, 320). The lower alignment shows enrichment performed on the same DNA sample using sgRNAs targeting the opposite strands.

FIG. 15 depicts a non-limiting example of a Sashimi plot showing sgRNA specificity for multiple complex structural arrangements, according to embodiments disclosed herein. This plot shows the aligned region for four sequencing runs. The sequence data from the runs uses the sgRNAs designed to capture the region-of-interest (ROI) (chr22:42, 122, 115-41, 161, 320) and includes four different structural events: (1) Deletion of CYP2D6 on one allele; (2) Hybrid allele in tandem with CYP2D6 on one allele; (3) Duplication event on one allele; and (4) Deletion of CYP2D6 on one allele and duplication of CYP2D6 on the second allele.

FIG. 16 depicts a non-limiting example of a computer system in accordance with embodiments provided herein.

FIG. 17 depicts a non-limiting example of a nested enrichment approach for analyzing complex genomic regions of interest, in accordance with embodiments provided herein.

FIG. 18 depicts non-limiting representative fold change data for the ROI when using the nested enrichment approach for analyzing complex genomic regions of interest. As shown in the figure, different pairs of outer gRNAs used to perform the nested enrichment prior to DNA digest and subsequent CRISPR reaction with second inner gRNAs generates significant enrichment of the ROI for downstream applications compared to samples that received only the inner gRNAs.

### DETAILED DESCRIPTION

Disclosed herein are methods for analyzing a genomic region of interest (ROI) (e.g., from genomic DNA). The region of interest can be, e.g., a complex (e.g., a highly-complex) genomic region. The complex genomic region may include, e.g., a highly polymorphic region, a region comprising a target gene and one or more pseudogenes having high sequence homology to the target gene, a region comprising one or more repetitive elements, one or more inversions, one or more insertions, one or more duplications, one or more tandem repeats, one or more retrotransposons, and the like. The methods provided herein generally involve the use of a Clustered Interspaced Short Palindromic Repeat (CRISPR)-associated endonuclease and two or more guide RNAs (gRNAs) to excise the region of interest from genomic DNA.

In one aspect, the disclosure provides a nested enrichment approach for enriching and analyzing a complex genomic region of interest. The nested enrichment approach generally involves the use of a CRISPR-associated endonuclease in combination with an outer pair of gRNAs (e.g., a first outer gRNA and a second outer gRNA) and/or an inner pair of gRNAs (e.g., a first inner gRNA and a second inner gRNA). The method involves excising a fragment from genomic DNA containing the genomic region of interest using a CRISPR-associated endonuclease and the outer pair of gRNAs to generate a first excised fragment comprising the genomic region of interest. The methods further comprise excising from the first excised fragment a smaller fragment to generate a second excised fragment comprising the genomic region of interest by using a CRISPR-associated endonuclease and the inner pair of gRNAs. In some cases, the method further involves digesting background DNA with one or more exonucleases.

The methods provided herein further involve analyzing the genomic region of interest (e.g., located on the second fragment) (e.g., by sequencing, e.g., via long-read sequencing methods, by genotyping, by performing structural analysis). Further provided herein are methods of analyzing the CYP2D6 locus (e.g., comprising the target gene CYP2D6, and the pseudogenes CYP2D7 and CYP2D8). Advantageously, in some embodiments, the methods do not involve the use of DNA amplification (e.g., amplification-free). The methods may improve the accuracy of sequencing complex (e.g., highly complex) genomic regions (e.g., reduce the sequencing error rate) (e.g., as compared to traditional methods), and/or may reduce the time for sequencing complex (e.g., highly-complex) genomic regions (e.g., as compared to traditional methods), and/or may decrease the cost of sequencing complex genomic (e.g., highly-complex) regions (e.g., as compared to traditional methods). Additionally, the methods provided herein may allow for the use of higher starting material (e.g., higher amounts of genomic DNA) than standard CRISPR-based approaches. Additionally provided herein are systems for performing the methods provided herein, as well as compositions and kits comprising a CRISPR-associated endonuclease and two or more gRNAs that excise a genomic region of interest (e.g., the CYP2D6 locus (e.g., to excise the CYP2D6 locus from genomic DNA)).

As used herein and in the appended claims, the singular forms “a,” “an,” and, “the” include plural referents unless the context clearly dictates otherwise. It is further noted that the claims can be drafted to exclude any optional element. As such, this statement is intended to serve as antecedent basis for use of such exclusive terminology as “solely,” “only,” and the like in connection with the recitation of claim elements, or use of a “negative” limitation.

Certain ranges or numbers are presented herein with numerical values being preceded by the term “about”. The term “about” is used herein to mean plus or minus 1%, 2%, 3%, 4%, or 5% of the number that the term refers to. As used herein, the terms “subject” and “individual”, are used interchangeably and can be any animal, including mammals (e.g., a human or non-human animal).

As used herein, the term “CYP2D6” can refer to the CYP2D6 gene or any structural variant or single gene copy variant thereof. Structural variants of CYP2D6 can include gene-fusions, hybrids with neighboring highly homologous pseudogenes (e.g., CYP2D7 and CYP2D8), copy number variations (CNVs), gene duplications and multiplications, tandem repeats, and rearrangements. One example of CYP2D6 structural variants is the presence of CYP2D7 derived sequence in exon 9 of CYP2D6 (referred to as “exon 9 conversion”). Single gene copy variants can include single nucleotide polymorphisms (SNPs) or insertions or deletions of nucleotides (indels). An allele of CYP2D6 can be a structural variant or single gene copy variant, including, but not limited to, any one of: *1, *1×N, *2, *2×N, *2A, *2A×N, *35, *35×N, *9, *9×N, *10, *10×N, *17, *17×N, *29, *29×N, *36-*10, *36-*10×N, *36×N-*10, *36×N-*10×N, *41, *41×N, *3, *3×N, *4, *4×N, *4N, *5, *6, *6×N, *36, and *36×N. In some cases, each allele of the CYP2D6 is a different structural variant or single gene copy variant. In some cases, each allele of the CYP2D6 is identical.

The term “CYP2D6 locus” as used herein refers to a genomic region comprising the CYP2D6 gene, and the highly-homologous pseudogenes CYP2D7 and CYP2D8. In humans, the CYP2D6 locus is found on chromosome 22. In some embodiments, the methods provided herein involve analyzing (e.g., sequencing, genotyping, performing structural analysis) part of or the entire CYP2D6 locus (e.g., including the CYP2D6 gene, and the highly homologous pseudogenes CYP2D7 and CYP2D8). In some embodiments, the methods provided herein involve excising part of or the entire CYP2D6 locus (e.g., including the CYP2D6 gene, and the highly homologous pseudogenes CYP2D7 and CYP2D8) from genomic DNA (e.g., by using a CRISPR-associated endonuclease and two or more gRNAs that target genomic sequences flanking the CYP2D6 locus).

As used herein, the term “CRISPR/Cas nuclease system” refers to a complex comprising a guide RNA (gRNA) and a CRISPR-associated endonuclease (Cas protein). The term “CRISPR” can refer to the Clustered Regularly Interspaced Short Palindromic Repeats and the related system thereof. The CRISPR/Cas nuclease system can be a Class 1 or a Class 2 CRISPR/Cas nuclease system. The CRISPR/Cas nuclease system can be a type I, type II, type III, type IV, type V, or type VI CRISPR/Cas nuclease system. The gRNA can interact with the Cas protein to direct the nuclease activity of the Cas protein to a target sequence. The target sequence can comprise a “protospacer” and a “protospacer adjacent motif” (PAM), and both domains may be needed for a Cas mediated activity (e.g., cleavage). The gRNA can pair with (or hybridize to) a binding site on the opposite strand of the protospacer to direct the Cas to the target sequence. The PAM site can refer to a short sequence recognized by the Cas protein and, in some cases, can be required for the Cas protein activity.

As used herein, the terms “Cas” or “Cas protein” refer to a protein of or derived from a CRISPR/Cas system having endonuclease activity. In some cases, a CRISPR-associated endonuclease, as used herein, as a Cas protein. A Cas protein can be a naturally occurring Cas protein, a non-naturally occurring Cas protein, or a fragment thereof. In some cases, a Cas protein is a variant of a naturally-occurring Cas protein (e.g., having one or more amino acid substitutions, insertions, deletions, etc. relative to a naturally-occurring Cas protein). In some cases, the Cas protein is a Class I Cas protein, non-limiting examples including, Cas3, Cas8a, Cas5, Cas8b, Cas8c, Cas10d, Cse1, Cse2, Csy1, Csy2, Csy3, GSU0054, Cas10, Csm2, Cmr5, Cas10, Csx11, Csx10, and Csf1. In some cases, the Cas protein is a Class II Cas protein, non-limiting examples including, Cas9, Csn2, Cas4, Cas12a (Cpf1), Cas12b (C2cl), Cas12c (C2c3), Cas13a (C2c2), Cas13b, Cas13c, and Cas13d. In some cases, the Cas protein is Cas9. In some cases, the Cas protein is Cas12a.

The terms “guide RNA” or “gRNA” are used interchangeably herein and generally refer to an RNA molecule (or a group of RNA molecules, collectively) that can bind to a Cas protein and aid in targeting the Cas protein to a specific location within a target polynucleotide (e.g., a DNA). A guide RNA can comprise a CRISPR RNA (crRNA) segment, and, optionally, a trans-activating crRNA (tracrRNA) segment. The term “crRNA”, as used herein, can refer to an RNA molecule or portion thereof that includes a polynucleotide-targeting guide sequence, a stem sequence, and, optionally, a 5′-overhang sequence. The crRNA can bind to a binding site. The term “tracrRNA”, as used herein, can refer to an RNA molecule or portion thereof that includes a protein-binding segment (e.g., the protein-binding segment is capable of interacting with a CRISPR-associated protein, e.g., Cas9). The term “guide RNA” can refer to a single guide RNA (sgRNA), where the crRNA segment and the optional tracrRNA segment are located in the same RNA molecule. The term “guide RNA” can also refer to, collectively, a group of two or more RNA molecules, where the crRNA and the tracrRNA are located in separate RNA molecules.

The term “long-read sequencing” (also termed “third generation sequencing”) as used herein generally refers to any sequencing method that is capable of generating substantially longer sequencing reads (>10,000 bp) than second generation sequencing. In some embodiments, the methods provided herein involve the use of long-read sequencing (e.g., to genotype complex genomic regions of interest). Non-limiting examples of long-read sequencing systems include those developed by Pacific Biosciences, Oxford Nanopore Technology, Quantapore, Stratos, and Helicos. In some cases, the long-read sequencing method is single molecule real time sequencing (SMRT) (e.g., developed by Pacific Biosciences). In some cases, the long-read sequencing method is nanopore sequencing (e.g., MinION, GridION, and PromethION, developed by Oxford Nanopore Technology). In some cases, long-read sequencing encompasses any long-read sequencing method or system (e.g., third generation sequencing method or system) currently under development or to be developed in the future.

The term “nucleic acid amplification” as used herein generally refers to any method of generating multiple copies of a target nucleic acid (e.g., DNA) from a single nucleic acid molecule. The target nucleic acid can be DNA (e.g., DNA amplification) or RNA (e.g., RNA amplification). Nucleic acid amplification includes polymerase chain reaction (PCR) and any and all variants or modifications thereof, as well as alternative types of nucleic acid amplification methods, such as, but not limited to, loop mediated isothermal amplification (LAMP), nucleic acid sequence based amplification (NASBA), strand displacement amplification (SDA), multiple displacement amplification (MDA), rolling circle amplification (RCA), ligase chain reaction (LCR), helicase dependent amplification, and ramification amplification method (RAM). In various aspects of the disclosure, the methods provided herein do not involve the use of nucleic acid (e.g., DNA) amplification (e.g., amplification-free).

### Methods of the Disclosure

The disclosure herein generally provides a nested enrichment approach for enriching for and analyzing (e.g., sequencing, genotyping, structural analysis) a genomic region of interest (e.g., a complex genomic region of interest). In various aspects, the method comprises contacting genomic DNA comprising the genomic region of interest (e.g., complex genomic region of interest) with a Clustered Regularly Interspaced Short Palindromic Repeat (CRISPR)-associated endonuclease and an outer pair of guide RNAs (gRNAs), thereby generating a first excised fragment comprising the genomic region of interest. In various aspects, the method further comprises contacting the first excised fragment with a CRISPR-associated endonuclease and an inner pair of gRNAs, thereby generating a second (e.g., smaller) excised fragment comprising the genomic region of interest. In various aspects, the method further comprises analyzing (e.g., sequencing, genotyping, structural analysis) the genomic region of interest (e.g., present in the second excised fragment).

In various aspects, the method involves contacting genomic DNA comprising the genomic region of interest (e.g., complex genomic region of interest) with a Clustered Regularly Interspaced Short Palindromic Repeat (CRISPR)-associated endonuclease and an outer pair of guide RNAs (gRNAs). The outer pair of gRNAs may comprise a first outer gRNA and a second outer gRNA.

The first and second outer gRNAs comprise a nucleotide sequence that is substantially complementary to nucleotide sequences present in the genomic DNA. Generally, the first and second outer gRNAs are substantially complementary to different nucleotide sequences present in the genomic DNA. The first and second outer gRNA sequences are selected such that they are substantially complementary to nucleotide sequences that flank the genomic region of interest. For example, the first outer gRNA may be substantially complementary to a nucleotide sequence that is upstream of the genomic region of interest, and the second outer gRNA may be substantially complementary to a nucleotide sequence that is downstream of the genomic region of interest, or vice versa. Generally, contacting the genomic DNA with the CRISPR-associated endonuclease and the outer pair of gRNAs results in excision of a fragment of the genomic DNA (e.g., a first excised fragment) containing the genomic region of interest (e.g., complex genomic region of interest).

The first and second outer gRNAs may be substantially complementary to nucleotide sequences (e.g., present in the genomic DNA) that are at a base length of up to about 30 kilobases from (e.g., upstream and/or downstream) the genomic region of interest. For example, the first and second outer gRNAs may be substantially complementary to nucleotide sequences (e.g., present in the genomic DNA) that are at a base length of at least about 5 kilobases, at least about 10 kilobases, at least about 15 kilobases, at least about 20 kilobases, at least about 25 kilobases, or more, from (e.g., upstream and/or downstream) the genomic region of interest.

Without wishing to be bound by theory, it is thought that, after excision of the first fragment, the CRISPR-associated endonuclease and the outer pair of gRNAs remain associated with and block the 5′ and 3′ ends of the first excised fragment. Advantageously, this feature may be used to remove background genomic DNA. In one preferred embodiment, the first excised fragment (and remaining genomic DNA) are contacted with one or more exonucleases. The one or more exonucleases are capable of digesting background DNA while leaving the blocked fragment intact. The one or more exonucleases may be selected from the group consisting of: exonuclease I, exonuclease II, exonuclease III, exonuclease IV, exonuclease V, exonuclease VI, exonuclease VII, exonuclease VIII, and any combination thereof.

In various aspects, the method further comprises contacting the first excised fragment (e.g., containing the genomic region of interest) with a CRISPR-associated endonuclease and an inner pair of gRNAs. In some cases, the contacting occurs after the first excised fragment (and remaining genomic DNA) have been contacted with the one or more exonucleases, as described herein. The inner pair of gRNAs may comprise a first inner gRNA and a second inner gRNA.

The first and second inner gRNAs comprise nucleotide sequences that are substantially complementary to nucleotide sequences present in the first excised fragment (e.g., generated by contacting genomic DNA with a CRISPR-associated endonuclease and the outer pair of gRNAs, as described herein). Generally, the first and second inner gRNAs are substantially complementary to different nucleotide sequences present in the first excised fragment (e.g., generated by contacting genomic DNA with a CRISPR-associated endonuclease and the outer pair of gRNAs, as described herein). The first and second inner gRNA sequences are selected such that they are substantially complementary to nucleotide sequences that flank the genomic region of interest. For example, the first inner gRNA may be substantially complementary to a nucleotide sequence that is upstream of the genomic region of interest, and the second inner gRNA may be substantially complementary to a nucleotide sequence that is downstream of the genomic region of interest, or vice versa. Generally, contacting the first excised fragment containing the genomic region of interest (e.g., generated by contacting genomic DNA with a CRISPR-associated endonuclease and the outer pair of gRNAs, as described herein) with the CRISPR-associated endonuclease and the inner pair of gRNAs results in excision of a second fragment (e.g., second excised fragment) containing the genomic region of interest.

The first and second inner gRNAs may be substantially complementary to nucleotide sequences (e.g., present in the first excised fragment) that are at a base length from about 0.06 to about 200 kilobases from (e.g., upstream and/or downstream) the genomic region of interest. Generally, the inner pair of gRNAs are nested such that they are substantially complementary to nucleotide sequences that are closer in base length to the genomic region of interest than the outer pair of gRNAs. Put another way, the inner pair of gRNAs, when used in conjunction with the CRISPR-associated endonuclease, as described herein, excise a smaller fragment (e.g., a second excised fragment) from the first excised fragment. Preferably, the second excised fragment comprises the (e.g., entire) genomic region of interest.

In various aspects, the method involves isolating genomic DNA comprising the genomic region of interest. In some embodiments, the method involves isolating high-molecular weight genomic DNA. In some embodiments, the method involves enriching for high molecular weight genomic DNA. In some embodiments, the high molecular weight genomic DNA is at least about 10 kilobases in length. For example, the high molecular weight genomic DNA is at least about 10 kilobases in length, at least about 15 kilobases in length, at least about 20 kilobases in length, at least about 30 kilobases in length, at least about 35 kilobases in length, at least about 40 kilobases in length, at least about 45 kilobases in length, at least about 50 kilobases in length, at least about 55 kilobases in length, at least about 60 kilobases in length, at least about 65 kilobases in length, at least about 70 kilobases in length, at least about 75 kilobases in length, at least about 80 kilobases in length, at least about 85 kilobases in length, at least about 90 kilobases in length, at least about 95 kilobases in length, or greater. In some embodiments, isolating high molecular weight genomic DNA ensures that the entire, intact genomic region of interest is contained in the sample. In some embodiments, isolation and/or enriching of high molecular weight genomic DNA is performed prior to the first CRISPR reaction (e.g., before the genomic DNA is contacted with the CRISPR-associated endonuclease and the outer pair of gRNAs). In some embodiments, isolation and/or enriching of high molecular weight genomic DNA is performed after performing the first CRISPR reaction (e.g., after the genomic DNA is contacted with the CRISPR-associated endonuclease and the outer pair of gRNAs).

In various aspects, the method involves any method for isolating high molecular weight genomic DNA. Non-limiting examples of methods for isolating high molecular weight genomic DNA include the NucleoBond® Genomic DNA and RNA purification system (as manufactured by Takara Bio), and the Nanobind CBB Big DNA kit (as manufactured by Circulomics).

In some aspects, isolating genomic DNA comprising the genomic region of interest can be performed prior to contacting the genomic DNA with the CRISPR-associated endonucleases and guide RNAs. In other aspects, isolating genomic DNA comprising the genomic region of interest can be performed after contacting the genomic DNA with the CRISPR-associated endonucleases and guide RNAs (e.g., after excising the genomic region of interest from the genomic DNA).

In various aspects, the starting amount of genomic DNA used in the method is at greater than what is commonly used in CRISPR-based approaches. In some cases, the starting amount of genomic DNA used in any method provided herein is at least about 1 μg (e.g., at least about 5 μg, at least about 10 μg, at least about 20 μg, at least about 50 μg, at least about 100 μg, at least about 500 μg, or more).

In various aspects, the genomic region of interest is a complex genomic region or a highly-complex genomic region. In some cases, the genomic region of interest is a highly polymorphic genomic region. In some cases, the genomic region of interest contains multiple repetitive elements or regions. In some cases, the genomic region of interest contains one or more target gene and one or more additional genes having high sequence identity to the target gene (e.g., having at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or greater sequence identity to the target gene). In some cases, the genomic region of interest contains one or more target gene and one or more pseudogenes having high sequence identity to the target gene (e.g., having at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or greater sequence identity to the target gene). In some cases, the genomic region of interest comprises one or more repetitive regions, one or more duplications, one or more insertions, one or more inversions, one or more tandem repeats, one or more retrotransposons, or any combination thereof. In some cases, the genomic region of interest is a genomic region that is generally difficult or challenging to analyze accurately by traditional methods (e.g., by short-read sequencing methods).

In some cases, the genomic region of interest is at least about 10 kilobases in length. For example, the genomic region of interest may be at least about 10 kilobases in length, at least about 15 kilobases in length, at least about 20 kilobases in length, at least about 25 kilobases in length, at least about 30 kilobases in length, at least about 35 kilobases in length, at least about 40 kilobases in length, at least about 45 kilobases in length, at least about 50 kilobases in length, at least about 55 kilobases in length, at least about 60 kilobases in length, at least about 65 kilobases in length, at least about 70 kilobases in length, at least about 75 kilobases in length, at least about 80 kilobases in length, at least about 85 kilobases in length, at least about 90 kilobases in length, at least about 95 kilobases in length, at least about 100 kilobases in length, at least about 110 kilobases in length, at least about 120 kilobases in length, at least about 130 kilobases in length, at least about 140 kilobases in length, at least about 150 kilobases in length, at least about 160 kilobases in length, at least about 170 kilobases in length, at least about 180 kilobases in length, at least about 190 kilobases in length, at least about 200 kilobases in length, at least about 210 kilobases in length, at least about 220 kilobases in length, at least about 230 kilobases in length, at least about 240 kilobases in length, or at least about 250 kilobases in length. In some aspects, the genomic region of interest is greater than about 10 kilobases in length. In some aspects, the genomic region of interest is less than about 250 kilobases in length.

The CRISPR-associated endonuclease can be any CRISPR-associated endonuclease described herein. In some cases, the CRISPR-associated endonuclease is a Class I or a Class II CRISPR-associated endonuclease. Non-limiting examples of Cas I CRISPR-associated endonucleases include, Cas3, Cas5, Cas8a, Cas8b, Cas8c, Cas10d, Cse1, Cse2, Csy1, Csy2, Csy3, GSU0054, Cas10, Csm2, Cmr5, Csx11, Csx10, and Csf1. Non-limiting examples of Class II CRISPR-associated endonucleases include, Cas9, Cas12a, Csn2, Cas4, Cas12b, Cas12c, Cas13a, Cas13b, Cas13c, and Cas13d. In some cases, the CRISPR-associated endonuclease is a Cas protein or polypeptide. In some embodiments, the CRISPR-associated endonuclease is a Cas12a protein or polypeptide.

In some embodiments, the CRISPR-associated endonuclease is a Cas9 protein or polypeptide. In some cases, the Cas9 protein or polypeptide is derived from the bacterial species Streptococcus pyogenes. In some cases, the Cas9 protein or polypeptide has an amino acid sequence identical to a wild-type Cas9 amino acid sequence. In other cases, the Cas9 protein or polypeptide has an amino acid sequence that is modified relative to a wild-type Cas9 amino acid sequence. In some cases, the Cas9 protein or polypeptide has one or more mutations (e.g., relative to a wild-type Cas9 protein or polypeptide). In some cases, the one or more mutations is a substitution, a deletion, or an insertion. The Cas9 protein or polypeptide may have an amino acid sequence having at least about 50% sequence identity relative to a wild-type Cas9 protein or polypeptide. For example, the Cas9 protein or polypeptide may have at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity relative to a wild-type Cas9 protein or polypeptide. In some cases, the Cas9 variant may comprise one or more point mutations relative to a wild-type S. pyogenes Cas9. For example, the Cas9 variant may comprise a point mutation relative to a wild-type S. pyogenes Cas9 selected from the group consisting of: R780A, K810A, K848A, K855A, H982A, K1003A, R1060A, D1135E, N497A, R661A, Q695A, Q926A, L169A, Y450A, M495A, M694A, and M698A.

In various aspects, the method involves the use of gRNAs (e.g., an outer pair of gRNAs and/or an inner pair of gRNAs). The gRNAs may be CRISPR RNA (crRNA) or single guide RNA (sgRNA). In some embodiments, the gRNAs comprise nucleotide sequences that are complementary or substantially complementary to target nucleotide sequences, such that the gRNAs are capable of binding to the target nucleotide sequences, and directing the CRISPR complex to the desired cut site. In some embodiments, each of the gRNAs (e.g., inner gRNAs, outer gRNAs) bind to different target nucleotide sequences. In some embodiments, at least one of the gRNAs is complementary or substantially complementary to a region upstream of the genomic region of interest, and at least one of gRNAs is complementary or substantially complementary to a region downstream of the genomic region of interest. For example, at least one of the outer gRNAs is complementary or substantially complementary to a region upstream of the genomic region of interest, and at least one of the outer gRNAs is complementary or substantially complementary to a region downstream of the genomic region of interest. Similarly, at least one of the inner gRNAs is complementary or substantially complementary to a region upstream of the genomic region of interest, and at least one of the inner gRNAs is complementary or substantially complementary to a region downstream of the genomic region of interest. In some embodiments, the gRNA pairs (e.g., inner pair of gRNAs, outer pair of gRNAs) bind to target sequences that flank the genomic region of interest. Generally, the gRNAs are designed such that they each target a genomic sequence that is outside of the genomic region of interest, such that the contacting (e.g., with the CRISPR-associated endonuclease and the pair of outer or inner gRNAs) excises the entire genomic region of interest.

In various aspects, the methods further involve analyzing the genomic region of interest. In some cases, the analyzing comprises genotyping the genomic region of interest. Genotyping may include a process of identifying differences in the genetic make-up of the genomic region of interest by using one or more assays to examine the sequence of the genomic region of interest and, in some cases, comparing the sequence to another sequence (e.g., a reference sequence). Genotyping may be performed by any known method, including, but not limited to, DNA sequencing, restriction fragment length polymorphism identification (RFLPI), random amplified polymorphic detection (RAPD), amplified fragment length polymorphism detection (AFLPD), polymerase chain reaction (PCR), allele specific oligonucleotide (ASO) probes, and hybridization to DNA microarrays or beads. In some cases, the analyzing comprises performing structural analysis on the genomic region of interest.

In some cases, the analyzing comprises sequencing the genomic region of interest. In some cases, the sequencing is a long-read sequencing method (e.g., a third generation sequencing method). The long-read sequencing method may be any sequencing method that is capable of generating sequencing reads that are substantially longer than short-read sequencing methods (e.g., second generation sequencing methods). In some cases, the long-read sequencing method is a sequencing method that is capable of generating sequencing reads of at least 10,000 kilobases. In some cases, the long-read sequencing method is single-molecule real time sequencing (e.g., SMRT sequencing, Pacific Biosciences). In some cases, the long-read sequencing method is nanopore sequencing (e.g., MinION, GridION, and PromethION, as developed by Oxford Nanopore Technologies). In some aspects, prior to the sequencing, the methods further involve ligating adapters (e.g., sequencing adapters) to the ends of the genomic region of interest. The methods may, in some instances, involve any other processing methods suitable for sequencing applications, including, end-tailing steps, de-phosphorylation steps, and the like.

In various aspects, the methods provided herein are amplification-free (e.g., do not involve a nucleic acid amplification (e.g., DNA amplification) step). In some cases, the methods provided herein do not involve polymerase chain reaction (PCR). In some cases, the methods provided herein do not involve isothermal amplification. In some cases, the methods provided herein do not involve any one of loop mediated isothermal amplification (LAMP), nucleic acid sequence based amplification (NASBA), strand displacement amplification (SDA), multiple displacement amplification (MDA), rolling circle amplification (RCA), ligase chain reaction (LCR), helicase dependent amplification, and ramification amplification method (RAM). Nucleic acid amplification techniques often introduce errors into the Advantageously, the methods provided herein avoid the use of nucleic acid amplification methods which may introduce errors into the sequencing template.

In various aspects, the methods do not involve fragmenting, shearing, or digesting the genomic DNA. In some cases, the methods do not involve digesting the genomic DNA with, e.g., restriction enzymes. In other words, the methods are performed directly on genomic DNA that has not been sheared, digested, or fragmented. In other cases, the methods involve digestion with an exonuclease (e.g., after genomic DNA is contacted with the CRISPR-associated endonuclease and the outer pair of gRNAs, e.g., to remove background genomic DNA, as described herein).

In various aspects, the complex genomic region comprises a target gene, and one or more pseudogenes having high sequence identity to the target gene. In some cases, the one or more pseudogenes may have at least about 75% (e.g., at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%) sequence identity to the target gene. In one particular aspect, the genetic locus comprises the target gene CYP2D6, and the pseudogenes CYP2D7 and CYP2D8.

In various aspects, the complex genomic region comprises a target gene and one or more additional genes having high sequence identity to the target gene. In some cases, the one or more additional genes may have at least about 75% (e.g., at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%) sequence identity to the target gene. In one particular aspect, the genetic locus comprises the genes CYP2C8, CYP2C9, CYP2C18, and CYP2C19. In some cases, the genetic locus is generally difficult or challenging to sequence accurately by traditional methods (e.g., by short-read sequencing methods).

In various aspects, the complex genomic region is a highly polymorphic genetic locus. In various aspects, the complex genomic region comprises one or more repetitive regions, one or more duplications, one or more insertions, one or more inversions, one or more tandem repeats, one or more retrotransposons, or any combination thereof.

In some cases, the complex genomic region of interest is at least about 10 kilobases in length. For example, the genomic region of interest may be at least about 10 kilobases in length, at least about 15 kilobases in length, at least about 20 kilobases in length, at least about 25 kilobases in length, at least about 30 kilobases in length, at least about 35 kilobases in length, at least about 40 kilobases in length, at least about 45 kilobases in length, at least about 50 kilobases in length, at least about 55 kilobases in length, at least about 60 kilobases in length, at least about 65 kilobases in length, at least about 70 kilobases in length, at least about 75 kilobases in length, at least about 80 kilobases in length, at least about 85 kilobases in length, at least about 90 kilobases in length, at least about 95 kilobases in length, at least about 100 kilobases in length, at least about 110 kilobases in length, at least about 120 kilobases in length, at least about 130 kilobases in length, at least about 140 kilobases in length, at least about 150 kilobases in length, at least about 160 kilobases in length, at least about 170 kilobases in length, at least about 180 kilobases in length, at least about 190 kilobases in length, at least about 200 kilobases in length, at least about 210 kilobases in length, at least about 220 kilobases in length, at least about 230 kilobases in length, at least about 240 kilobases in length, or at least about 250 kilobases in length. In some aspects, the genomic region of interest is greater than about 10 kilobases in length. In some aspects, the genomic region of interest is less than about 250 kilobases in length.

In some cases, at least one of the gRNAs (e.g., at least one of the first outer gRNA, the second outer gRNA, the first inner gRNA, and the second inner gRNA) comprises a nucleotide sequence according to any nucleotide sequence provided below in Table 1 (e.g., SEQ ID NOs: 1-418). In some cases, at least one of the gRNAs (e.g., at least one of the first outer gRNA, the second outer gRNA, the first inner gRNA, and the second inner gRNA) comprises a nucleotide sequence having at least about 90% (e.g., at least about 91%, at least about 92%, at least about 93%, at least about 94%, at least about 95%, at least about 96%, at least about 97%, at least about 98%, at least about 99%) sequence identity to any nucleotide sequence provided below in Table 1 (e.g., SEQ ID NOs: 1-418). In some embodiments, for a pair of gRNAs, a first gRNA is selected such that it is complementary or substantially complementary to a nucleotide sequence present on genomic DNA that is upstream of CYP2D6, and a second gRNA is selected such that it is complementary or substantially complementary to a nucleotide sequence present on genomic DNA that is downstream of CYP2D8. Table 1 provides a non-limiting list of gRNAs that may be used in the present disclosure (e.g., to excise a fragment of genomic DNA containing the entire CYP2D6 locus), along with location relative to the CYP2D6 locus (e.g., upstream of CYP2D6 or downstream of CYP2D8). In some cases, a first gRNA comprises a nucleotide sequence of any one of SEQ ID NOS: 1, 2, 13-16, 27-67, 78-81, and 215-343, or a nucleotide sequence having at least 90% sequence identity (e.g., at least about 91%, at least about 92%, at least about 93%, at least about 94%, at least about 95%, at least about 96%, at least about 97%, at least about 98%, at least about 99%) to any one of SEQ ID NOS: 1, 2, 13-16, 27-67, 78-81, and 215-343. In some cases, a second gRNA comprises a nucleotide sequence of any one of SEQ ID NOS: 3-12, 17-26, 68-77, 82-214, 344-418, or a nucleotide sequence having at least 90% sequence identity (e.g., at least about 91%, at least about 92%, at least about 93%, at least about 94%, at least about 95%, at least about 96%, at least about 97%, at least about 98%, at least about 99%) to any one of SEQ ID NOS: 3-12, 17-26, 68-77, 82-214, and 344-418. In some cases, at least one of the gRNAs is a crRNA. In some cases, at least one of the gRNAs is an sgRNA.

TABLE_1
Guide RNA sequences

| gRNA | Location | SEQ ID NO | Sequence |
|---|---|---|---|
| TCF20_1_1 | downstream | 1 | AAGGUGGUGGACACUCGUGAGUUUUAGAGC |
| | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_2_1 | downstream | 2 | CACUAUGGAGAUUGUGUCCAGUUUUAGAGC |
| | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6_D6_1 | upstream of | 3 | ACGGACACUACCAAGGAGCGGUUUUAGAGC |
| | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6_D6_2 | upstream of | 4 | CUUGAAGAACCUCCUCGUGGGUUUUAGAGC |
| | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| N3 | upstream of | 5 | AUGUCUCAAGACUACCCCUCGUUUUAGAGC |
| | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| AD6_C | upstream of | 6 | CUGUCAUGGGCACGUAGACCGUUUUAGAGC |
| | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| AD6_D | upstream of | 7 | UCCUCACCGACAUAAUGGGCGUUUUAGAGC |
| | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| JGYW3632.AA | upstream of | 8 | GGCUUACAAGUUGGUCCUAAGUUUUAGAGC |
| | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| BJGYW3632.AB | upstream of | 9 | UAUCACCUUUUAGUCAAUUCGUUUUAGAGC |
| | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| AD6_E | upstream of | 10 | UGUCAAGAAUUAGUGGUGGUGUUUUAGAG |
| | CYP2D6 | | CUAGAAAUAGCAAGUUAAAAUAAGGCUAG |
| | | | UCCGUUAUCAACUUGAAAAAGUGGCACCGA |
| | | | GUCGGUGCUUUU |
| N4 | upstream of | 11 | CCAUUCACCCUUAUGCUCAGGUUUUAGAGC |
| | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| N5 | upstream of | 12 | AACCUCCGGUUGCUUCCUGAGUUUUAGAGC |
| | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| T3 | downstream | 13 | GGUGGACACUCGUGAUGGAAGUUUUAGAGC |
| | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| T3_2 | downstream | 14 | GGUGGACACUCGUGAUGGAAGUUUUAGAGC |
| | of CYP2D8 | | UAUGCU |
| TCF20_1_2 | downstream | 15 | AAGGUGGUGGACACUCGUGAGUUUUAGAGC |
| | of CYP2D8 | | UAUGCU |
| TCF20_2_2 | downstream | 16 | CACUAUGGAGAUUGUGUCCAGUUUUAGAGC |
| | of CYP2D8 | | UAUGCU |
| NDUFA6_D6_1_2 | upstream of | 17 | ACGGACACUACCAAGGAGCGGUUUUAGAGC |
| | CYP2D6 | | UAUGCU |
| NDUFA6_D6_2_2 | upstream of | 18 | CUUGAAGAACCUCCUCGUGGGUUUUAGAGC |
| | CYP2D6 | | UAUGCU |
| N3_2 | upstream of | 19 | AUGUCUCAAGACUACCCCUCGUUUUAGAGC |
| | CYP2D6 | | UAUGCU |
| AD6_C_2 | upstream of | 20 | CUGUCAUGGGCACGUAGACCGUUUUAGAGC |
| | CYP2D6 | | UAUGCU |
| AD6_D_2 | upstream of | 21 | UCCUCACCGACAUAAUGGGCGUUUUAGAGC |
| | CYP2D6 | | UAUGCU |
| JGYW3632.AA_2 | upstream of | 22 | GGCUUACAAGUUGGUCCUAAGUUUUAGAGC |
| | CYP2D6 | | UAUGCU |
| BJGYW3632.AB_2 | upstream of | 23 | UAUCACCUUUUAGUCAAUUCGUUUUAGAGC |
| | CYP2D6 | | UAUGCU |
| AD6_E_2 | upstream of | 24 | UGUCAAGAAUUAGUGGUGGUGUUUUAGAG |
| | CYP2D6 | | CUAUGCU |
| N4_2 | upstream of | 25 | CCAUUCACCCUUAUGCUCAGGUUUUAGAGC |
| | CYP2D6 | | UAUGCU |
| N5_2 | upstream of | 26 | AACCUCCGGUUGCUUCCUGAGUUUUAGAGC |
| | CYP2D6 | | UAUGCU |
| TCF20-1 | downstream | 27 | UGGUCCAUGUUUUCAAGAGU |
| | of CYP2D8 | | |
| TCF20-2 | downstream | 28 | ACUCAAACCAGUGACACCAC |
| | of CYP2D8 | | |
| TCF20-3 | downstream | 29 | AAAGACCCAAGACGUUGGAA |
| | of CYP2D8 | | |
| TCF20-4 | downstream | 30 | GUUCAGAAAACACUAGACCC |
| | of CYP2D8 | | |
| TCF20-5 | downstream | 31 | GGGUCUAGUGUUUUCUGAAC |
| | of CYP2D8 | | |
| TCF20-6 | downstream | 32 | ACCCUCAUCUCAUGAAGGAC |
| | of CYP2D8 | | |
| TCF20-7 | downstream | 33 | ACUUGUCAUCGGAACAAAUU |
| | of CYP2D8 | | |
| TCF20-8 | downstream | 34 | CUCCCCCCACAUUGUCACUA |
| | of CYP2D8 | | |
| TCF20-9 | downstream | 35 | CCAGGGGUACCACGGAACAG |
| | of CYP2D8 | | |
| TCF20-10 | downstream | 36 | CCCUCAUCUCAUGAAGGACG |
| | of CYP2D8 | | |
| TCF20-11 | downstream | 37 | ACACACCCGAGACCAAUGCC |
| | of CYP2D8 | | |
| TCF20-12 | downstream | 38 | AACAGCCAUUCCAACGUCUU |
| | of CYP2D8 | | |
| TCF20-13 | downstream | 39 | UACCACGGAACAGCGGCUGU |
| | of CYP2D8 | | |
| TCF20-14 | downstream | 40 | UGGUCCAUGUUUUCAAGAGUGUUUAGAGCU |
| | of CYP2D8 | | AUGCU |
| TCF20-15 | downstream | 41 | ACUCAAACCAGUGACACCACGUUUAGAGCU |
| | of CYP2D8 | | AUGCU |
| TCF20-16 | downstream | 42 | AAAGACCCAAGACGUUGGAAGUUUAGAGCU |
| | of CYP2D8 | | AUGCU |
| TCF20-17 | downstream | 43 | GUUCAGAAAACACUAGACCCGUUUAGAGCU |
| | of CYP2D8 | | AUGCU |
| TCF20-18 | downstream | 44 | GGGUCUAGUGUUUUCUGAACGUUUAGAGCU |
| | of CYP2D8 | | AUGCU |
| TCF20-19 | downstream | 45 | ACCCUCAUCUCAUGAAGGACGUUUAGAGCU |
| | of CYP2D8 | | AUGCU |
| TCF20-20 | downstream | 46 | ACUUGUCAUCGGAACAAAUUGUUUAGAGCU |
| | of CYP2D8 | | AUGCU |
| TCF20-21 | downstream | 47 | CUCCCCCCACAUUGUCACUAGUUUAGAGCU |
| | of CYP2D8 | | AUGCU |
| TCF20-22 | downstream | 48 | CCAGGGGUACCACGGAACAGGUUUAGAGCU |
| | of CYP2D8 | | AUGCU |
| TCF20-23 | downstream | 49 | CCCUCAUCUCAUGAAGGACGGUUUAGAGCU |
| | of CYP2D8 | | AUGCU |
| TCF20-24 | downstream | 50 | ACACACCCGAGACCAAUGCCGUUUAGAGCU |
| | of CYP2D8 | | AUGCU |
| TCF20-25 | downstream | 51 | AACAGCCAUUCCAACGUCUUGUUUAGAGCU |
| | of CYP2D8 | | AUGCU |
| TCF20-26 | downstream | 52 | UACCACGGAACAGCGGCUGUGUUUAGAGCU |
| | of CYP2D8 | | AUGCU |
| TCF20-27 | downstream | 53 | UGGUCCAUGUUUUCAAGAGUGUUUUAGAGC |
| | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20-28 | downstream | 54 | ACUCAAACCAGUGACACCACGUUUUAGAGC |
| | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20-29 | downstream | 55 | AAAGACCCAAGACGUUGGAAGUUUUAGAGC |
| | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20-30 | downstream | 56 | GUUCAGAAAACACUAGACCCGUUUUAGAGC |
| | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20-31 | downstream | 57 | GGGUCUAGUGUUUUCUGAACGUUUUAGAGC |
| | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20-32 | downstream | 58 | ACCCUCAUCUCAUGAAGGACGUUUUAGAGC |
| | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20-33 | downstream | 59 | ACUUGUCAUCGGAACAAAUUGUUUUAGAGC |
| | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20-34 | downstream | 60 | CUCCCCCCACAUUGUCACUAGUUUUAGAGC |
| | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20-35 | downstream | 61 | CCAGGGGUACCACGGAACAGGUUUUAGAGC |
| | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20-36 | downstream | 62 | CCCUCAUCUCAUGAAGGACGGUUUUAGAGC |
| | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20-37 | downstream | 63 | ACACACCCGAGACCAAUGCCGUUUUAGAGC |
| | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20-38 | downstream | 64 | AACAGCCAUUCCAACGUCUUGUUUUAGAGC |
| | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20-39 | downstream | 65 | UACCACGGAACAGCGGCUGUGUUUUAGAGC |
| | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_1_1 1: | downstream | 66 | AAGGUGGUGGACACUCGUGA |
| | of CYP2D8 | | |
| TCF20_2_1 2: | downstream | 67 | CACUAUGGAGAUUGUGUCCA |
| | of CYP2D8 | | |
| NDUFA6_D6_1 3: | upstream of | 68 | ACGGACACUACCAAGGAGCG |
| | CYP2D6 | | |
| NDUFA6_D6_2 4: | upstream of | 69 | CUUGAAGAACCUCCUCGUGG |
| | CYP2D6 | | |
| N3 5: | upstream of | 70 | AUGUCUCAAGACUACCCCUC |
| | CYP2D6 | | |
| AD6_C 6: | upstream of | 71 | CUGUCAUGGGCACGUAGACC |
| | CYP2D6 | | |
| AD6_D 7: | upstream of | 72 | UCCUCACCGACAUAAUGGGC |
| | CYP2D6 | | |
| JGYW3632.A | upstream of | 73 | GGCUUACAAGUUGGUCCUAA |
| A8: | CYP2D6 | | |
| BJGYW3632.AB 9: | upstream of | 74 | UAUCACCUUUUAGUCAAUUC |
| | CYP2D6 | | |
| AD6_E 10: | upstream of | 75 | UGUCAAGAAUUAGUGGUGGU |
| | CYP2D6 | | |
| N4 11: C | upstream of | 76 | CAUUCACCCUUAUGCUCAG |
| | CYP2D6 | | |
| N5 12: | upstream of | 77 | AACCUCCGGUUGCUUCCUGA |
| | CYP2D6 | | |
| T3 13: | downstream | 78 | GGUGGACACUCGUGAUGGAA |
| | of CYP2D8 | | |
| T3_2 14: | downstream | 79 | GGUGGACACUCGUGAUGGAA |
| | of CYP2D8 | | |
| TCF20_1_2 15: | downstream | 80 | AAGGUGGUGGACACUCGUGA |
| | of CYP2D8 | | |
| TCF20_2_2 16: | downstream | 81 | CACUAUGGAGAUUGUGUCCA |
| | of CYP2D8 | | |
| NDUFA6_D6 | upstream of | 82 | ACGGACACUACCAAGGAGCG |
| 1_2 17: | CYP2D6 | | |
| NDUFA6_D6 | upstream of | 83 | CUUGAAGAACCUCCUCGUGG |
| 2_2 18: | CYP2D6 | | |
| N3_2 19: | upstream of | 84 | AUGUCUCAAGACUACCCCUC |
| | CYP2D6 | | |
| AD6_C_2 20: | upstream of | 85 | CUGUCAUGGGCACGUAGACC |
| | CYP2D6 | | |
| AD6_D_2 21: | upstream of | 86 | UCCUCACCGACAUAAUGGGC |
| | CYP2D6 | | |
| JGYW3632.A | upstream of | 87 | GGCUUACAAGUUGGUCCUAA |
| A_2 22: | CYP2D6 | | |
| BJGYW3632. | upstream of | 88 | UAUCACCUUUUAGUCAAUUC |
| AB_2 23: | CYP2D6 | | |
| AD6 E_2 24: | upstream of | 89 | UGUCAAGAAUUAGUGGUGGU |
| | CYP2D6 | | |
| N4_2 25: | upstream of | 90 | CCAUUCACCCUUAUGCUCAG |
| | CYP2D6 | | |
| N5_2 26: | upstream of | 91 | AACCUCCGGUUGCUUCCUGA |
| | CYP2D6 | | |
| NDUFA6- | upstream of | 92 | GAGGUCACCAACUUGGGCAG |
| after D6-1 | CYP2D6 | | |
| NDUFA6- | upstream of | 93 | CCCAAGUUGGUGACCUCAGC |
| after D6-2 | CYP2D6 | | |
| NDUFA6- | upstream of | 94 | CCAGCUGAGGUCACCAACUU |
| after D6-3 | CYP2D6 | | |
| NDUFA6- | upstream of | 95 | AGGUGCCGAACACUGGUGAG |
| after D6-4 | CYP2D6 | | |
| NDUFA6- | upstream of | 96 | GGACCCCGAGGUAACUGCUG |
| after D6-5 | CYP2D6 | | |
| NDUFA6- | upstream of | 97 | GGCCUUGAAGAACCUCCUCG |
| after D6-6 | CYP2D6 | | |
| NDUFA6- | upstream of | 98 | UGACUCUGAGGCUCUCGGAU |
| after D6-7 | CYP2D6 | | |
| NDUFA6- | upstream of | 99 | UCGUGAAGCCCAUUUUCAGU |
| after D6-8 | CYP2D6 | | |
| NDUFA6- | upstream of | 100 | ACUGAAAAUGGGCUUCACGA |
| after D6-9 | CYP2D6 | | |
| NDUFA6- | upstream of | 101 | CACGACCCAGCGACCUCCUG |
| after D6-10 | CYP2D6 | | |
| NDUFA6- | upstream of | 102 | GAUGCUUUGGCAAGAUGGCG |
| after D6-11 | CYP2D6 | | |
| NDUFA6- | upstream of | 103 | UUGAAGAACCUCCUCGUGGC |
| after D6-12 | CYP2D6 | | |
| NDUFA6- | upstream of | 104 | ACAUGAACGAGGCCAAGCGG |
| after D6-13 | CYP2D6 | | |
| NDUFA6- | upstream of | 105 | CAUGAACGAGGCCAAGCGGA |
| after D6-14 | CYP2D6 | | |
| NDUFA6- | upstream of | 106 | CGACAGAUGGUGUAGUCCAA |
| after D6-15 | CYP2D6 | | |
| NDUFA6- | upstream of | 107 | CUUGAAGAACCUCCUCGUGG |
| after D6-16 | CYP2D6 | | |
| NDUFA6- | upstream of | 108 | AAUGGGCUUCACGAAGGUGC |
| after D6-17 | CYP2D6 | | |
| NDUFA6- | upstream of | 109 | GAAUGUCCCUGUCUACGAUG |
| after D6-18 | CYP2D6 | | |
| NDUFA6- | upstream of | 110 | AGGGUCACCCGAGCCUACCA |
| after D6-19 | CYP2D6 | | |
| NDUFA6- | upstream of | 111 | ACGGACACUACCAAGGAGCG |
| after D6-20 | CYP2D6 | | |
| NDUFA6- | upstream of | 112 | GACACUACCAAGGAGCGCGG |
| after D6-21 | CYP2D6 | | |
| NDUFA6- | upstream of | 113 | UUUCAGUCGGGACAUGAACG |
| after D6-22 | CYP2D6 | | |
| NDUFA6- | upstream of | 114 | ACACUACCAAGGAGCGCGGC |
| after D6-23 | CYP2D6 | | |
| NDUFA6- | upstream of | 115 | GGGUCACCCGAGCCUACCAU |
| after D6-24 | CYP2D6 | | |
| NDUFA6- | upstream of | 116 | UGAGAGGUAGCGGCUUACGU |
| after D6-25 | CYP2D6 | | |
| NDUFA6- | upstream of | 117 | GAGGUCACCAACUUGGGCAGGUUUAGAGCU |
| after D6-26 | CYP2D6 | | AUGCU |
| NDUFA6- | upstream of | 118 | CCCAAGUUGGUGACCUCAGCGUUUAGAGCU |
| after D6-27 | CYP2D6 | | AUGCU |
| NDUFA6- | upstream of | 119 | CCAGCUGAGGUCACCAACUUGUUUAGAGCU |
| after D6-28 | CYP2D6 | | AUGCU |
| NDUFA6- | upstream of | 120 | AGGUGCCGAACACUGGUGAGGUUUAGAGCU |
| after D6-29 | CYP2D6 | | AUGCU |
| NDUFA6- | upstream of | 121 | GGACCCCGAGGUAACUGCUGGUUUAGAGCU |
| after D6-30 | CYP2D6 | | AUGCU |
| NDUFA6- | upstream of | 122 | GGCCUUGAAGAACCUCCUCGGUUUAGAGCU |
| after D6-31 | CYP2D6 | | AUGCU |
| NDUFA6- | upstream of | 123 | UGACUCUGAGGCUCUCGGAUGUUUAGAGCU |
| after D6-32 | CYP2D6 | | AUGCU |
| NDUFA6- | upstream of | 124 | UCGUGAAGCCCAUUUUCAGUGUUUAGAGCU |
| after D6-33 | CYP2D6 | | AUGCU |
| NDUFA6- | upstream of | 125 | ACUGAAAAUGGGCUUCACGAGUUUAGAGCU |
| after D6-34 | CYP2D6 | | AUGCU |
| NDUFA6- | upstream of | 126 | CACGACCCAGCGACCUCCUGGUUUAGAGCU |
| after D6-35 | CYP2D6 | | AUGCU |
| NDUFA6- | upstream of | 127 | GAUGCUUUGGCAAGAUGGCGGUUUAGAGCU |
| after D6-36 | CYP2D6 | | AUGCU |
| NDUFA6- | upstream of | 128 | UUGAAGAACCUCCUCGUGGCGUUUAGAGCU |
| after D6-37 | CYP2D6 | | AUGCU |
| NDUFA6- | upstream of | 129 | ACAUGAACGAGGCCAAGCGGGUUUAGAGCU |
| after D6-38 | CYP2D6 | | AUGCU |
| NDUFA6- | upstream of | 130 | CAUGAACGAGGCCAAGCGGAGUUUAGAGCU |
| after D6-39 | CYP2D6 | | AUGCU |
| NDUFA6- | upstream of | 131 | CGACAGAUGGUGUAGUCCAAGUUUAGAGCU |
| after D6-40 | CYP2D6 | | AUGCU |
| NDUFA6- | upstream of | 132 | CUUGAAGAACCUCCUCGUGGGUUUAGAGCU |
| after D6-41 | CYP2D6 | | AUGCU |
| NDUFA6- | upstream of | 133 | AAUGGGCUUCACGAAGGUGCGUUUAGAGCU |
| after D6-42 | CYP2D6 | | AUGCU |
| NDUFA6- | upstream of | 134 | GAAUGUCCCUGUCUACGAUGGUUUAGAGCU |
| after D6-43 | CYP2D6 | | AUGCU |
| NDUFA6- | upstream of | 135 | AGGGUCACCCGAGCCUACCAGUUUAGAGCU |
| after D6-44 | CYP2D6 | | AUGCU |
| NDUFA6- | upstream of | 136 | ACGGACACUACCAAGGAGCGGUUUAGAGCU |
| after D6-45 | CYP2D6 | | AUGCU |
| NDUFA6- | upstream of | 137 | GACACUACCAAGGAGCGCGGGUUUAGAGCU |
| after D6-46 | CYP2D6 | | AUGCU |
| NDUFA6- | upstream of | 138 | UUUCAGUCGGGACAUGAACGGUUUAGAGCU |
| after D6-47 | CYP2D6 | | AUGCU |
| NDUFA6- | upstream of | 139 | ACACUACCAAGGAGCGCGGCGUUUAGAGCU |
| after D6-48 | CYP2D6 | | AUGCU |
| NDUFA6- | upstream of | 140 | GGGUCACCCGAGCCUACCAUGUUUAGAGCU |
| after D6-49 | CYP2D6 | | AUGCU |
| NDUFA6- | upstream of | 141 | UGAGAGGUAGCGGCUUACGUGUUUAGAGCU |
| after D6-50 | CYP2D6 | | AUGCU |
| NDUFA6- | upstream of | 142 | GAGGUCACCAACUUGGGCAGGUUUUAGAGC |
| after D6-51 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 143 | CCCAAGUUGGUGACCUCAGCGUUUUAGAGC |
| after D6-52 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 144 | CCAGCUGAGGUCACCAACUUGUUUUAGAGC |
| after D6-53 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 145 | AGGUGCCGAACACUGGUGAGGUUUUAGAGC |
| after D6-54 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 146 | GGACCCCGAGGUAACUGCUGGUUUUAGAGC |
| after D6-55 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 147 | GGCCUUGAAGAACCUCCUCGGUUUUAGAGC |
| after D6-56 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 148 | UGACUCUGAGGCUCUCGGAUGUUUUAGAGC |
| after D6-57 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 149 | UCGUGAAGCCCAUUUUCAGUGUUUUAGAGC |
| after D6-58 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 150 | ACUGAAAAUGGGCUUCACGAGUUUUAGAGC |
| after D6-59 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 151 | CACGACCCAGCGACCUCCUGGUUUUAGAGC |
| after D6-60 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 152 | GAUGCUUUGGCAAGAUGGCGGUUUUAGAGC |
| after D6-61 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 153 | UUGAAGAACCUCCUCGUGGCGUUUUAGAGC |
| after D6-62 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 154 | ACAUGAACGAGGCCAAGCGGGUUUUAGAGC |
| after D6-63 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 155 | CAUGAACGAGGCCAAGCGGAGUUUUAGAGC |
| after D6-64 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 156 | CGACAGAUGGUGUAGUCCAAGUUUUAGAGC |
| after D6-65 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 157 | CUUGAAGAACCUCCUCGUGGGUUUUAGAGC |
| after D6-66 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 158 | AAUGGGCUUCACGAAGGUGCGUUUUAGAGC |
| after D6-67 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 159 | GAAUGUCCCUGUCUACGAUGGUUUUAGAGC |
| after D6-68 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 160 | AGGGUCACCCGAGCCUACCAGUUUUAGAGC |
| after D6-69 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 161 | ACGGACACUACCAAGGAGCGGUUUUAGAGC |
| after D6-70 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 162 | GACACUACCAAGGAGCGCGGGUUUUAGAGC |
| after D6-71 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 163 | UUUCAGUCGGGACAUGAACGGUUUUAGAGC |
| after D6-72 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 164 | ACACUACCAAGGAGCGCGGCGUUUUAGAGC |
| after D6-73 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 165 | GGGUCACCCGAGCCUACCAUGUUUUAGAGC |
| after D6-74 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 166 | UGAGAGGUAGCGGCUUACGUGUUUUAGAGC |
| after D6-75 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 167 | UUAAUGCUAGAAUUAGGCAC |
| after D6_3-1 | CYP2D6 | | |
| NDUFA6- | upstream of | 168 | UUAGGCACAGGCUUACAAGU |
| after D6_3-2 | CYP2D6 | | |
| NDUFA6- | upstream of | 169 | GAAGUGGCCUGCCCUUCAAA |
| after D6_3-3 | CYP2D6 | | |
| NDUFA6- | upstream of | 170 | GGCUUACAAGUUGGUCCUAA |
| after D6_3-4 | CYP2D6 | | |
| NDUFA6- | upstream of | 171 | UUAAUGCUAGAAUUAGGCACGUUUAGAGCU |
| after D6_3-5 | CYP2D6 | | AUGCU |
| NDUFA6- | upstream of | 172 | UUAGGCACAGGCUUACAAGUGUUUAGAGCU |
| after D6_3-6 | CYP2D6 | | AUGCU |
| NDUFA6- | upstream of | 173 | GAAGUGGCCUGCCCUUCAAAGUUUAGAGCU |
| after D6_3-7 | CYP2D6 | | AUGCU |
| NDUFA6- | upstream of | 174 | GGCUUACAAGUUGGUCCUAAGUUUAGAGCU |
| after D6_3-8 | CYP2D6 | | AUGCU |
| NDUFA6- | upstream of | 175 | UUAAUGCUAGAAUUAGGCACGUUUUAGAGC |
| after D6_3-9 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 176 | UUAGGCACAGGCUUACAAGUGUUUUAGAGC |
| after D6_3-10 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 177 | GAAGUGGCCUGCCCUUCAAAGUUUUAGAGC |
| after D6_3-11 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 178 | GGCUUACAAGUUGGUCCUAAGUUUUAGAGC |
| after D6_3-12 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 179 | CUAAACAACAAUUUAGCUGU |
| after D6_2-1 | CYP2D6 | | |
| NDUFA6- | upstream of | 180 | CUAAACAACAAUUUAGCUGUGUUUAGAGCU |
| after D6_2-2 | CYP2D6 | | AUGCU |
| NDUFA6- | upstream of | 181 | CUAAACAACAAUUUAGCUGUGUUUUAGAGC |
| after D6_2-3 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 182 | CUUCACGGUUCUGAGUCUUG |
| after D6_1-1 | CYP2D6 | | |
| NDUFA6- | upstream of | 183 | ACCGAGCCGUGUGACCACAG |
| after D6_1-2 | CYP2D6 | | |
| NDUFA6- | upstream of | 184 | UCUGUCCUCACCGACAUAAU |
| after D6_1-3 | CYP2D6 | | |
| NDUFA6- | upstream of | 185 | AGGUGAAGCAGCCUUCUCGU |
| after D6_1-4 | CYP2D6 | | |
| NDUFA6- | upstream of | 186 | UCUGACUGACUCGGUGCCAG |
| after D6_1-5 | CYP2D6 | | |
| NDUFA6- | upstream of | 187 | UUCUGACUGACUCGGUGCCA |
| after D6_1-6 | CYP2D6 | | |
| NDUFA6- | upstream of | 188 | ACUGUGGUCACACGGCUCGG |
| after D6_1-7 | CYP2D6 | | |
| NDUFA6- | upstream of | 189 | UUCCCUAAGAAGGUCUGCCC |
| after D6_1-8 | CYP2D6 | | |
| NDUFA6- | upstream of | 190 | GUCUGUCCUCACCGACAUAA |
| after D6_1-9 | CYP2D6 | | |
| NDUFA6- | upstream of | 191 | CCUCACCGACAUAAUGGGCU |
| after D6_1-10 | CYP2D6 | | |
| NDUFA6- | upstream of | 192 | GGCACGUAGACCCGGUCCCA |
| after D6_1-11 | CYP2D6 | | |
| NDUFA6- | upstream of | 193 | CUUCACGGUUCUGAGUCUUGGUUUAGAGCU |
| after D6_1-12 | CYP2D6 | | AUGCU |
| NDUFA6- | upstream of | 194 | ACCGAGCCGUGUGACCACAGGUUUAGAGCU |
| after D6_1-13 | CYP2D6 | | AUGCU |
| NDUFA6- | upstream of | 195 | UCUGUCCUCACCGACAUAAUGUUUAGAGCU |
| after D6_1-14 | CYP2D6 | | AUGCU |
| NDUFA6- | upstream of | 196 | AGGUGAAGCAGCCUUCUCGUGUUUAGAGCU |
| after D6_1-15 | CYP2D6 | | AUGCU |
| NDUFA6- | upstream of | 197 | UCUGACUGACUCGGUGCCAGGUUUAGAGCU |
| after D6_1-16 | CYP2D6 | | AUGCU |
| NDUFA6- | upstream of | 198 | UUCUGACUGACUCGGUGCCAGUUUAGAGCU |
| after D6_1-17 | CYP2D6 | | AUGCU |
| NDUFA6- | upstream of | 199 | ACUGUGGUCACACGGCUCGGGUUUAGAGCU |
| after D6_1-18 | CYP2D6 | | AUGCU |
| NDUFA6- | upstream of | 200 | UUCCCUAAGAAGGUCUGCCCGUUUAGAGCU |
| after D6_1-19 | CYP2D6 | | AUGCU |
| NDUFA6- | upstream of | 201 | GUCUGUCCUCACCGACAUAAGUUUAGAGCU |
| after D6_1-20 | CYP2D6 | | AUGCU |
| NDUFA6- | upstream of | 202 | CCUCACCGACAUAAUGGGCUGUUUAGAGCU |
| after D6_1-21 | CYP2D6 | | AUGCU |
| NDUFA6- | upstream of | 203 | GGCACGUAGACCCGGUCCCAGUUUAGAGCU |
| after D6_1-22 | CYP2D6 | | AUGCU |
| NDUFA6- | upstream of | 204 | CUUCACGGUUCUGAGUCUUGGUUUUAGAGC |
| after D6_1-23 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 205 | ACCGAGCCGUGUGACCACAGGUUUUAGAGC |
| after D6_1-24 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 206 | UCUGUCCUCACCGACAUAAUGUUUUAGAGC |
| after D6_1-25 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 207 | AGGUGAAGCAGCCUUCUCGUGUUUUAGAGC |
| after D6_1-26 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 208 | UCUGACUGACUCGGUGCCAGGUUUUAGAGC |
| after D6_1-27 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 209 | UUCUGACUGACUCGGUGCCAGUUUUAGAGC |
| after D6_1-28 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 210 | ACUGUGGUCACACGGCUCGGGUUUUAGAGC |
| after D6_1-29 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 211 | UUCCCUAAGAAGGUCUGCCCGUUUUAGAGC |
| after D6_1-30 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 212 | GUCUGUCCUCACCGACAUAAGUUUUAGAGC |
| after D6_1-31 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 213 | CCUCACCGACAUAAUGGGCUGUUUUAGAGC |
| after D6_1-32 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA6- | upstream of | 214 | GGCACGUAGACCCGGUCCCAGUUUUAGAGC |
| after D6_1-33 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_10 kb- | downstream | 215 | UAUUAAUGGUCCAUCACAGC |
| 1 | of CYP2D8 | | |
| TCF20_10 kb- | downstream | 216 | GGAAGCACAAUUCACGUUCC |
| 2 | of CYP2D8 | | |
| TCF20_10 kb- | downstream | 217 | CUCACUGGUAUAAACCCCUG |
| 3 | of CYP2D8 | | |
| TCF20_10 kb- | downstream | 218 | GCACAAUUCACGUUCCUGGC |
| 4 | of CYP2D8 | | |
| TCF20_10 kb- | downstream | 219 | AGGGACCACACGAGCAGCAA |
| 5 | of CYP2D8 | | |
| TCF20_10 kb- | downstream | 220 | GGGUUUAUACCAGUGAGGAC |
| 6 | of CYP2D8 | | |
| TCF20_10 kb- | downstream | 221 | UCUGACAAGGCCUCCCAUGC |
| 7 | of CYP2D8 | | |
| TCF20_10 kb- | downstream | 222 | ACGUGAAUUGUGCUUCCUGA |
| 8 | of CYP2D8 | | |
| TCF20_10 kb- | downstream | 223 | ACAAUUCACGUUCCUGGCAG |
| 9 | of CYP2D8 | | |
| TCF20_10 kb- | downstream | 224 | GGAACGCAUUUCCUAACAUG |
| 10 | of CYP2D8 | | |
| TCF20_10 kb- | downstream | 225 | AUUGAGAGACCUUGACUGGC |
| 11 | of CYP2D8 | | |
| TCF20_10 kb- | downstream | 226 | CUGUUCUCAUACAUGUCCAC |
| 12 | of CYP2D8 | | |
| TCF20_10 kb- | downstream | 227 | CACAAUUCACGUUCCUGGCA |
| 13 | of CYP2D8 | | |
| TCF20_10 kb- | downstream | 228 | CAUGAGGCGUGUUUUAUUAA |
| 14 | of CYP2D8 | | |
| TCF20_10 kb- | downstream | 229 | CCUUGACUGGCUGGCCAUGU |
| 15 | of CYP2D8 | | |
| TCF20_10 kb- | downstream | 230 | UCUGGCAGCAAGCACUAUGC |
| 16 | of CYP2D8 | | |
| TCF20_10 kb- | downstream | 23 | AAACUAAUGCCAGAUACAUC |
| 17 | of CYP2D8 | | |
| TCF20_10 kb- | downstream | 232 | UAUUAAUGGUCCAUCACAGCGUUUAGAGCU |
| 18 | of CYP2D8 | | AUGCU |
| TCF20_10 kb- | downstream | 233 | GGAAGCACAAUUCACGUUCCGUUUAGAGCU |
| 19 | of CYP2D8 | | AUGCU |
| TCF20_10 kb- | downstream | 234 | CUCACUGGUAUAAACCCCUGGUUUAGAGCU |
| 20 | of CYP2D8 | | AUGCU |
| TCF20_10 kb- | downstream | 235 | GCACAAUUCACGUUCCUGGCGUUUAGAGCU |
| 21 | of CYP2D8 | | AUGCU |
| TCF20_10 kb- | downstream | 236 | AGGGACCACACGAGCAGCAAGUUUAGAGCU |
| 22 | of CYP2D8 | | AUGCU |
| TCF20_10 kb- | downstream | 237 | GGGUUUAUACCAGUGAGGACGUUUAGAGCU |
| 23 | of CYP2D8 | | AUGCU |
| TCF20_10 kb- | downstream | 238 | UCUGACAAGGCCUCCCAUGCGUUUAGAGCU |
| 24 | of CYP2D8 | | AUGCU |
| TCF20_10 kb- | downstream | 239 | ACGUGAAUUGUGCUUCCUGAGUUUAGAGCU |
| 25 | of CYP2D8 | | AUGCU |
| TCF20_10 kb- | downstream | 240 | ACAAUUCACGUUCCUGGCAGGUUUAGAGCU |
| 26 | of CYP2D8 | | AUGCU |
| TCF20_10 kb- | downstream | 241 | GGAACGCAUUUCCUAACAUGGUUUAGAGCU |
| 27 | of CYP2D8 | | AUGCU |
| TCF20_10 kb- | downstream | 242 | AUUGAGAGACCUUGACUGGCGUUUAGAGCU |
| 28 | of CYP2D8 | | AUGCU |
| TCF20_10 kb- | downstream | 243 | CUGUUCUCAUACAUGUCCACGUUUAGAGCU |
| 29 | of CYP2D8 | | AUGCU |
| TCF20_10 kb- | downstream | 244 | CACAAUUCACGUUCCUGGCAGUUUAGAGCU |
| 30 | of CYP2D8 | | AUGCU |
| TCF20_10 kb- | downstream | 245 | CAUGAGGCGUGUUUUAUUAAGUUUAGAGC |
| 31 | of CYP2D8 | | UAUGCU |
| TCF20_10 kb- | downstream | 246 | CCUUGACUGGCUGGCCAUGUGUUUAGAGCU |
| 32 | of CYP2D8 | | AUGCU |
| TCF20_10 kb- | downstream | 247 | UCUGGCAGCAAGCACUAUGCGUUUAGAGCU |
| 33 | of CYP2D8 | | AUGCU |
| TCF20_10 kb- | downstream | 248 | AAACUAAUGCCAGAUACAUCGUUUAGAGCU |
| 34 | of CYP2D8 | | AUGCU |
| TCF20_10 kb- | downstream | 249 | UAUUAAUGGUCCAUCACAGCGUUUUAGAGC |
| 35 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_10 kb- | downstream | 250 | GGAAGCACAAUUCACGUUCCGUUUUAGAGC |
| 36 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_10 kb- | downstream | 251 | CUCACUGGUAUAAACCCCUGGUUUUAGAGC |
| 37 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_10 kb- | downstream | 252 | GCACAAUUCACGUUCCUGGCGUUUUAGAGC |
| 38 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_10 kb- | downstream | 253 | AGGGACCACACGAGCAGCAAGUUUUAGAGC |
| 39 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_10 kb- | downstream | 254 | GGGUUUAUACCAGUGAGGACGUUUUAGAGC |
| 40 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_10 kb- | downstream | 255 | UCUGACAAGGCCUCCCAUGCGUUUUAGAGC |
| 41 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_10 kb- | downstream | 256 | ACGUGAAUUGUGCUUCCUGAGUUUUAGAGC |
| 42 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_10 kb- | downstream | 257 | ACAAUUCACGUUCCUGGCAGGUUUUAGAGC |
| 43 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_10 kb- | downstream | 258 | GGAACGCAUUUCCUAACAUGGUUUUAGAGC |
| 44 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_10 kb- | downstream | 259 | AUUGAGAGACCUUGACUGGCGUUUUAGAGC |
| 45 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_10 kb- | downstream | 260 | CUGUUCUCAUACAUGUCCACGUUUUAGAGC |
| 46 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_10 kb- | downstream | 261 | CACAAUUCACGUUCCUGGCAGUUUUAGAGC |
| 47 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_10 kb- | downstream | 262 | CAUGAGGCGUGUUUUAUUAAGUUUUAGAG |
| 48 | of CYP2D8 | | CUAGAAAUAGCAAGUUAAAAUAAGGCUAG |
| | | | UCCGUUAUCAACUUGAAAAAGUGGCACCGA |
| | | | GUCGGUGCUUUU |
| TCF20_10 kb- | downstream | 263 | CCUUGACUGGCUGGCCAUGUGUUUUAGAGC |
| 49 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_10 kb- | downstream | 264 | UCUGGCAGCAAGCACUAUGCGUUUUAGAGC |
| 50 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_10 kb- | downstream | 265 | AAACUAAUGCCAGAUACAUCGUUUUAGAGC |
| 51 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_20 kb- | downstream | 266 | AUCCUUAGUAGGGUCACAUG |
| 1 | of CYP2D8 | | |
| TCF20_20 kb- | downstream | 267 | UGUGACCCUACUAAGGAUGC |
| 2 | of CYP2D8 | | |
| TCF20_20 kb- | downstream | 268 | ACACUCCUCCUUAUAUGGUC |
| 3 | of CYP2D8 | | |
| TCF20_20 kb- | downstream | 269 | ACGUGCUGAGGUCUAACAGA |
| 4 | of CYP2D8 | | |
| TCF20_20 kb- | downstream | 270 | AACCACAUGUGACCCUACUA |
| 5 | of CYP2D8 | | |
| TCF20_20 kb- | downstream | 271 | AAGAGCCAGCAUCCUUAGUA |
| 6 | of CYP2D8 | | |
| TCF20_20 kb- | downstream | 272 | GCACGUGUCUCUGUGGUUAG |
| 7 | of CYP2D8 | | |
| TCF20_20 kb- | downstream | 273 | UCUGUGGUUAGAGGAGUCCG |
| 8 | of CYP2D8 | | |
| TCF20_20 kb- | downstream | 274 | GUGGUUAGAGGAGUCCGUGG |
| 9 | of CYP2D8 | | |
| TCF20_20 kb- | downstream | 275 | UUGAGACACUCCUCCUUAUA |
| 10 | of CYP2D8 | | |
| TCF20_20 kb- | downstream | 276 | CUGUGAGUGCUCAUCCUGUC |
| 11 | of CYP2D8 | | |
| TCF20_20 kb- | downstream | 277 | CCAUUCACUGACCACACCAU |
| 12 | of CYP2D8 | | |
| TCF20_20 kb- | downstream | 278 | GUGCUGAGGUCUAACAGAUG |
| 13 | of CYP2D8 | | |
| TCF20_20 kb- | downstream | 279 | ACACAACCAGCAAGACUAGC |
| 14 | of CYP2D8 | | |
| TCF20_20 kb- | downstream | 280 | GGACACAUUUCUUACCUGAC |
| 15 | of CYP2D8 | | |
| TCF20_20 kb- | downstream | 281 | GAAGAGCCAGCAUCCUUAGU |
| 16 | of CYP2D8 | | |
| TCF20_20 kb- | downstream | 282 | AUCCUUAGUAGGGUCACAUGGUUUAGAGCU |
| 17 | of CYP2D8 | | AUGCU |
| TCF20_20 kb- | downstream | 283 | UGUGACCCUACUAAGGAUGCGUUUAGAGCU |
| 18 | of CYP2D8 | | AUGCU |
| TCF20_20 kb- | downstream | 284 | ACACUCCUCCUUAUAUGGUCGUUUAGAGCU |
| 19 | of CYP2D8 | | AUGCU |
| TCF20_20 kb- | downstream | 285 | ACGUGCUGAGGUCUAACAGAGUUUAGAGCU |
| 20 | of CYP2D8 | | AUGCU |
| TCF20_20 kb- | downstream | 286 | AACCACAUGUGACCCUACUAGUUUAGAGCU |
| 21 | of CYP2D8 | | AUGCU |
| TCF20_20 kb- | downstream | 287 | AAGAGCCAGCAUCCUUAGUAGUUUAGAGCU |
| 22 | of CYP2D8 | | AUGCU |
| TCF20_20 kb- | downstream | 288 | GCACGUGUCUCUGUGGUUAGGUUUAGAGCU |
| 23 | of CYP2D8 | | AUGCU |
| TCF20_20 kb- | downstream | 289 | UCUGUGGUUAGAGGAGUCCGGUUUAGAGCU |
| 24 | of CYP2D8 | | AUGCU |
| TCF20_20 kb- | downstream | 290 | GUGGUUAGAGGAGUCCGUGGGUUUAGAGC |
| 25 | of CYP2D8 | | UAUGCU |
| TCF20_20 kb- | downstream | 291 | UUGAGACACUCCUCCUUAUAGUUUAGAGCU |
| 26 | of CYP2D8 | | AUGCU |
| TCF20_20 kb- | downstream | 292 | CUGUGAGUGCUCAUCCUGUCGUUUAGAGCU |
| 27 | of CYP2D8 | | AUGCU |
| TCF20_20 kb- | downstream | 293 | CCAUUCACUGACCACACCAUGUUUAGAGCU |
| 28 | of CYP2D8 | | AUGCU |
| TCF20_20 kb- | downstream | 294 | GUGCUGAGGUCUAACAGAUGGUUUAGAGCU |
| 29 | of CYP2D8 | | AUGCU |
| TCF20_20 kb- | downstream | 295 | ACACAACCAGCAAGACUAGCGUUUAGAGCU |
| 30 | of CYP2D8 | | AUGCU |
| TCF20_20 kb- | downstream | 296 | GGACACAUUUCUUACCUGACGUUUAGAGCU |
| 31 | of CYP2D8 | | AUGCU |
| TCF20_20 kb- | downstream | 297 | GAAGAGCCAGCAUCCUUAGUGUUUAGAGCU |
| 32 | of CYP2D8 | | AUGCU |
| TCF20_20 kb- | downstream | 298 | AUCCUUAGUAGGGUCACAUGGUUUUAGAGC |
| 33 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_20 kb- | downstream | 299 | UGUGACCCUACUAAGGAUGCGUUUUAGAGC |
| 34 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_20 kb- | downstream | 300 | ACACUCCUCCUUAUAUGGUCGUUUUAGAGC |
| 35 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_20 kb- | downstream | 301 | ACGUGCUGAGGUCUAACAGAGUUUUAGAGC |
| 36 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_20 kb- | downstream | 302 | AACCACAUGUGACCCUACUAGUUUUAGAGC |
| 37 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_20 kb- | downstream | 303 | AAGAGCCAGCAUCCUUAGUAGUUUUAGAGC |
| 38 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_20 kb- | downstream | 304 | GCACGUGUCUCUGUGGUUAGGUUUUAGAGC |
| 39 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_20 kb- | downstream | 305 | UCUGUGGUUAGAGGAGUCCGGUUUUAGAGC |
| 40 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_20 kb- | downstream | 306 | GUGGUUAGAGGAGUCCGUGGGUUUUAGAG |
| 41 | of CYP2D8 | | CUAGAAAUAGCAAGUUAAAAUAAGGCUAG |
| | | | UCCGUUAUCAACUUGAAAAAGUGGCACCGA |
| | | | GUCGGUGCUUUU |
| TCF20_20 kb- | downstream | 307 | UUGAGACACUCCUCCUUAUAGUUUUAGAGC |
| 42 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_20 kb- | downstream | 308 | CUGUGAGUGCUCAUCCUGUCGUUUUAGAGC |
| 43 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_20 kb- | downstream | 309 | CCAUUCACUGACCACACCAUGUUUUAGAGC |
| 44 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_20 kb- | downstream | 310 | GUGCUGAGGUCUAACAGAUGGUUUUAGAGC |
| 45 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_20 kb- | downstream | 311 | ACACAACCAGCAAGACUAGCGUUUUAGAGC |
| 46 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_20 kb- | downstream | 312 | GGACACAUUUCUUACCUGACGUUUUAGAGC |
| 47 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_20 kb- | downstream | 313 | GAAGAGCCAGCAUCCUUAGUGUUUUAGAGC |
| 48 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_30 kb- | downstream | 314 | GAGUAUUCUUGUAAGACACG |
| 1 | of CYP2D8 | | |
| TCF20_30 kb- | downstream | 315 | GGUGUAGGGAACCAACACAG |
| 2 | of CYP2D8 | | |
| TCF20_30 kb- | downstream | 316 | UGAUGAGGUGAGCACACACG |
| 3 | of CYP2D8 | | |
| TCF20_30 kb- | downstream | 317 | CUCGGAGUUUUUCACUGGAG |
| 4 | of CYP2D8 | | |
| TCF20_30 kb- | downstream | 318 | UCGUUGUUGUCCUCUACUUU |
| 5 | of CYP2D8 | | |
| TCF20_30 kb- | downstream | 319 | GGCUUUAUCAAAGUGAUCCC |
| 6 | of CYP2D8 | | |
| TCF20_30 kb- | downstream | 320 | AAGCUGAUAUGCAGGAACCC |
| 7 | of CYP2D8 | | |
| TCF20_30 kb- | downstream | 321 | GCAAGUUUUAGGCUAUGUCC |
| 8 | of CYP2D8 | | |
| TCF20_30 kb- | downstream | 322 | GAGCACAACUCUGAGAGGGU |
| 9 | of CYP2D8 | | |
| TCF20_30 kb- | downstream | 323 | AAGUUCUCGGAGUUUUUCAC |
| 10 | of CYP2D8 | | |
| TCF20_30 kb- | downstream | 324 | GAGUAUUCUUGUAAGACACGGUUUAGAGCU |
| 11 | of CYP2D8 | | AUGCU |
| TCF20_30 kb- | downstream | 325 | GGUGUAGGGAACCAACACAGGUUUAGAGCU |
| 12 | of CYP2D8 | | AUGCU |
| TCF20_30 kb- | downstream | 326 | UGAUGAGGUGAGCACACACGGUUUAGAGCU |
| 13 | of CYP2D8 | | AUGCU |
| TCF20_30 kb- | downstream | 327 | CUCGGAGUUUUUCACUGGAGGUUUAGAGCU |
| 14 | of CYP2D8 | | AUGCU |
| TCF20_30 kb- | downstream | 328 | UCGUUGUUGUCCUCUACUUUGUUUAGAGCU |
| 15 | of CYP2D8 | | AUGCU |
| TCF20_30 kb- | downstream | 329 | GGCUUUAUCAAAGUGAUCCCGUUUAGAGCU |
| 16 | of CYP2D8 | | AUGCU |
| TCF20_30 kb- | downstream | 330 | AAGCUGAUAUGCAGGAACCCGUUUAGAGCU |
| 17 | of CYP2D8 | | AUGCU |
| TCF20_30 kb- | downstream | 331 | GCAAGUUUUAGGCUAUGUCCGUUUAGAGCU |
| 18 | of CYP2D8 | | AUGCU |
| TCF20_30 kb- | downstream | 332 | GAGCACAACUCUGAGAGGGUGUUUAGAGCU |
| 19 | of CYP2D8 | | AUGCU |
| TCF20_30 kb- | downstream | 333 | AAGUUCUCGGAGUUUUUCACGUUUAGAGCU |
| 20 | of CYP2D8 | | AUGCU |
| TCF20_30 kb- | downstream | 334 | GAGUAUUCUUGUAAGACACGGUUUUAGAGC |
| 21 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_30 kb- | downstream | 335 | GGUGUAGGGAACCAACACAGGUUUUAGAGC |
| 22 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_30 kb- | downstream | 336 | UGAUGAGGUGAGCACACACGGUUUUAGAGC |
| 23 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_30 kb- | downstream | 337 | CUCGGAGUUUUUCACUGGAGGUUUUAGAGC |
| 24 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_30 kb- | downstream | 338 | UCGUUGUUGUCCUCUACUUUGUUUUAGAGC |
| 25 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_30 kb- | downstream | 339 | GGCUUUAUCAAAGUGAUCCCGUUUUAGAGC |
| 26 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_30 kb- | downstream | 340 | AAGCUGAUAUGCAGGAACCCGUUUUAGAGC |
| 27 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_30 kb- | downstream | 341 | GCAAGUUUUAGGCUAUGUCCGUUUUAGAGC |
| 28 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_30 kb- | downstream | 342 | GAGCACAACUCUGAGAGGGUGUUUUAGAGC |
| 29 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| TCF20_30 kb- | downstream | 343 | AAGUUCUCGGAGUUUUUCACGUUUUAGAGC |
| 30 | of CYP2D8 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA_20 kb- | upstream of | 344 | AACAUUUUCAAUCCGAUGAG |
| 1 | CYP2D6 | | |
| NDUFA_20 kb- | upstream of | 345 | GAAACAUUUUCAAUCCGAUG |
| 2 | CYP2D6 | | |
| NDUFA_20 kb- | upstream of | 346 | AACAUUUUCAAUCCGAUGAGGUUUAGAGCU |
| 3 | CYP2D6 | | AUGCU |
| NDUFA_20 kb- | upstream of | 347 | GAAACAUUUUCAAUCCGAUGGUUUAGAGCU |
| 4 | CYP2D6 | | AUGCU |
| NDUFA_20 kb- | upstream of | 348 | AACAUUUUCAAUCCGAUGAGGUUUUAGAGC |
| 5 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA_20 kb- | upstream of | 349 | GAAACAUUUUCAAUCCGAUGGUUUUAGAGC |
| 6 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA_30 kb- | upstream of | 350 | ACGGACACUACCAAGGAGCG |
| 1 | CYP2D6 | | |
| NDUFA_30 kb- | upstream of | 351 | ACAUGAACGAGGCCAAGCGG |
| 2 | CYP2D6 | | |
| NDUFA_30 kb- | upstream of | 352 | GACACUACCAAGGAGCGCGG |
| 3 | CYP2D6 | | |
| NDUFA_30 kb- | upstream of | 353 | UUUCAGUCGGGACAUGAACG |
| 4 | CYP2D6 | | |
| NDUFA_30 kb- | upstream of | 354 | ACACUACCAAGGAGCGCGGC |
| 5 | CYP2D6 | | |
| NDUFA_30 kb- | upstream of | 355 | UGAGAGGUAGCGGCUUACGU |
| 6 | CYP2D6 | | |
| NDUFA_30 kb- | upstream of | 356 | AAUGGGCUUCACGAAGGUGC |
| 7 | CYP2D6 | | |
| NDUFA_30 kb- | upstream of | 357 | GAAUGUCCCUGUCUACGAUG |
| 8 | CYP2D6 | | |
| NDUFA_30 kb- | upstream of | 358 | CAUGAACGAGGCCAAGCGGA |
| 9 | CYP2D6 | | |
| NDUFA_30 kb- | upstream of | 359 | CGACAGAUGGUGUAGUCCAA |
| 10 | CYP2D6 | | |
| NDUFA_30 kb- | upstream of | 360 | CUUGAAGAACCUCCUCGUGG |
| 11 | CYP2D6 | | |
| NDUFA_30 kb- | upstream of | 361 | GAUGCUUUGGCAAGAUGGCG |
| 12 | CYP2D6 | | |
| NDUFA_30 kb- | upstream of | 362 | UUGAAGAACCUCCUCGUGGC |
| 13 | CYP2D6 | | |
| NDUFA_30 kb- | upstream of | 363 | UCGUGAAGCCCAUUUUCAGU |
| 14 | CYP2D6 | | |
| NDUFA_30 kb- | upstream of | 364 | ACUGAAAAUGGGCUUCACGA |
| 15 | CYP2D6 | | |
| NDUFA_30 kb- | upstream of | 365 | CACGACCCAGCGACCUCCUG |
| 16 | CYP2D6 | | |
| NDUFA_30 kb- | upstream of | 366 | UUCUGAGUGUCUCUCUUCGC |
| 17 | CYP2D6 | | |
| NDUFA_30 kb- | upstream of | 367 | UGACUCUGAGGCUCUCGGAU |
| 18 | CYP2D6 | | |
| NDUFA_30 kb- | upstream of | 368 | AGGUGCCGAACACUGGUGAG |
| 19 | CYP2D6 | | |
| NDUFA_30 kb- | upstream of | 369 | GGACCCCGAGGUAACUGCUG |
| 20 | CYP2D6 | | |
| NDUFA_30 kb- | upstream of | 370 | GGCCUUGAAGAACCUCCUCG |
| 21 | CYP2D6 | | |
| NDUFA_30 kb- | upstream of | 37 | CCCAAGUUGGUGACCUCAGC |
| 22 | CYP2D6 | | |
| NDUFA_30 kb- | upstream of | 372 | CCAGCUGAGGUCACCAACUU |
| 23 | CYP2D6 | | |
| NDUFA_30 kb- | upstream of | 373 | ACGGACACUACCAAGGAGCGGUUUAGAGCU |
| 24 | CYP2D6 | | AUGCU |
| NDUFA_30 kb- | upstream of | 374 | ACAUGAACGAGGCCAAGCGGGUUUAGAGCU |
| 25 | CYP2D6 | | AUGCU |
| NDUFA_30 kb- | upstream of | 375 | GACACUACCAAGGAGCGCGGGUUUAGAGCU |
| 26 | CYP2D6 | | AUGCU |
| NDUFA_30 kb- | upstream of | 376 | UUUCAGUCGGGACAUGAACG |
| 27 | CYP2D6 | | GUUUAGAGCUAUGCU |
| NDUFA_30 kb- | upstream of | 377 | ACACUACCAAGGAGCGCGGC |
| 28 | CYP2D6 | | GUUUAGAGCUAUGCU |
| NDUFA_30 kb- | upstream of | 378 | UGAGAGGUAGCGGCUUACGU |
| 29 | CYP2D6 | | GUUUAGAGCUAUGCU |
| NDUFA_30 kb- | upstream of | 379 | AAUGGGCUUCACGAAGGUGC |
| 30 | CYP2D6 | | GUUUAGAGCUAUGCU |
| NDUFA_30 kb- | upstream of | 380 | GAAUGUCCCUGUCUACGAUG |
| 31 | CYP2D6 | | GUUUAGAGCUAUGCU |
| NDUFA_30 kb- | upstream of | 381 | CAUGAACGAGGCCAAGCGGA |
| 32 | CYP2D6 | | GUUUAGAGCUAUGCU |
| NDUFA_30 kb- | upstream of | 382 | CGACAGAUGGUGUAGUCCAA |
| 33 | CYP2D6 | | GUUUAGAGCUAUGCU |
| NDUFA_30 kb- | upstream of | 383 | CUUGAAGAACCUCCUCGUGG |
| 34 | CYP2D6 | | GUUUAGAGCUAUGCU |
| NDUFA_30 kb- | upstream of | 384 | GAUGCUUUGGCAAGAUGGCG |
| 35 | CYP2D6 | | GUUUAGAGCUAUGCU |
| NDUFA_30 kb- | upstream of | 385 | UUGAAGAACCUCCUCGUGGC |
| 36 | CYP2D6 | | GUUUAGAGCUAUGCU |
| NDUFA_30 kb- | upstream of | 386 | UCGUGAAGCCCAUUUUCAGU |
| 37 | CYP2D6 | | GUUUAGAGCUAUGCU |
| NDUFA_30 kb- | upstream of | 387 | ACUGAAAAUGGGCUUCACGA |
| 38 | CYP2D6 | | GUUUAGAGCUAUGCU |
| NDUFA_30 kb- | upstream of | 388 | CACGACCCAGCGACCUCCUG |
| 39 | CYP2D6 | | GUUUAGAGCUAUGCU |
| NDUFA_30 kb- | upstream of | 389 | UUCUGAGUGUCUCUCUUCGC |
| 40 | CYP2D6 | | GUUUAGAGCUAUGCU |
| NDUFA_30 kb- | upstream of | 390 | UGACUCUGAGGCUCUCGGAU |
| 41 | CYP2D6 | | GUUUAGAGCUAUGCU |
| NDUFA_30 kb- | upstream of | 391 | AGGUGCCGAACACUGGUGAG |
| 42 | CYP2D6 | | GUUUAGAGCUAUGCU |
| NDUFA_30 kb- | upstream of | 392 | GGACCCCGAGGUAACUGCUG |
| 43 | CYP2D6 | | GUUUAGAGCUAUGCU |
| NDUFA_30 kb- | upstream of | 393 | GGCCUUGAAGAACCUCCUCG |
| 44 | CYP2D6 | | GUUUAGAGCUAUGCU |
| NDUFA_30 kb- | upstream of | 394 | CCCAAGUUGGUGACCUCAGC |
| 45 | CYP2D6 | | GUUUAGAGCUAUGCU |
| NDUFA_30 kb- | upstream of | 395 | CCAGCUGAGGUCACCAACUU |
| 46 | CYP2D6 | | GUUUAGAGCUAUGCU |
| NDUFA_30 kb- | upstream of | 396 | ACGGACACUACCAAGGAGCGGUUUUAGAGC |
| 47 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA_30 kb- | upstream of | 397 | ACAUGAACGAGGCCAAGCGGGUUUUAGAGC |
| 48 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA_30 kb- | upstream of | 398 | GACACUACCAAGGAGCGCGGGUUUUAGAGC |
| 49 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA_30 kb- | upstream of | 399 | UUUCAGUCGGGACAUGAACGGUUUUAGAGC |
| 50 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA_30 kb- | upstream of | 400 | ACACUACCAAGGAGCGCGGCGUUUUAGAGC |
| 51 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA_30 kb- | upstream of | 401 | UGAGAGGUAGCGGCUUACGUGUUUUAGAGC |
| 52 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA_30 kb- | upstream of | 402 | AAUGGGCUUCACGAAGGUGCGUUUUAGAGC |
| 53 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA_30 kb- | upstream of | 403 | GAAUGUCCCUGUCUACGAUGGUUUUAGAGC |
| 54 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA_30 kb- | upstream of | 404 | CAUGAACGAGGCCAAGCGGAGUUUUAGAGC |
| 55 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA_30 kb- | upstream of | 405 | CGACAGAUGGUGUAGUCCAAGUUUUAGAGC |
| 56 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA_30 kb- | upstream of | 406 | CUUGAAGAACCUCCUCGUGGGUUUUAGAGC |
| 57 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA_30 kb- | upstream of | 407 | GAUGCUUUGGCAAGAUGGCGGUUUUAGAGC |
| 58 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA_30 kb- | upstream of | 408 | UUGAAGAACCUCCUCGUGGCGUUUUAGAGC |
| 59 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA_30 kb- | upstream of | 409 | UCGUGAAGCCCAUUUUCAGUGUUUUAGAGC |
| 60 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA_30 kb- | upstream of | 410 | ACUGAAAAUGGGCUUCACGAGUUUUAGAGC |
| 61 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA_30 kb- | upstream of | 411 | CACGACCCAGCGACCUCCUGGUUUUAGAGC |
| 62 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA_30 kb- | upstream of | 412 | UUCUGAGUGUCUCUCUUCGCGUUUUAGAGC |
| 63 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA_30 kb- | upstream of | 413 | UGACUCUGAGGCUCUCGGAUGUUUUAGAGC |
| 64 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA_30 kb- | upstream of | 414 | AGGUGCCGAACACUGGUGAGGUUUUAGAGC |
| 65 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA_30 kb- | upstream of | 415 | GGACCCCGAGGUAACUGCUGGUUUUAGAGC |
| 66 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA_30 kb- | upstream of | 416 | GGCCUUGAAGAACCUCCUCGGUUUUAGAGC |
| 67 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA_30 kb- | upstream of | 417 | CCCAAGUUGGUGACCUCAGCGUUUUAGAGC |
| 68 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |
| NDUFA_30 kb- | upstream of | 418 | CCAGCUGAGGUCACCAACUUGUUUUAGAGC |
| 69 | CYP2D6 | | UAGAAAUAGCAAGUUAAAAUAAGGCUAGU |
| | | | CCGUUAUCAACUUGAAAAAGUGGCACCGAG |
| | | | UCGGUGCUUUU |

In various aspects, the methods further comprise identifying one or more genetic variations in CYP2D6. In some cases, the genetic variation is a pharmacogenetically relevant variation in CYP2D6 (e.g., a star allele haplotype). In some cases, the genetic variation is a structural variation in CYP2D6. In some cases, the subject is identified as having a reduction or loss of CYP2D6 function based on the genetic variation. In some cases, the subject is identified as having an increase in or a gain of CYP2D6 function.

In various aspects, the method further comprises recommending a treatment to the subject based on the identifying. In various aspects, the method further comprises treating the subject based on the identifying. In various aspects, the method involves recommending an alternative treatment based on the identifying. In various aspects, the method involves recommending a dosage of a drug based on the identifying. In various aspects, the method involves altering a dosage (or recommending the alteration of a dosage) of a drug (e.g., that is activated by or metabolized by CYP2D6) administered to the subject. In some cases, the drug (or therapeutic) is a drug that is activated or metabolized by CYP2D6.

### Compositions and Kits

In one aspect, provided herein are compositions and kits comprising: (a) a Clustered Regularly Interspaced Short Palindromic Repeat (CRISPR)-associated endonuclease; (b) an outer pair of gRNAs comprising: (i) a first outer gRNA comprising a nucleotide sequence that is substantially complementary to a first nucleotide sequence present in genomic DNA that is upstream of a genomic region of interest; and (ii) a second outer gRNA comprising a nucleotide sequence that is substantially complementary to a second nucleotide sequence present in genomic DNA that is downstream of said genomic region of interest; (c) an inner pair of gRNAs comprising: (iii) a first inner gRNA comprising a nucleotide sequence that is substantially complementary to a third nucleotide sequence present in genomic DNA that is upstream of said genomic region of interest; and (iv) a second inner gRNA comprising a nucleotide sequence that is substantially complementary to a fourth nucleotide sequence present in genomic DNA that is downstream of said genomic region of interest, wherein the third nucleotide sequence and the fourth nucleotide sequence are present on the genomic DNA at a base length closer to the genomic region of interest than the first nucleotide sequence and the second nucleotide sequence.

In some cases, the compositions and/or kits further include an exonuclease. The exonuclease may be selected from the group consisting of: exonuclease I, exonuclease II, exonuclease III, exonuclease IV, exonuclease V, exonuclease VI, exonuclease VII, and exonuclease VIII.

The CRISPR-associated endonuclease can be any CRISPR-associated endonuclease described herein. In some cases, the CRISPR-associated endonuclease is a Class I or a Class II CRISPR-associated endonuclease. Non-limiting examples of Cas I CRISPR-associated endonucleases include, Non-limiting examples of Class II CRISPR-associated endonucleases include, Cas3, Cas5, Cas8a, Cas8b, Cas8c, Cas10d, Cse1, Cse2, Csy1, Csy2, Csy3, GSU0054, Cas10, Csm2, Cmr5, Csx11, Csx10, and Csf1. Cas9, Cas12a, Csn2, Cas4, Cas12b, Cas12c, Cas13a, Cas13b, Cas13c, and Cas13d. In some cases, the CRISPR-associated endonuclease is a Cas protein or polypeptide. In some embodiments, the CRISPR-associated endonuclease is a Cas12a protein or polypeptide.

In some embodiments, the CRISPR-associated endonuclease is a Cas9 protein or polypeptide. In some cases, the Cas9 protein or polypeptide is derived from the bacterial species Streptococcus pyogenes. In some cases, the Cas9 protein or polypeptide has an amino acid sequence identical to a wild-type Cas9 amino acid sequence. In other cases, the Cas9 protein or polypeptide has an amino acid sequence that is modified relative to a wild-type Cas9 amino acid sequence. In some cases, the Cas9 protein or polypeptide has one or more mutations (e.g., relative to a wild-type Cas9 protein or polypeptide). In some cases, the one or more mutations is a substitution, a deletion, or an insertion. The Cas9 protein or polypeptide may have an amino acid sequence having at least about 50% sequence identity relative to a wild-type Cas9 protein or polypeptide. For example, the Cas9 protein or polypeptide may have at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity relative to a wild-type Cas9 protein or polypeptide. In some cases, the Cas9 variant may comprise one or more point mutations relative to a wild-type S. pyogenes Cas9. For example, the Cas9 variant may comprise a point mutation relative to a wild-type S. pyogenes Cas9 selected from the group consisting of: R780A, K810A, K848A, K855A, H982A, K1003A, R1060A, D1135E, N497A, R661A, Q695A, Q926A, L169A, Y450A, M495A, M694A, and M698A.

In some cases, the genomic region of interest is a genetic locus comprising CYP2D6, CYP2D7, and CYP2D8. In some cases, at least one of the gRNAs (e.g., at least one of the first inner gRNA, the second inner gRNA, the first outer gRNA, and the second outer gRNA) comprises a nucleotide sequence according to any nucleotide sequence provided in Table 1 (e.g., SEQ ID NOs: 1-418). In some cases, at least one of the gRNAs (e.g., at least one of the first inner gRNA, the second inner gRNA, the first outer gRNA, and the second outer gRNA) comprises a nucleotide sequence having at least about 90% (e.g., at least about 91%, at least about 92%, at least about 93%, at least about 94%, at least about 95%, at least about 96%, at least about 97%, at least about 98%, at least about 99%) sequence identity to any nucleotide sequence provided in Table 1 (e.g., SEQ ID NOs: 1-418). In some cases, at least one of the gRNAs is a crRNA. In some cases, at least one of the gRNAs is an sgRNA. In some cases, the first outer guide RNA, the first inner guide RNA, or both, comprise the nucleotide sequence of any one of SEQ ID NOS: 3-12, 17-26, 68-77, 82-214, and 344-418. In some cases, the second outer guide RNA, the second inner guide RNA, or both, comprise the nucleotide sequence of any one of SEQ ID NOS: 1, 2, 13-16, 27-67, 78-81, and 215-343.

In some aspects, the kit further comprises instructions for using the kit in any method provided herein. In some cases, the kit further comprises instructions for using the kit in a nested CRISPR reaction (e.g., as described herein). In some cases, the kit further comprises instructions for using the kit in a method to excise the genomic region of interest from genomic DNA (e.g., as described herein). In some cases, the kit further comprises instructions for using the kit in a method to excise the CYP2D6 locus from genomic DNA (e.g., as described herein).

### Subjects & Biological Samples

A subject can provide a biological sample for genetic analysis. The biological sample can be any substance that is produced by the subject. Generally, the biological sample is any tissue taken from the subject or any substance produced by the subject. The biological may be a body fluid, such as, blood (e.g., whole blood, plasma, serum), urine, saliva, bone marrow, spinal fluid, sputum, ascites, lymphatic fluid, pleural fluid, amniotic fluid, semen, vaginal fluid, sweat, stool, glandular secretions, ocular fluids, breast milk, and the like. The biological sample may be a cells and/or a solid tissue (e.g., cheek tissue (e.g., from a cheek swab), feces, skin, hair, organ tissue, and the like). In some cases, the biological sample is a solid tumor or a biopsy of a solid tumor. In some cases, the biological sample is a formalin-fixed, paraffin-embedded (FFPE) tissue sample. The biological sample can be any biological sample that comprises genomic DNA.

Biological samples may be derived from a subject. The subject may be a mammal, a reptile, an amphibian, an avian, or a fish. The mammal may be a human, ape, orangutan, monkey, chimpanzee, cow, pig, horse, rodent, bird, reptile, dog, cat, or other animal. A reptile may be a lizard, snake, alligator, turtle, crocodile, and tortoise. An amphibian may be a toad, frog, newt, and salamander. Examples of avians include, but are not limited to, ducks, geese, penguins, ostriches, and owls. Examples of fish include, but are not limited to, catfish, eels, sharks, and swordfish. Preferably, the subject is a human. The subject may have a disease or condition. The subject may be prescribed a therapeutic. The therapeutic may be a therapeutic that is activated by and/or metabolized by CYP2D6.

### Systems of the Disclosure

Further provided herein are systems for performing the methods provided herein. In one aspect, a system is provided comprising (a) at least one memory location configured to receive a data input comprising data generated from any method described herein; and (b) a computer processor operably coupled to the at least one memory location, wherein the computer processor is programmed to generate an output based on the data.

In various aspects, the output is a report. In various aspects, the output is a genotype of the complex genomic region of interest. In various aspects, the output is a genetic sequence of the complex genomic region of interest. In various aspects, the output is a structural analysis of the complex genomic region of interest. In various aspects, the analyzing comprises genotyping the complex genomic region of interest. In various aspects, the analyzing comprises performing structural analysis of the complex genomic region of interest. In various aspects, the analyzing comprises sequencing the complex genomic region of interest.

In various aspects, the output identifies genetic variation in CYP2D6. In various aspects, the output identifies a decrease in, a loss of, or an increase in a function of CYP2D6. In various aspects, the report recommends a treatment to the subject based on the genetic variation. In various aspects, the report recommends a dosage of a therapeutic to the subject based on the genetic variation. In various aspects, the report recommends altering a dosage of a therapeutic based on the genetic variation. In some cases, the therapeutic is a therapeutic that is activated by or metabolized by CYP2D6.

The disclosure further provides computer-based systems for performing the methods described herein. In some aspects, the systems can be used for analyzing data generated by a method provided herein. The system can comprise one or more client components. The one or more client components can comprise a user interface. The system can comprise one or more server components. The server components can comprise one or more memory locations. The one or more memory locations can be configured to receive a data input. The data input can comprise sequencing data. The sequencing data can be generated from a nucleic acid sample (e.g., genomic DNA) from a subject. Non-limiting examples of sequencing data suitable for use with the systems of this disclosure have been described. The system can further comprise one or more computer processor. The one or more computer processor can be operably coupled to the one or more memory locations. The one or more computer processor can be programmed to generate an output for display on a screen. The output can comprise one or more reports.

The systems described herein can comprise one or more client components. The one or more client components can comprise one or more software components, one or more hardware components, or a combination thereof. The one or more client components can access one or more services through one or more server components. The one or more services can be accessed by the one or more client components through a network. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing. The network, in some cases with the aid of the computer system, can implement a peer-to-peer network, which may enable devices coupled to the computer system to behave as a client or a server.

The systems can comprise one or more memory locations (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. The memory, storage unit, interface and peripheral devices are in communication with the CPU through a communication bus, such as a motherboard. The storage unit can be a data storage unit (or data repository) for storing data. In one example, the one or more memory locations can store the received sequencing data.

The systems can comprise one or more computer processors. The one or more computer processors may be operably coupled to the one or more memory locations to e.g., access the stored data. The one or more computer processors can implement machine executable code to carry out the methods described herein.

The machine executable or machine readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processer adapted to execute the code, can be compiled during runtime, or can be interpreted during runtime. The code can be supplied in a programming language that can be selected to enable the code to execute in a pre-compiled, as-compiled or interpreted fashion.

Aspects of the systems and methods provided herein, such as the computer system, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The systems disclosed herein can include or be in communication with one or more electronic displays. The electronic display can be part of the computer system, or coupled to the computer system directly or through the network. The computer system can include a user interface (UI) for providing various features and functionalities disclosed herein. Examples of UIs include, without limitation, graphical user interfaces (GUIs) and web-based user interfaces. The UI can provide an interactive tool by which a user can utilize the methods and systems described herein. By way of example, a UI as envisioned herein can be a web-based tool by which a healthcare practitioner can order a genetic test, customize a list of genetic variants to be tested, and receive and view a report.

The methods disclosed herein may comprise biomedical databases, genomic databases, biomedical reports, disease reports, case-control analysis, and rare variant discovery analysis based on data and/or information from one or more databases, one or more assays, one or more data or results, one or more outputs based on or derived from one or more assays, one or more outputs based on or derived from one or more data or results, or a combination thereof.

As described herein, one or more computer processors can implement machine executable code to perform the methods of the disclosure. Machine executable code can comprise any number of open-source or closed-source software. The machine executable code can be implemented to analyze a data input. The data input can be sequencing data generated from one or more sequencing reactions. The computer process can be operably coupled to at least one memory location. The computer processor can access the data (e.g., sequencing data) from the at least one memory location. In some cases, the computer processor can implement machine executable code to map the sequencing data to a reference sequence. In some cases, the computer processor can implement machine executable code to determine a presence or absence of a genetic variant from the sequencing data. In some cases, the computer processor can implement machine executable code to generate an output for display on a screen (e.g., a report).

Machine executable code may comprise one or more algorithms. The one or more algorithms may be used to implement the methods of the disclosure.

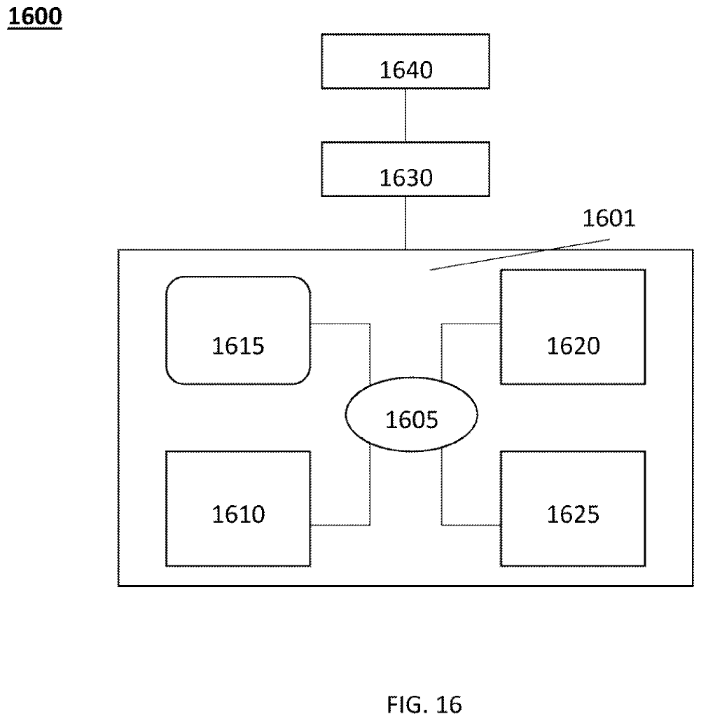

The systems of the disclosure may comprise one or more computer systems. FIG. 16 shows a computer system (also “system” herein) 1601 programmed or otherwise configured to implement the methods of the disclosure, such as receiving data and producing an output based on said data. The system 1601 includes a central processing unit (CPU, also “processor” and “computer processor” herein) 1605, which can be a single core or multi core processor, or a plurality of processors for parallel processing. The system 1601 also includes memory 1610 (e.g., random-access memory, read-only memory, flash memory), electronic storage unit 1615 (e.g., hard disk), communications interface 1620 (e.g., network adapter) for communicating with one or more other systems, and peripheral devices 1625, such as cache, other memory, data storage and/or electronic display adapters. The memory 1610, storage unit 1615, interface 1620 and peripheral devices 1625 are in communication with the CPU 1605 through a communications bus (solid lines), such as a motherboard. The storage unit 1615 can be a data storage unit (or data repository) for storing data. The system 1601 is operatively coupled to a computer network (“network”) 1630 with the aid of the communications interface 1620. The network 1630 can be the Internet, an internet and/or extranet, or an intranet and/or extranet that is in communication with the Internet. The network 1630 in some cases is a telecommunication and/or data network. The network 1630 can include one or more computer servers, which can enable distributed computing, such as cloud computing. The network 1630 in some cases, with the aid of the system 1601, can implement a peer-to-peer network, which may enable devices coupled to the system 1601 to behave as a client or a server.

The system 1601 is in communication with a processing system 1640. The processing system 1640 can be configured to implement the methods disclosed herein, such as mapping sequencing data to a reference sequence or assigning a classification to a genetic variant. The processing system 1640 can be in communication with the system 1601 through the network 1630, or by direct (e.g., wired, wireless) connection. The processing system 1640 can be configured for analysis, such as nucleic acid sequence analysis.

Methods and systems as described herein can be implemented by way of machine (or computer processor) executable code (or software) stored on an electronic storage location of the system 1601, such as, for example, on the memory 1610 or electronic storage unit 1615. During use, the code can be executed by the processor 1605. In some examples, the code can be retrieved from the storage unit 1615 and stored on the memory 1610 for ready access by the processor 1605. In some situations, the electronic storage unit 1615 can be precluded, and machine-executable instructions are stored on memory 1610.

The code can be pre-compiled and configured for use with a machine having a processer adapted to execute the code, can be compiled during runtime or can be interpreted during runtime. The code can be supplied in a programming language that can be selected to enable the code to execute in a pre-compiled, as-compiled or interpreted fashion.

Aspects of the systems and methods provided herein can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1601 can include or be in communication with an electronic display that comprises a user interface (UI). Examples of UI's include, without limitation, a graphical user interface (GUI) and web-based user interface.

In some embodiments, the system 1601 includes a display to provide visual information to a user. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In still further embodiments, the display is a combination of devices such as those disclosed herein. The display may provide one or more biomedical reports to an end-user as generated by the methods described herein.

In some embodiments, the system 1601 includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera to capture motion or visual input. In still further embodiments, the input device is a combination of devices such as those disclosed herein.

The system 1601 can include or be operably coupled to one or more databases. The databases may comprise genomic, proteomic, pharmacogenomic, biomedical, and scientific databases. The databases may be publicly available databases. Alternatively, or additionally, the databases may comprise proprietary databases. The databases may be commercially available databases. The databases include, but are not limited to, MendelDB, PharmGKB, Varimed, Regulome, curated BreakSeq junctions, Online Mendelian Inheritance in Man (OMIM), Human Genome Mutation Database (HGMD), NCBI dbSNP, NCBI RefSeq, GENCODE, GO (gene ontology), and Kyoto Encyclopedia of Genes and Genomes (KEGG).

Data can be produced and/or transmitted in a geographic location that comprises the same country as the user of the data. Data can be, for example, produced and/or transmitted from a geographic location in one country and a user of the data can be present in a different country. In some cases, the data accessed by a system of the disclosure can be transmitted from one of a plurality of geographic locations to a user. Data can be transmitted back and forth among a plurality of geographic locations, for example, by a network, a secure network, an insecure network, an internet, or an intranet.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the embodiments of the disclosure. Changes therein and other uses which are encompassed within the spirit of the disclosure as defined by the scope of the claims will occur to those skilled in the art.

### Example 1

### CYP2D6 and Clinical Testing

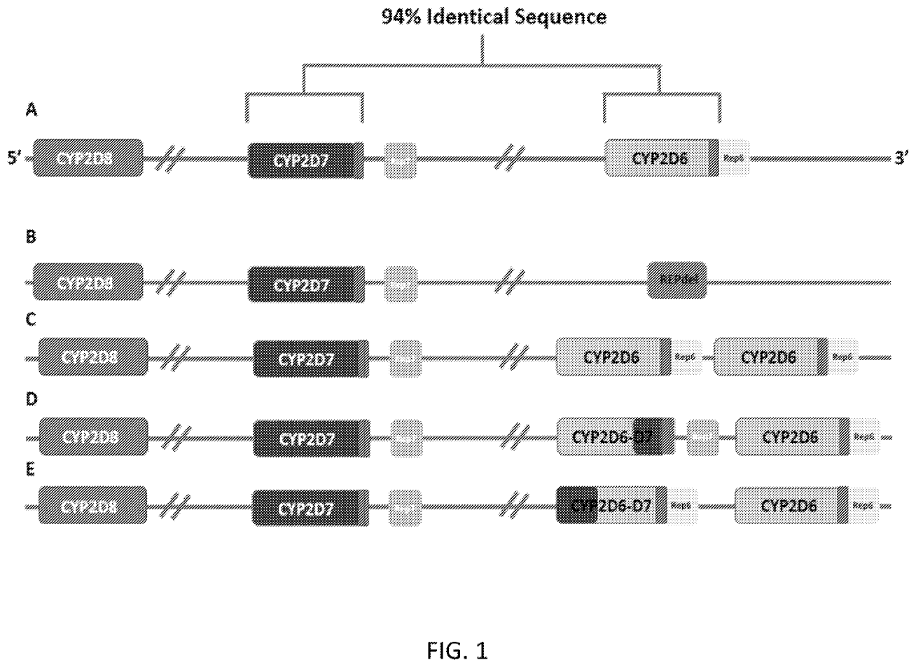

CYP2D6 Genetic Structure: CYP2D6 is a small gene (4382 bp) and has nine exons. However, genetic analysis of this highly polymorphic gene locus is difficult due to the presence of the highly similar nonfunctional CYP2D7 and CYP2D8 pseudogenes within the locus, as shown in FIG. 1. The similarity between CYP2D6 and CYP2D7 and the presence of large repeat regions has generated not only gene deletions and gene duplications, but also complex gene hybrids that contain either 3′ CYP2D7 with 5′ CYP2D6 or 3′ CYP2D6 and 5′ CYP2D7. Currently, multiple testing assays are required to detect the presence of these structural variations.

Current Platforms for Testing: One common method to analyze CYP2D6 is by sequence analysis of long-range, allele-specific PCR products. Briefly, allele-specific primers are employed to amplify targeted regions. Single-nucleotide variants (SNVs) found on the PCR product represent that allele's haplotype. Allele-specific amplicons can also be generated from duplicated gene copies and CYP2D6-2D7 and CYP2D7-2D6 hybrid genes. More recently, long-read sequencing technologies such as single molecule real-time (SMRT) sequencing or Nanopore sequencing have also been used to more accurately characterize CYP2D6 haplotypes; however, limitations remain with library generation for long-read CYP2D6 sequencing. XL-PCR reactions currently used to generate CYP2D6 templates for sequencing are limited by the size of product that can be generated, are primer-specific, and do not capture complex hybrids or many known CNVs unless the variation was previously characterized and is known to be present in the sample of interest.

In summary, CYP2D6 is a highly polymorphic gene that is directly involved in the metabolism of ˜25% of all prescribed drugs. Genetic variation in the gene, including copy number changes can directly impact the drug metabolizing status of a patient. An accurate genotype that includes copy number is critical and current methodologies cannot fully assay the complexity of the gene region.

Proposed herein is a method to utilize CRISPR/Cas9 technology and site-specific adapter ligation in combination with long-read sequencing to develop a diagnostic quality methodology for CYP2D6 analysis. The approach utilizes a single sample-agnostic CRISPR cleavage step to isolate the entire CYP2D6 locus for long-read sequencing. This methodology is able to accurately detect both single nucleotide polymorphisms (SNPs) and CNVs, and assign the most accurate, phased CYP2D6 genotype and metabolizer status possible.

CRISPR technology can be used to target and excise genomic regions of interest (ROI), both in vitro and in vivo. Briefly, the CRISPR-C-associated protein 9 (Cas9), when complexed with synthetically generated target-specific guide RNA (sgRNA), creates a double-stranded cut at a sequence with complementarity to the target-specific sequence of the guide RNA. By designing sgRNAs to target sequences at both ends of an ROI, CRISPR-Cas9 can be used to excise the DNA, which can be up to megabases in length.

Long-read sequencing: While the development of short-read next-generation sequencing (NGS) has revolutionized human genetics, the limitations are well recognized. Long-read sequencing of isolated HMW DNA fragments has recently sparked interest as it allows one to obtain phasing information, identify small structural variation and better assemble high-complexity regions of the genome, including tandem repeats. The use of CRISPR technology to isolate DNA fragments in a target-specific manner offers an innovative and elegant approach to target relevant regions of the genome for long-read sequencing.

The GeT-RM Cohort: As part of a major effort to systematically characterize the CYP2D6 gene structure, CYP2D6 genotyping data has been provided to establish a state-of-the-art set of well-characterized reference material for assay development, validation, quality control and proficiency testing. This effort was conducted in collaboration with the Genetic Testing Reference Materials Coordination Program (GeT-RM) at the Centers for Disease Control and Prevention-based Genetic Testing Reference Material Coordination Program, the Coriell Institute for Medical Research, as well other PGx community members. As part of this study, Pharmacoscan™ based CYP2D6 genotyping was provided on several samples that contained complex structural arrangements and/or rare CYP2D6 genotypes. This data, in conjunction with XL-PCR based NGS analysis was used to determine the most accurate genotype of these samples possible with current analysis methodologies. The information on all cell lines and consensus genotyping and annotation data builds the foundation for the validation of the proposed new sequencing and analysis approach.

### Research Design and Methods

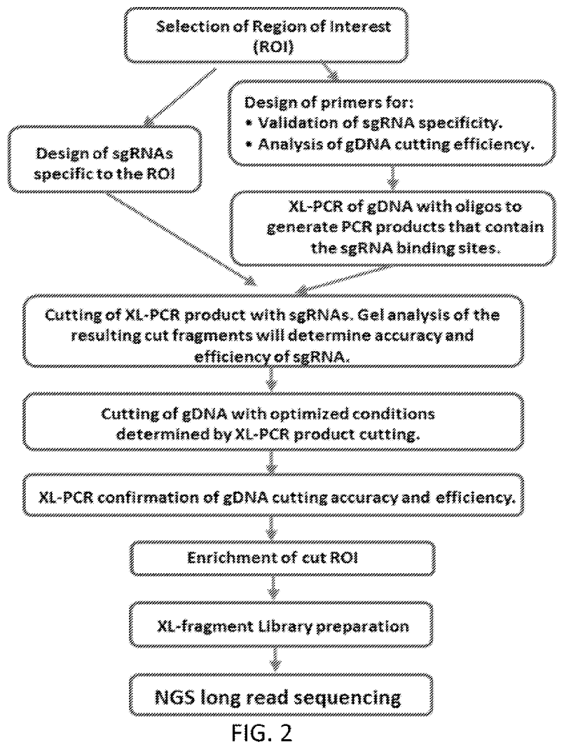

Aim 1 (Method Development): (a) Optimization of a specific CRISPR/Cas9 methodology for creation of high-molecular weight DNA segments containing the CYP2D6-D7 genomic loci for subsequent size analysis (e.g., gel) in genomic human DNA (e.g., blood sample). (b) Isolation/enrichment of targeted region and generation of XL-libraries for sequencing. (c) Establishment of NGS approach for long template sequencing of genomic variants in CYP2D6-D7 genomic loci (e.g., PacBio, MinION). An outline of the proposed workflow is depicted in FIG. 2.

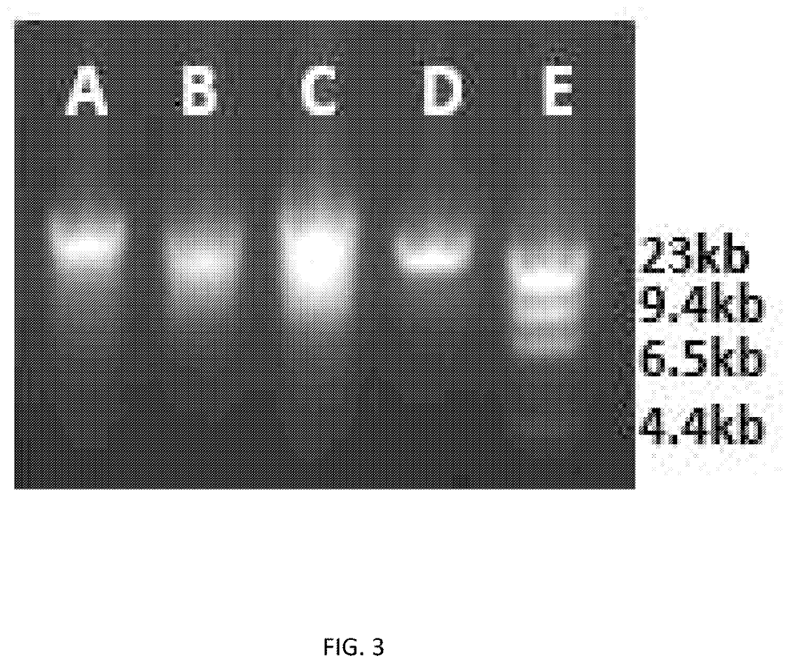

Isolation of HMW DNA: The normal length of ROI (CYP2D6 and CYP2D7) is 28-35 kb. To ensure the entire ROI is intact for downstream analysis, a protocol was developed using the NucleoBond® Genomic DNA and RNA purification system to isolate high molecular weight gDNA (up to 70 kb). The modified protocol enables the extraction of gDNA with molecular weight >50 kb, compared to 10 kb-50 kb range observed with other methodologies (FIG. 3).

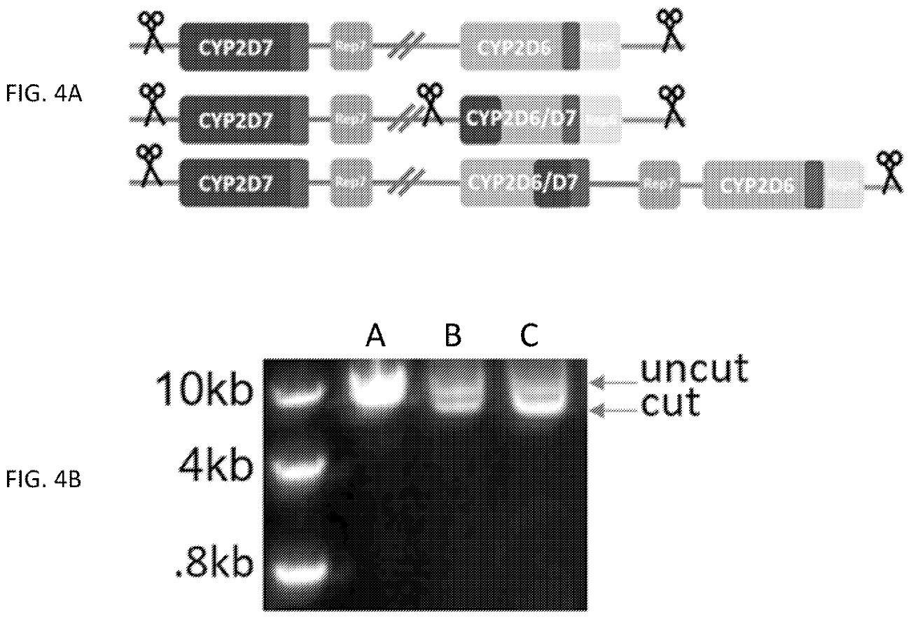

Design and validation of highly specific sgRNAs: Due to the complex and highly polymorphic nature of the CYP2D6 loci, traditional PCR and array-based technologies require multiple assays to perform both CNV and SNP analysis. CRISPR Cas9 approaches that target only the CYP2D6 gene fail to capture alleles that contain a structural variation, such as a D6/D7 hybrid allele or CYP2D6 duplication event. To overcome this limitation, unique sequences were identified that flank the region encompassing both CYP2D6 and CYP2D7. By designing the sgRNAs to target these unique regions, one CRISPR/Cas9 cleavage reaction was performed to isolate the entire CYP2D6/CYP2D7 region (FIG. 4A).

To confirm the specificity and efficacy of the sgRNAs, XL-PCR products that contain the targeted sgRNA binding sites were generated from gDNA. The XL-PCR products were incubated with either Cas9 and no sgRNA (FIG. 4B, sample A) or Cas9 and different sgRNAs (FIG. 4B, samples B and C). All PCR products incubated with Cas9 and sgRNA were cleaved to produce DNA fragments of the expected size but different sgRNAs showed different degrees of cleavage efficiency.

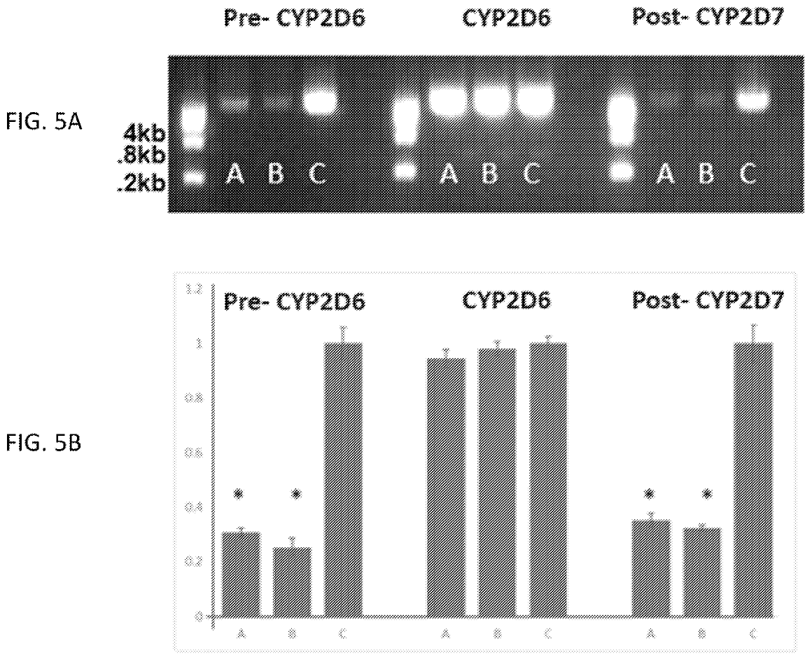

Cutting of CYP2D6-CYP2D7 loci in genomic DNA: The sgRNAs must bind with high efficiency and specificity to gDNA, which may contain off-target recognition sites. To interrogate the CRISPR cutting efficiency and specificity, genomic DNA was incubated with either Cas9 and no sgRNA (negative control) or Cas9 and a pool of two sgRNAs that cut 5′ of CYP2D6 and 3′ of CYP2D7. PCR reactions were performed with primers flanking each predicted cleavage site. If the sgRNAs bind to the correct binding sites and cleavage occurs, one would expect a reduction in PCR product. Indeed, this is what is observed (FIG. 5A, FIG. 5B). PCR was also performed on the CYP2D6 locus using primers internal to the sgRNA binding sites to determine whether Cas9-mediated off-target cleavage occurred within the CYP2D6 gene. No evidence of off-target cleavage within CYP2D6 was observed (FIG. 5A, FIG. 5B).

In summary, it was demonstrated by XL-PCR and genomic DNA interrogation that the Cas9-sgRNA complex cuts on both sides of the targeted CYP2D6-CYP2D7 locus with high efficiency and without significant off-target activity within the locus. Cleavage creates a predicted 28 kb fragment, which can be utilized for down-stream long-read NGS after enrichment.

### Example 2. Further Optimization of CRISPR/Cas9 Methodology

Other sgRNA and Cas enzymes are developed and tested. Standard software is used to identify and design sgRNAs that are tested as described above. The goal is to obtain sgRNA that cleave at the ROI with high efficiency and specificity. Preference is given to shorter DNA fragments, which still contain the full ROI. Shorter fragments might have the benefit of reduced sequencing and processing cost. Cleavage of the same region with the CRISPR Cas12a enzyme is also attempted. The Cas12a endonuclease functions similarly to Cas9 but has a different PAM sequence requirement (TTTV) and produces a 5′ staggered overhang after cleavage. In contrast, Cas9 produces blunt ends. This has importance for the subsequent step.

### Example 3. Enrichment of CYP2D6-CYP2D7 Loci in Genomic DNA

As a proof of concept, 5 μg of gDNA was cut with Cas9-sgRNA targeting cleavage sites 5′ of CYP2D6 and 3′ of CYP2D7 as described above. The cleaved DNA was run on the BluePippen (Sage Science) instrument using a 0.75% agarose gel cassette, which allows for size selection in the range of 1-50 kb. The eluted sample was confirmed to contain the desired CYP2D6-CYP2D7 locus using PCR. While this gel-based approach allows for the isolation of HMW samples, there are several drawbacks, including time (˜10-12 hours per Blue Pippen run), limited sample number (4-5 samples per run), significant loss of material/poor recovery and high cost per sample (˜$50.00).

To overcome these limitations, several approaches to target enrichment are tested. This allows the identification of pros and cons of the various methods and to ultimately identify the most suitable approach for further clinical test development. This is a typical approach to clinical diagnostic test development. The discussion of long-read sequencing below refers to Oxford Nanopore (ONT) sequencing; however, any of the protocols can be adapted with few modifications to fit PacBio sequencing requirements.

### Method 1: Amplification-Free Enrichment of Target

DNA preparation: This amplification-free library preparation method involves dephosphorylation of the DNA sample and 3′-end capping, followed by CRISPR treatment and site-specific ONT adapter ligation. In the first step, the gDNA is treated with Shrimp Alkaline Phosphatase, which removes phosphate groups from the 5′ ends of DNA fragments, and Terminal Transferase which adds a single thymidine dideoxynucleotide to the 3′ ends. This step ensures that the gDNA ends are incapable of ligation. The DNA is then treated with CRISPR Cas9:gRNA complexes, resulting in blunt-ended ˜28-35 kb CYP2D6/CYP2D7 fragments (see previous paragraphs for details). This is followed by an “A-tailing” step, in which adenosine nucleotides are added to the free 3′ ends of the DNA (e.g., the ends not capped with a ddTTP) with a DNA polymerase. Finally, ONT adapters with thymidine overhangs are added to the DNA. Only the DNA ends produced by CRISPR-Cas9 cleavage ligate to the adapters because they are the only ends with a complementary 3′-overhang and a 5′-phosphate group.

Sequencing: The resulting library is sequenced directly on an ONT instrument. If the quantity of DNA library generated by this method proves challenging for ONT sequencing, this may be overcome by multiplexing samples prior to sequencing and/or by increasing the input gDNA quantity. Furthermore, the background can be reduced by treating the sample with exonucleases (ONT adapters are resistant to Exonuclease III and Lambda Exonuclease), which result in the degradation of all background DNA.

### Method 2: Enrichment Using In Vitro Transcription

Rationale: If the previous approach fails to generate sufficient DNA or if there is an excess of background DNA, an alternative approach is evaluated of targeted amplification via in vitro transcription (IVT). IVT has a few advantages over PCR. (1) Transcription is less likely to propagate errors. (2) Transcription can produce RNA molecules as long as 20-30 kb in length, longer than the size of most long-range PCR products.

DNA preparation: After CRISPR cleavage, DNA is treated with an exonuclease to generate staggered ends, and double-stranded DNA fragments containing a T7 promoter and an overhang complementary to the staggered ends of the CYP26-CYP2D7 locus is ligated to the target fragment. A DNA polymerase and DNA ligase is used to fill in the gaps and seal any nicks. Phage T7 RNA polymerase is able to produce transcripts as long as ˜20 kb. Since promoters are ligated to both ends of the ˜28 kb locus, the longest transcripts produced by T7 RNA polymerase from the promoters at the ends of the locus may be sufficiently long to cover the entire region. However, a large percentage of T7 products are typically less than 4 kb in length. The recently discovered Syn5 cyanophage RNA polymerase is capable of producing transcripts as long as 30 kb. The Syn5 promoter is tested alongside the T7 promoter.

In vitro transcription: IVT is performed with the T7 and Syn5 RNA polymerases. The former enzyme is commercially available while the latter enzyme has been expressed and purified in our laboratory. There are several commercial T7 RNA polymerase IVT kits that are optimized to produce long RNA transcripts. Previous work has shown that T7 promoter sequences randomly inserted in the human genome produce a significant fraction of RNA transcripts larger than 5 kb during IVT. Total RNA yield, the proportion of large transcripts (>15 kb) and error rates are key factors in determining which polymerase and IVT method are superior options. Because a wide range of RNA transcript lengths are likely to be produced, SPRI beads may be used to select the largest transcripts. The RNA is sequenced directly on an ONT instrument.

### Method 3: Multi-Site Introduction of Promoter for In Vitro Transcription

Rationale: If the above approach is insufficient, T7 or Syn5 promoters are inserted at multiple sites across the targeted region. A potential problem with this approach is that fragmentation of the locus makes it challenging to unambiguously assign variants to CYP2D7 or CYP2D6 (because the gene and pseudogene share ˜94% sequence identity) and to derive phasing information. To overcome this limitation, multiple staggered insertion sites are used to generate overlapping fragments.

Introduction of promoter: CRISPR cleavage takes place at ROI flanking sites and at regularly spaced (˜10 kb) apart sites within the locus. Cleavages are made in two separate reactions, each with a different set of target sites, so that the resulting overlapping fragments can be used to stitch reads together after sequencing. Exonuclease treatment, ligation of promoter-containing adapters, IVT, and cDNA synthesis are described above. Promoter-containing adapters contain a short fixed sequence immediately downstream of the promoter. A primer with complementarity to this fixed sequence is used for reverse transcription (RT) when cDNA synthesis is performed. If the RNA produced by IVT spans the length between two insertion sites, a RT primer specific to this sequence selects for cDNA molecules that span the same region.

Potential alternatives: If necessary, a few cycles of long-range PCR, using the fixed sequence at the beginning of each IVT product, may be used to selectively amplify cDNA molecules that span insertion sites.

Potential alternatives: RNA sequencing by ONT requires a large amount of RNA. If necessary, cDNA synthesis is performed with primers that anneal to sites far (15-20 kb) from the start of transcription to select for long transcripts. If a significant proportion of sequencing reads do not map to the target locus, it will be attempted to prevent the ligation of adapters to non-target sites. Dephosphorylation of gDNA before CRISPR treatment and capping the ends of the gDNA with so-called “dumbbell” adapters are two possible options.

### Example 4. Establishment of NGS Approach to Long Template Sequencing of Variants

Methods: Currently there are two major commercial platforms that are amenable to the development of potential diagnostic tests. PacBio has been the first and most prominent technology for long-read sequencing, but associated costs are significant. More recently, nanopore sequencing technology has emerged as a cost effective and potentially feasible platform. Oxford Nanopore (ONT) as a platform continues to mature with regard to through-put, cost and accuracy. Here, ONT is focused on, given these advantages. Nevertheless, the proposed methodologies and methods are, in large part, platform-agnostic and can be modified to fit any of the two current or future long-read platforms. Sequencing runs are performed on the Oxford Nanopore MinION.

Aim 2 (Validation): (a) Perform sequence analysis using current software and platforms for long-read sequence alignment to perform variant calling, CNV analysis and phasing. (b) Compare CYP2D6-D7 long-read sequence analysis results with sequence/copy number variation and characterize consensus genotyping and annotation results with those from the Get-RM project to estimate performance characteristics and guidance towards further diagnostic test development. The feasibility of each method is tested and compared with respect to time- and cost-effectiveness, minimization of required steps and quality of results. The overarching goal is the selection of the most suitable method for isolating, enriching, and sequencing of the entire CYP2D6 gene.

Choice of samples for validation: Once a sample preparation method is developed, an expanded set of additional samples with known genotypes and haplotypes will be analyzed. Samples with complex structure such as duplications, hybrids, selected deletions, and complex rearrangements are included in order to evaluate the platform on an expanded dataset. The samples are selected from the GeT-RM project (see above, “The GeT-RM Cohort”). These cell lines and data provide a unique resource as they allow the evaluation of the novel long-read sequence data against the current gold standard. For this proposal, a subset of these cell lines has been acquired—LCL cell lines. Additional samples for the characterization of other relevant variants and haplotypes from cell line repositories and through existing collaborations are obtained. To further validate the methodology with additional samples, additional cell lines are utilized from the NIST Coriell cohort, which is extensively characterized, including whole genome sequencing. In addition, additional sample types representative of typical diagnostic specimens are acquired, including whole blood and saliva. In total, 48 cell lines are selected for sequencing in this aim, representing duplications, deletions, hybrids and tandem arrangements. The analysis is conducted in duplicate for a total of 96 sequenced samples.

Variant Calling, CNV Calling, and Phasing: Software packages specifically developed for long-read ONT data are used. Clair is a recent update to the Clairvoyante, a multi-task five-layer convolutional neural network model for predicting variant type, zygosity, alternative allele and Insertion/deletion length. An additional package, which has recently been developed, is Megalodon. Megalodon's functionality centers on the anchoring of high-information neural network base-calling to a reference sequence. The performance characteristics of the Nanopore technology have recently been evaluated by Bowden et al. for whole genome sequencing using a standard reference sample. The consensus accuracy at 82× coverage was 99.9%, although the data also shows some current limitations of the platform. As the proposal is to sequence only a small targeted region, and given the ability to sequence the region at ultra-high depth, it is expected that the current analysis platforms produce sufficiently accurate data of the targeted sequence. Future software developments are also monitored and new methods are utilized as they become available.

Comparison to consensus data: The data is compared with the GeT-RM consensus results (which are based on the results from all the platforms, as well as an expert panel review of variants). The concordance for haplotype-calling SNPs and CNVs is determined, the ability to identify sequence features of hybrid haplotypes is evaluated, and concordance to determine metabolizer status is measured. Next, the additional variants are compared with genotyping data from the GeT-RM project. The data is analyzed in conjunction with phasing information (e.g., the determined haplotypes) to determine whether the phased genotyping data is consistent with the results, as this provides non-imputed phasing information. Finally, any additional variants identified through sequencing alone are identified. An exploratory sequence comparison between CYP2D6 and its pseudogene for sequence similarity is also performed.

Anticipated Problems: One problem relates to the overall accuracy of the sequencing platform. The initial approach is to sequence at ultra-high depth. This approach should allow the determination of non-systematic sequencing errors but inherent errors due to technical constraints of the platform are more difficult to determine. The comparison to the consensus data of the CYP2D6 reference samples allows the estimation of this effect. In addition, it is anticipated that further benchmark studies for the ONT platform and improved sequence analysis methods increase sequence annotation for long-read data.

Future directions: In pharmacogenetics, CYP2D6 stands out as one of the most widely tested genes while being technically challenging to analyze using current testing technologies. The ultimate goal is to develop a unifying clinical testing method that can replace current platforms which are incomplete and error prone. This application serves as proof-of-concept demonstration that CRISPR-based sequence targeting, innovative fragment enrichment and long-read sequencing is a feasible approach.

### Example 5

### Targeting of Specific Genomic Locus for Analysis

This approach uses CRISPR/CAS9 system with locus specific guide RNAs for targeted cutting of region of interest (ROI) only, as compared to traditional methods like PCR or oligonucleotide hybridization. The novel approach of enrichment region selection and sgRNA design allows for the capture of entire gene loci, which include highly similar pseudogenes and repetitive regions, an example of such a region is shown in FIG. 1.

### Current Problem

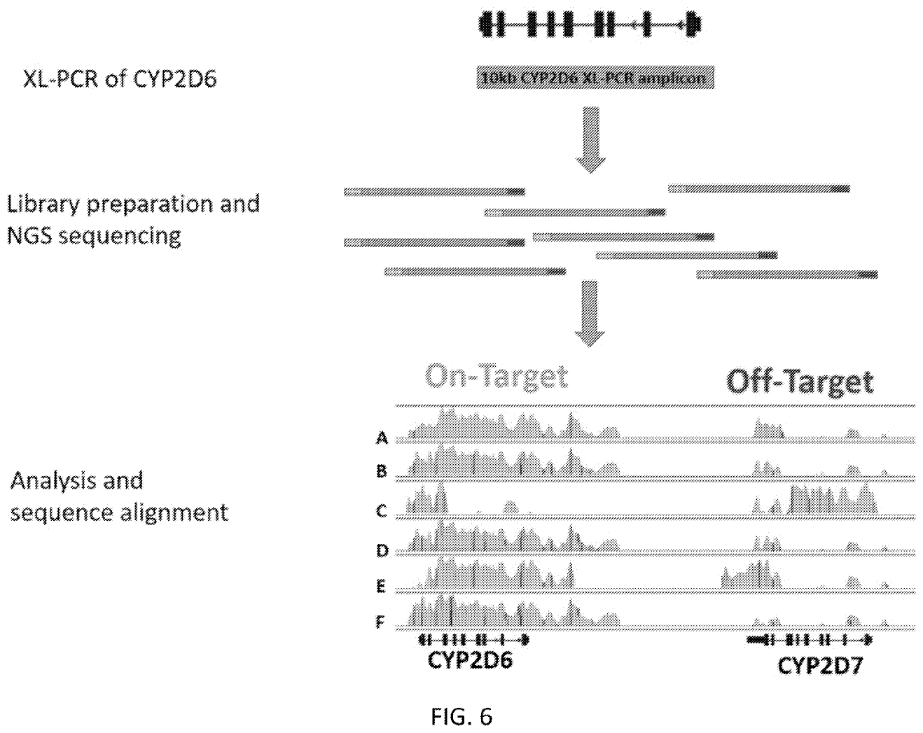

Common DNA extraction methodologies and the sequencing approaches to highly polymorphic genes such as CYP2D6 that include repetitive regions (e.g., REP6, etc.) and share high sequence similarity with neighboring pseudogenes have many weaknesses. These issues include PCR introduced errors, limitations in the size capturable with PCR, off target array hybridization, the need for multiple assays (e.g., ex. sequencing+CNV analysis with qPCR), off target alignment, lack of variant phasing and high monetary and time cost. FIG. 6 highlights IGV alignment of 6 examples of NGS sequenced traditionally prepared libraries. These libraries (A-F) were generated from CYP2D6 long range PCR (XL-PCR) amplicons. The amplicons underwent fragmentation (100-300 bp), adaptor ligation, and PCR amplification prior to NGS analysis. This approach has several limitations. First, as shown for CYP2D6, to amplify the CYP2D6 gene in each sample, the CYP2D6 copy number status and whether a hybrid allele is present or not must be known prior to XL-PCR. Specific primers for normal, duplication, deletion and hybrid alleles must be used for each. This requires an additional copy number assay to be performed prior to NGS. Additionally, XL-PCR amplification time is typically 0.5 to 1 hour per kb length of target amplicon.

The analysis of the short-read sequence data is also hampered by reduced phasing capabilities and is prone to off target alignment to highly similar pseudogene or homologous regions, for example, the CYP2D6 and the 94% similar CYP2D7 pseudogene as shown in FIG. 1. Furthermore, different haplotypes of the same gene can have different levels of similarity with pseudogenes and variants may not be correctly aligned.

The PCR-free libraries have significant benefits over traditional PCR-based approaches. PCR-free libraries remove the potential for the introduction of PCR-derived sequence errors and overcome the current limitations in maximum PCR product size. The XL-PCR reaction time is removed, representing a significant time reduction and the approach allows for heterozygous variant phasing and the detection of copy number variation (CNV).

Design of sgRNAs

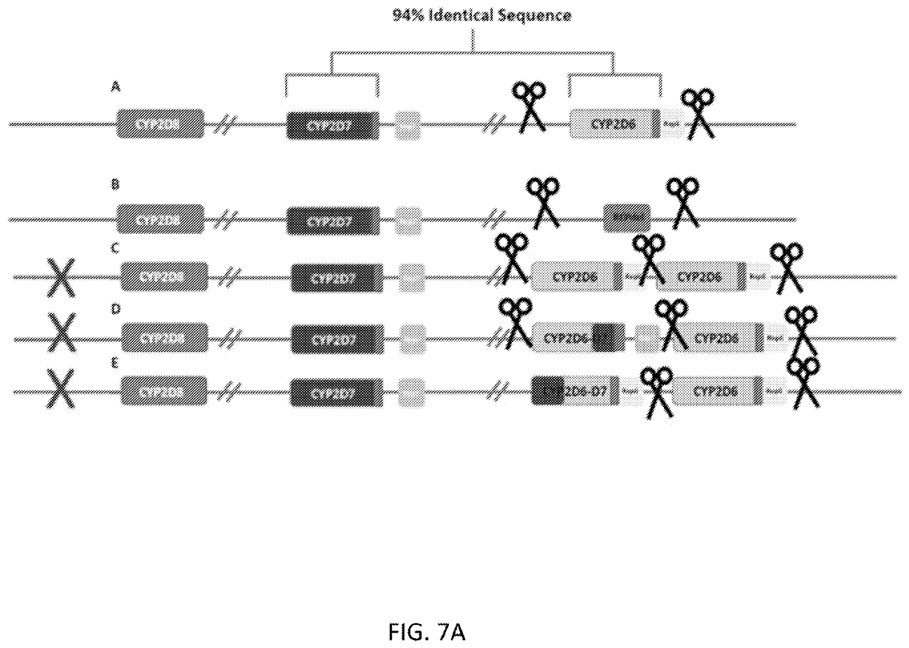

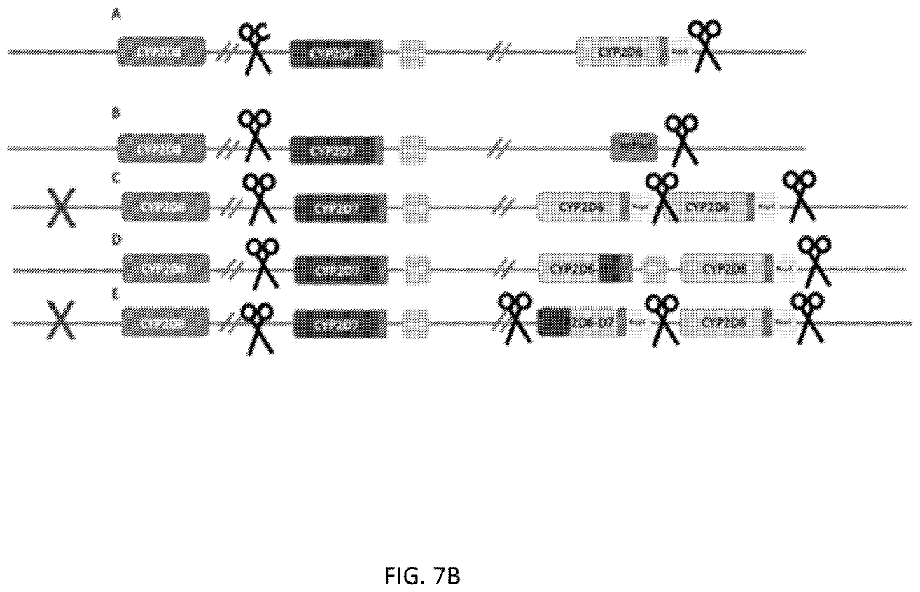

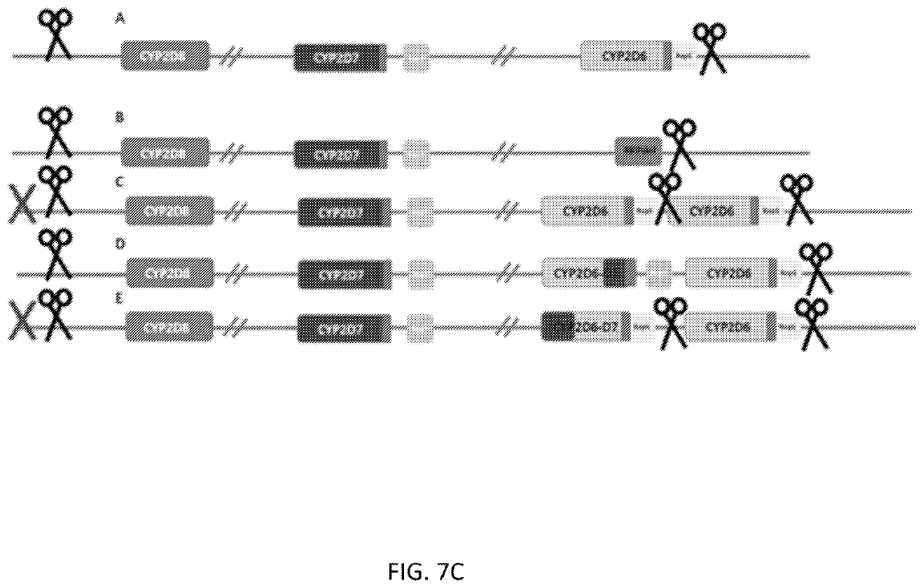

As shown above, due to the complex and highly polymorphic nature of the CYP2D6 loci, traditional PCR and array-based technologies require multiple assays to perform both CNV and SNP analysis. Due to DNA shearing during extraction and sample handling, to maximize the amount of intact target region for enrichment, intuitively the smallest possible CRISPR/Cas9 target region to capture the gene of interested would be selected. However, CRISPR/Cas9 approaches that target only the CYP2D6 gene fail to capture alleles that contain a structural variation, such as a D6/D7 hybrid allele or CYP2D6 duplication events, which make up at least 20% of alleles detected. Examples of the highly complex requirements for appropriate guide RNA design are shown in FIGS. 7A-7C.

The first design limitation is that RNAs to target the Cas9 complex to the ROI cannot be designed near to the CYP2D6 gene itself. This is for two chief regions. The first is that there are limited sites of unique sequence flanking CYP2D6 that are not identical to CYP2D7. Those that are contain repetitive regions that do not work well or are able to capture important promotor region variation. The second reason is that if a CYP2D6 CNV or D6/D7 or D7/D6 hybrid allele is present, there is additional cutting and loss of the ability for accurate CNV analysis and sequence alignment (FIG. 7A). The similar limitations of an approach that cuts close to CYP2D7 and CYP2D8 are shown in FIG. 7B and FIG. 7C, respectively.

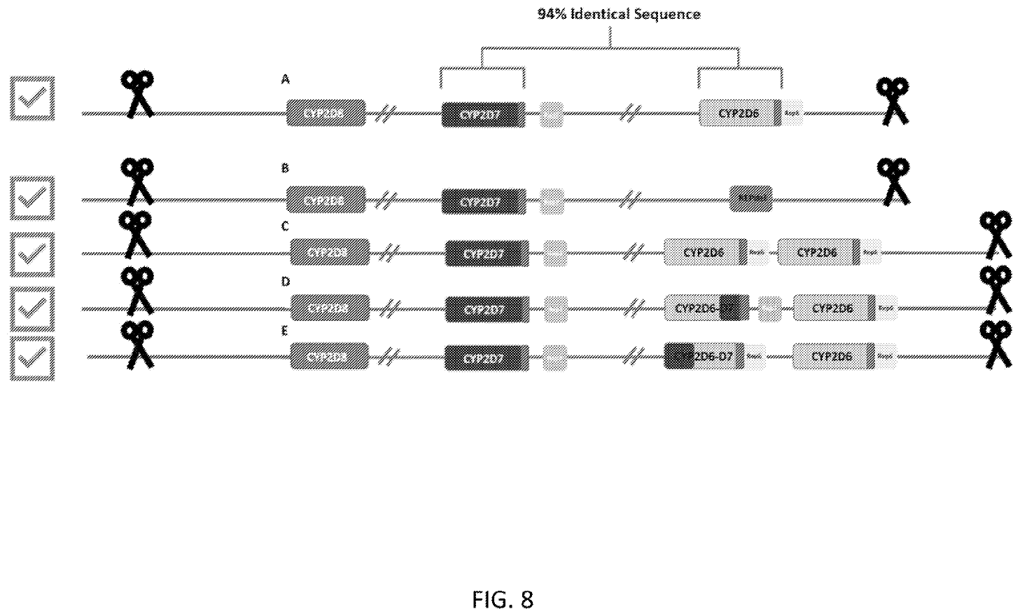

To overcome these limitations, unique sequences that flank the region encompassing both CYP2D6, CYP2D7 and CYP2D8 and still generate a cut fragment of appropriate size for long range sequence analysis have been identified. By designing sgRNAs to target these unique regions, one CRISPR/Cas9 cleavage reaction is performed to isolate the entire CYP2D6/CYP2D7/CYP2D8 region (FIG. 8). Additionally, depending on the downstream application, the design must target the correct strand (+ or −), depending on if the sgRNA targets the 5′ or 3′ end of the ROI. A non-limiting example of sgRNA sequences tested appears in Table 2 below. CYP2D6 is encoded on the − strand, however guide RNA positions (up- or downstream) are referred to relative to the + strand. A sequence with a lower chromosomal position is considered further upstream then a sequence with a higher chromosomal position, which is considered downstream.

TABLE 2
Guide RNA sequences
sgRNA Sequences

| | |
|---|---|
| TCF20_1_1 | AAGGUGGUGGACACUCGUGAGUUUUAGAGCUAGAA |
| (downstream | AUAGCAAGUUAAAAUAAGGCUAGUCCGUUAUCAAC |
| of CYP2D8) | UUGAAAAAGUGGCACCGAGUCGGUGCUUUU |
| | (SEQ ID NO: 1) |
| TCF20_2_1 | CACUAUGGAGAUUGUGUCCAGUUUUAGAGCUAGAA |
| (downstream | AUAGCAAGUUAAAAUAAGGCUAGUCCGUUAUCAAC |
| of CYP2D8) | UUGAAAAAGUGGCACCGAGUCGGUGCUUUU |
| | (SEQ ID NO: 2) |
| NDUFA6_D6_1 | ACGGACACUACCAAGGAGCGGUUUUAGAGCUAGAA |
| (upstream of | AUAGCAAGUUAAAAUAAGGCUAGUCCGUUAUCAAC |
| CYP2D6) | UUGAAAAAGUGGCACCGAGUCGGUGCUUUU |
| | (SEQ ID NO: 3) |
| NDUFA6_D6_2 | CUUGAAGAACCUCCUCGUGGGUUUUAGAGCUAGAA |
| (upstream of | AUAGCAAGUUAAAAUAAGGCUAGUCCGUUAUCAAC |
| CYP2D6) | UUGAAAAAGUGGCACCGAGUCGGUGCUUUU |
| | (SEQ ID NO: 4) |
| N3 | AUGUCUCAAGACUACCCCUCGUUUUAGAGCUAGAA |
| (upstream of | AUAGCAAGUUAAAAUAAGGCUAGUCCGUUAUCAAC |
| CYP2D6) | UUGAAAAAGUGGCACCGAGUCGGUGCUUUU |
| | (SEQ ID NO: 5) |
| AD6_C | CUGUCAUGGGCACGUAGACCGUUUUAGAGCUAGAA |
| (upstream of | AUAGCAAGUUAAAAUAAGGCUAGUCCGUUAUCAAC |
| CYP2D6) | UUGAAAAAGUGGCACCGAGUCGGUGCUUUU |
| | (SEQ ID NO: 6) |
| AD6_D | UCCUCACCGACAUAAUGGGCGUUUUAGAGCUAGAA |
| (upstream of | AUAGCAAGUUAAAAUAAGGCUAGUCCGUUAUCAAC |
| CYP2D6) | UUGAAAAAGUGGCACCGAGUCGGUGCUUUU |
| | (SEQ ID NO: 7) |
| JGYW3632.AA | GGCUUACAAGUUGGUCCUAAGUUUUAGAGCUAGAA |
| (upstream of | AUAGCAAGUUAAAAUAAGGCUAGUCCGUUAUCAAC |
| CYP2D6) | UUGAAAAAGUGGCACCGAGUCGGUGCUUUU |
| | (SEQ ID NO: 8) |
| BJGYW3632.AB | UAUCACCUUUUAGUCAAUUCGUUUUAGAGCUAGAA |
| (upstream of | AUAGCAAGUUAAAAUAAGGCUAGUCCGUUAUCAAC |
| CYP2D6) | UUGAAAAAGUGGCACCGAGUCGGUGCUUUU |
| | (SEQ ID NO: 9) |
| AD6_E | UGUCAAGAAUUAGUGGUGGUGUUUUAGAGCUAGAA |
| (upstream of | AUAGCAAGUUAAAAUAAGGCUAGUCCGUUAUCAAC |
| CYP2D6) | UUGAAAAAGUGGCACCGAGUCGGUGCUUUU |
| | (SEQ ID NO: 10) |
| N4 | CCAUUCACCCUUAUGCUCAGGUUUUAGAGCUAGAA |
| (upstream of | AUAGCAAGUUAAAAUAAGGCUAGUCCGUUAUCAAC |
| CYP2D6) | UUGAAAAAGUGGCACCGAGUCGGUGCUUUU |
| | (SEQ ID NO: 11) |
| N5 | AACCUCCGGUUGCUUCCUGAGUUUUAGAGCUAGAA |
| (upstream of | AUAGCAAGUUAAAAUAAGGCUAGUCCGUUAUCAAC |
| CYP2D6) | UUGAAAAAGUGGCACCGAGUCGGUGCUUUU |
| | (SEQ ID NO: 12) |
| T3 | GGUGGACACUCGUGAUGGAAGUUUUAGAGCUAGAA |
| (downstream of | AUAGCAAGUUAAAAUAAGGCUAGUCCGUUAUCAAC |
| CYP2D8) | UUGAAAAAGUGGCACCGAGUCGGUGCUUUU |
| | (SEQ ID NO: 13) |

crRNA Sequences

| | |
|---|---|
| T3_2 | GGUGGACACUCGUGAUGGAAGUUUUAGAGCUAUGC |
| (downstream | U |
| of CYP2D8) | (SEQ ID NO: 14) |
| TCF20_1_2 | AAGGUGGUGGACACUCGUGAGUUUUAGAGCUAUGC |
| (downstream | U |
| of CYP2D8) | (SEQ ID NO: 15) |
| TCF20_2_2 | CACUAUGGAGAUUGUGUCCAGUUUUAGAGCUAUGC |
| (downstream | U |
| of CYP2D8) | (SEQ ID NO: 16) |
| NDUFA6_D6_1_2 | ACGGACACUACCAAGGAGCGGUUUUAGAGCUAUGC |
| (upstream of | U |
| CYP2D6) | (SEQ ID NO: 17) |
| NDUFA6_D6_2_2 | CUUGAAGAACCUCCUCGUGGGUUUUAGAGCUAUGC |
| (upstream of | U |
| CYP2D6) | (SEQ ID NO: 18) |
| N3_2 | AUGUCUCAAGACUACCCCUCGUUUUAGAGCUAUGC |
| (upstream of | U |
| CYP2D6) | (SEQ ID NO: 19) |
| AD6_C_2 | CUGUCAUGGGCACGUAGACCGUUUUAGAGCUAUGC |
| (upstream of | U |
| CYP2D6) | (SEQ ID NO: 20) |
| AD6_D_2 | UCCUCACCGACAUAAUGGGCGUUUUAGAGCUAUGC |
| (upstream of | U |
| CYP2D6) | (SEQ ID NO: 21) |
| JGYW3632.AA_2 | GGCUUACAAGUUGGUCCUAAGUUUUAGAGCUAUGC |
| (upstream of | U |
| CYP2D6) | (SEQ ID NO: 22) |
| BJGYW3632.AB_2 | UAUCACCUUUUAGUCAAUUCGUUUUAGAGCUAUGC |
| (upstream of | U |
| CYP2D6) | (SEQ ID NO: 23) |
| AD6_E_2 | UGUCAAGAAUUAGUGGUGGUGUUUUAGAGCUAUGC |
| (upstream of | U |
| CYP2D6) | (SEQ ID NO: 24) |
| N4_2 | CCAUUCACCCUUAUGCUCAGGUUUUAGAGCUAUGC |
| (upstream of | U |
| CYP2D6) | (SEQ ID NO: 25) |
| N5_2 | AACCUCCGGUUGCUUCCUGAGUUUUAGAGCUAUGC |
| (upstream of | U |
| CYP2D6) | (SEQ ID NO: 26) |

sgRNA Performance Analysis and Validation

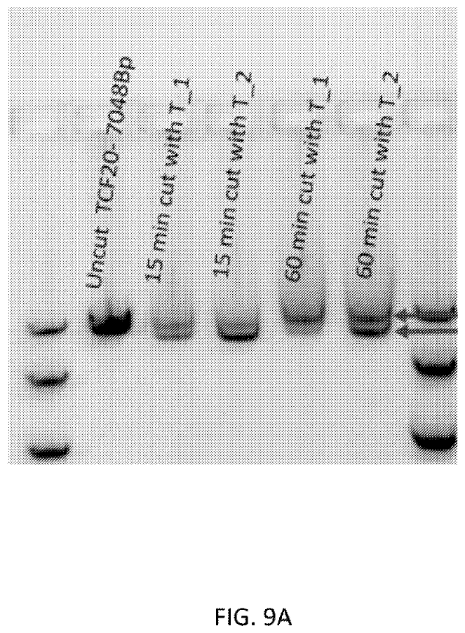

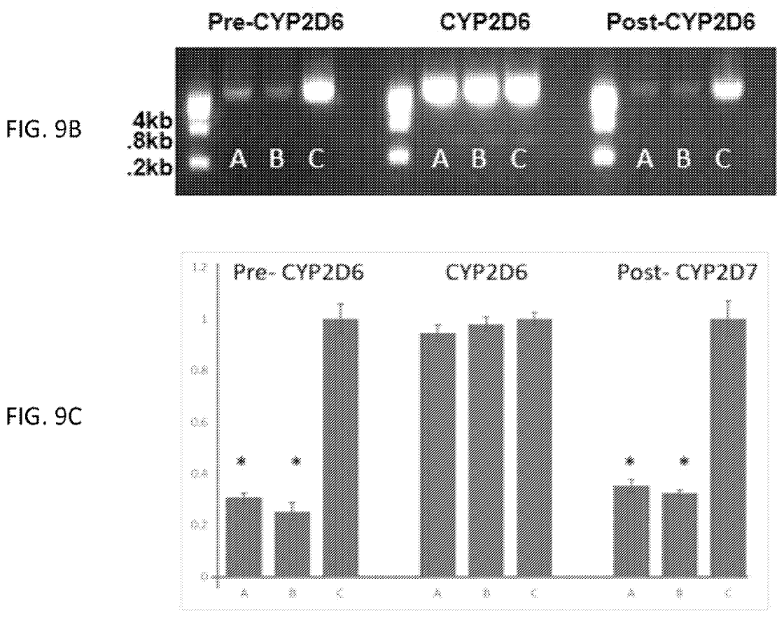

To confirm the specificity and efficacy of the sgRNAs, XL-PCR products that contain the targeted sgRNA binding sites were generated from gDNA. The XL-PCR products were incubated with either Cas9+no sgRNA (or off-target sgRNA) or Cas9+sgRNAs of interest. FIG. 9A shows a representative agarose gel showing the cutting efficiency of two different sgRNAs (T_1 and T_2) at multiple reaction time points. All PCR products incubated with Cas9 and sgRNA were cleaved to produce DNA fragments of the expected size but different sgRNAs showed different degrees of cleavage efficiency.

After the cleavage efficiency of XL-PCR amplicons was determined, the efficiency of cleavage on genomic DNA was analyzed. This was done by performing the Cas-mediated cutting with specific sgRNAs and then performing quantitative PCR reactions on the cut DNA. Primers were designed on either side of the predicted sgRNA target cut sites. PCR reactions were run on 100 ng of total genomic DNA from either the Cas9 reaction or an uncut control. If the DNA was cleaved at the appropriate site, a reduction in PCR product would be observed compared to the amount of PCR product generated in an uncut control sample (e.g., a Cas9 reaction that used sgRNAs for an off target region). Using this approach, it was determined whether the sgRNA was able to target the desired ROI in genomic DNA and the efficiency of that cutting was determined, as shown in FIG. 9B and FIG. 9C. XL-PCR of the entire CYP2D6 gene showed no difference between the cut and uncut control. This indicates that the reduced amount of PCR product observed in the cut site spanning reactions was not due to random cutting of the DNA, but rather targeted Cas9 mediated cutting of those specific regions.

### Isolation of High-Molecular Weight (HMW) DNA

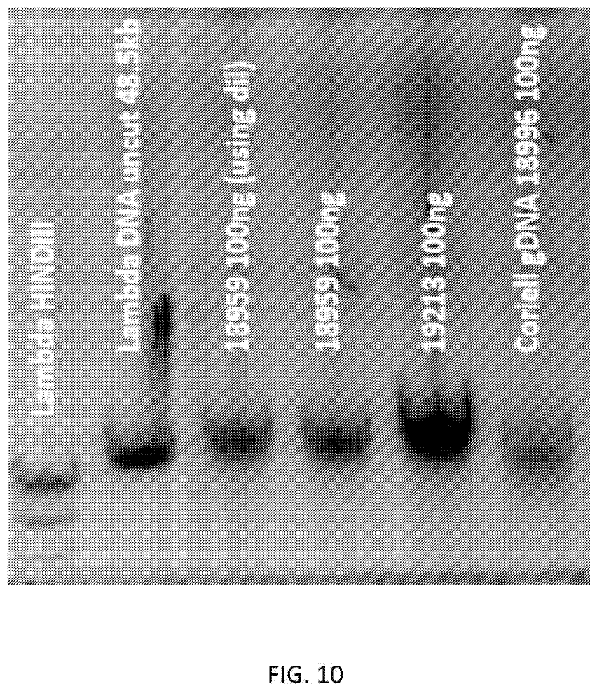

Isolation of high molecular weight genomic (HMW) DNA in long segments (≥50 kb) allows for the generation of sequencing libraries without PCR amplification. As shown in FIG. 10, HMW DNA was extracted in-house from lymphoblast cells (18959 and 19213) using the Nanobind CCB Dig DNA kit (Circulomics, Madison Wi). The extracted DNA was run on a 2% agarose gel and size compared to lambda HINDIII ladder (upper band 23.1 kb), lambda DNA (48.5 kb), and previously extracted genomic DNA acquired from the Corriel Institute (extracted via alternate methodology). The DNA extracted in-house was significantly larger in size than DNA extracted via other methodology (ex. Coriell gDNA 18996), with the majority running above the 48.5 kb lambda DNA. Further enrichment for high molecular weight DNA was done with the Short Read Eliminator Kit (Circulomics, Madison Wi).

### CRISPR/Cas9 Enrichment and Library Preparation

CRISPR/Cas9 enrichment was performed with the above described sgRNAs using a modified version of the Nanopore Cas-mediated protocol (VNR_9084_v109_revK_04Dec2018). Modifications to the volume and concentration of sgRNA used in the process was done to achieve optimal results (specifically, 33.3 μl sgRNA (3 μM) per sgRNA). Adapters were ligated using the Amplicons by Ligation protocol (SQK-LSK109) and the prepared libraries for sequencing were run on the MinION sequencing platform (Oxford Nanopore, UK) and data analysis was performed.

### Proof of Concept

Sequencing utilizing the sgRNAs that enrich for the entire CYP2D6-CYP2D7-CYP2D8 region (chr22: 42, 122, 115-42, 161, 317) confirms 3 key things: (1) The sgRNA designs successfully captures the entire target region, (2) the strategy allows for significant enrichment of the entire ROI over off-target reads and (3) the method results in the ability to successfully long read sequence the entire ROI (˜40 kb).

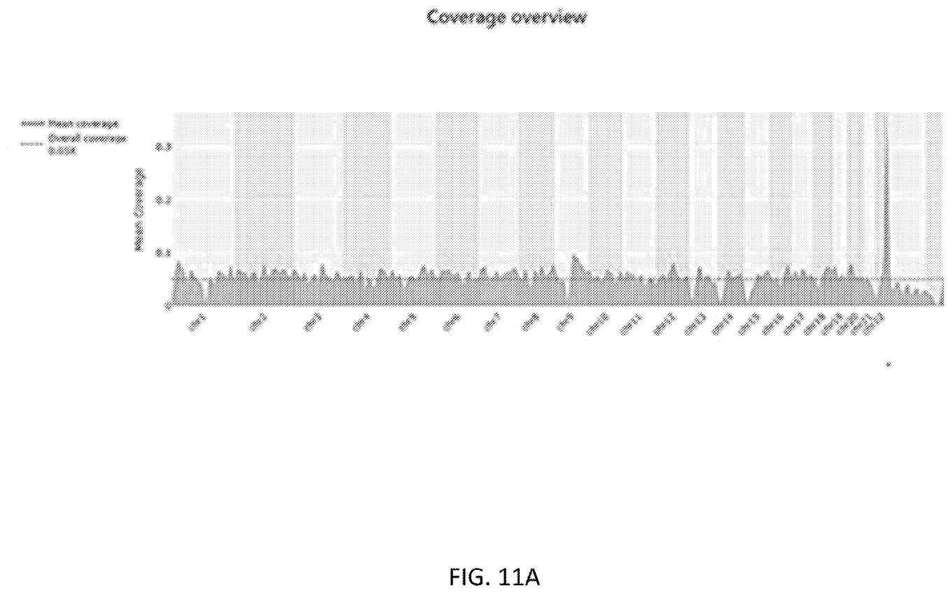

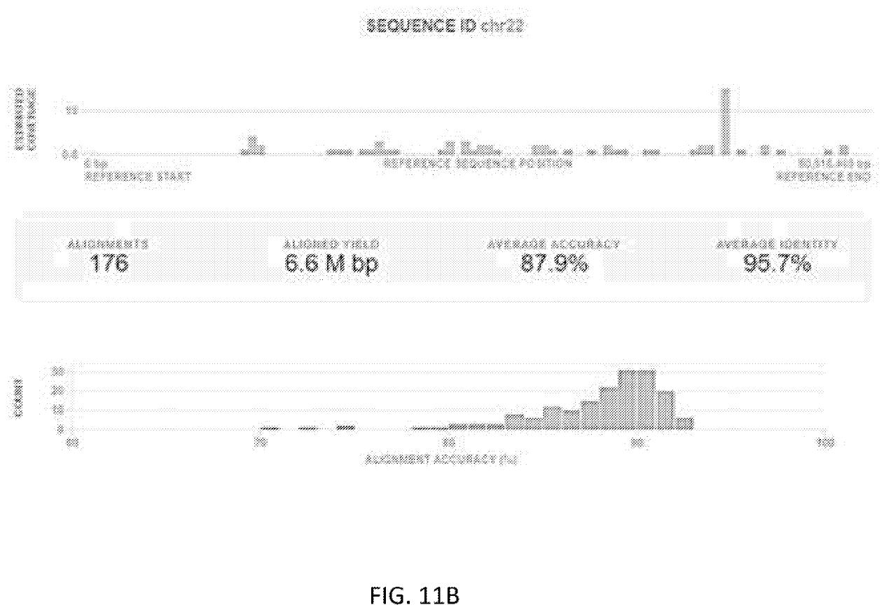

As shown in FIG. 11A, genome wide, significant sequence enrichment was observed for only Chromosome 22 (chr22), which contains the targeted ROI. All other genomic regions showed minimal coverage. Further analysis of chr22 found that only the region containing the ROI was enriched and had >10× coverage (FIG. 11B). In total, 121 of 176 reads mapped to chr22 were full length reads aligning to the ROI (68.75%). The average accuracy and identity per read for all chromosome 22 reads is shown in FIG. 11B.

### Run Alignment and Time

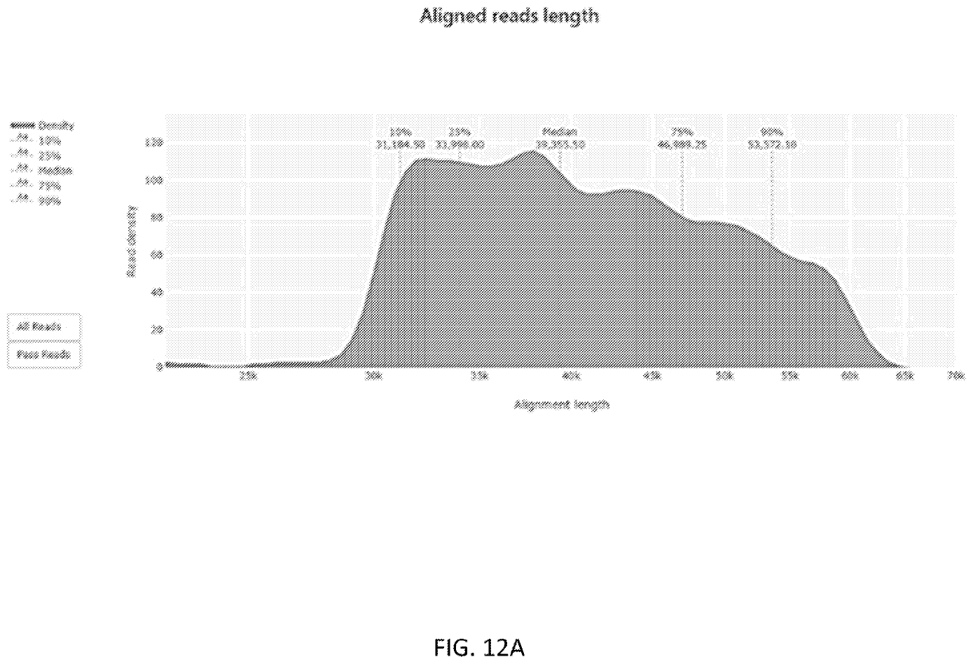

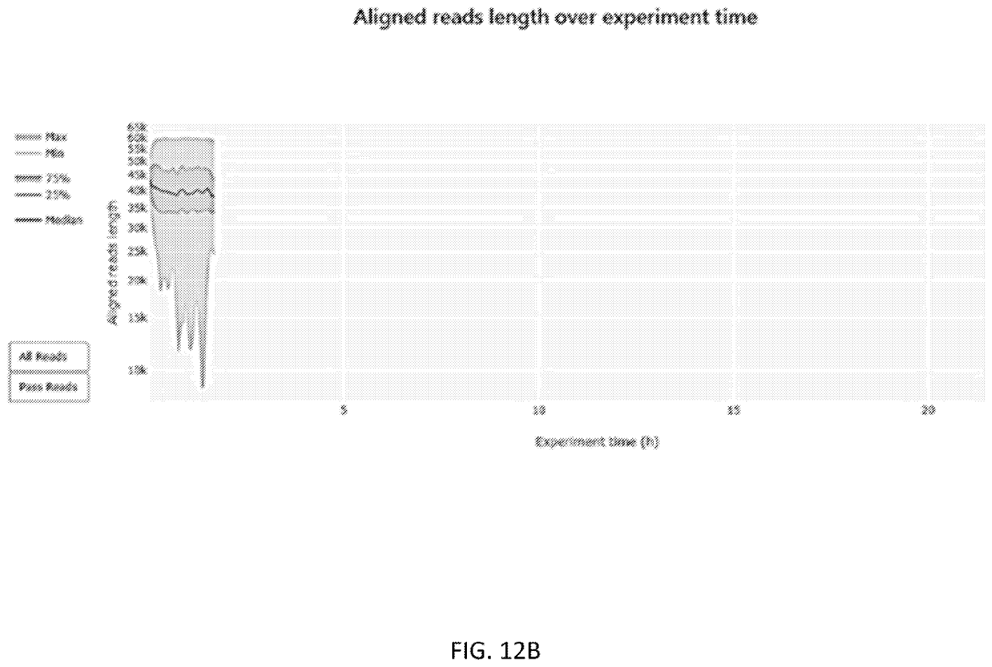

The median aligned read length was ˜39.35 kb (FIG. 12A) indicating successful sequencing and alignment of the target design size. Of note, all reads that aligned were captured in the first 2.5 hours of sequencing on the minION (FIG. 12B). This indicates that sequencing time using the method described herein can be greatly reduced from standard long read sequencing run times. This is of great value, in both results turnaround time and instrument throughput.

### IGV Analysis

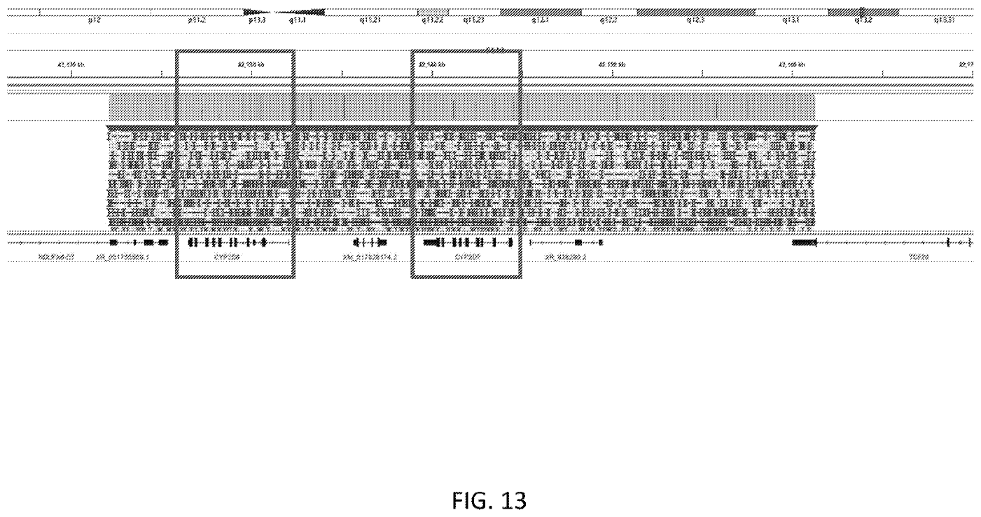

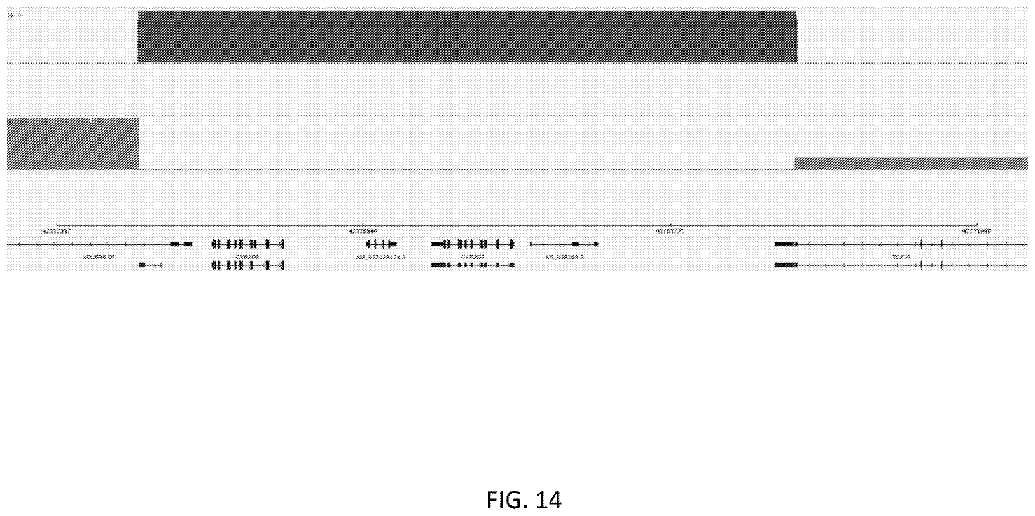

Further IGV analysis of the sequence data alignment showed that the sequence reads aligned to the correct genomic location (chr22: 42, 122, 115-42, 161, 317) and had uniform depth and coverage across the entire ROI. FIG. 13 shows IGV alignment of 121 38.5 kb reads aligning to the target CYP2D6 region. To further review the specificity of the approach, sgRNA enrichment in the target region, but of the opposite DNA strands (+ or −) was performed and sequence data alignment was compared to the sgRNA enrichment on the original strand design. As shown in FIG. 14, 100% sequence enrichment was generated in the ROIs, either CYP2D6-CYP2D7-CYP2D8 region (chr22: 42, 122, 115-42, 161, 317—shown in the upper alignment in the figure) or the flanking regions (shown in the lower alignment in the figure), depending on the sgRNA strand target. No overlap with flanking off target regions was observed, depending on the design. This demonstrates two critical aspects of the approach: (1) significant off target cutting within our design ROI is not generated, and (2) the enrichment approach does not lead to significant shearing of the ROI.

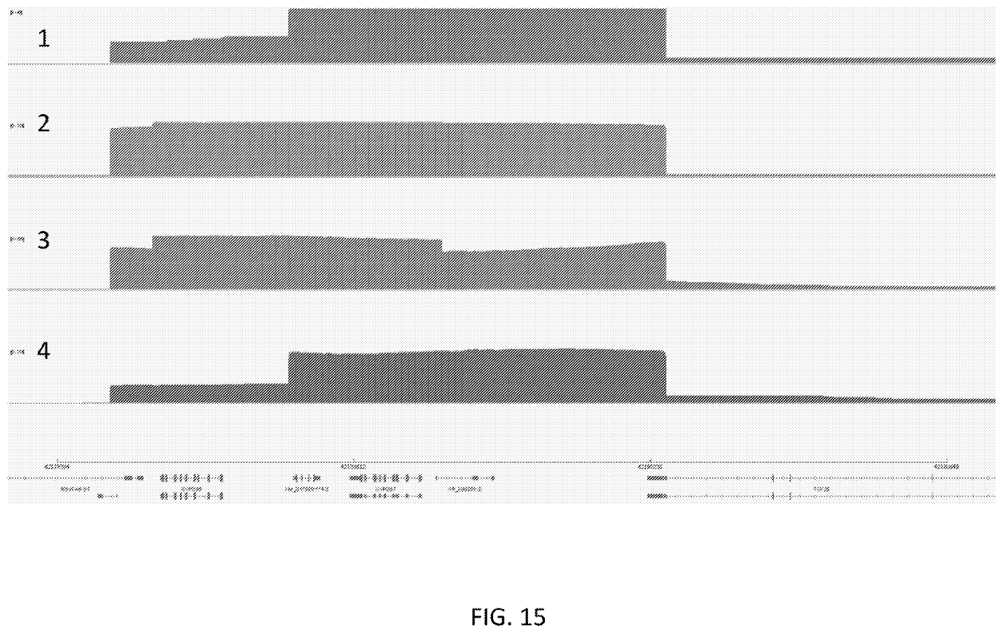

FIG. 15 depicts a Sashimi plot showing sgRNA specificity for multiple complex structural arrangements. This plot shows the aligned region for four sequencing runs. The sequence data from the runs uses the sgRNAs designed to capture the region-of-interest (ROI) (chr22:42, 122, 115-41, 161, 320) and includes four different structural events: (1) Deletion of CYP2D6 on one allele; (2) Hybrid allele in tandem with CYP2D6 on one allele; (3) Duplication event on one allele; and (4) Deletion of CYP2D6 on one allele and duplication of CYP2D6 on the second allele. This data represents successful enrichment of structural variations for the ROI for all orientations of recombination, including a CYP2D6 CNV or D6/D7 or D7/D6 hybrid allele, including those with upstream CYP2D6-like or CYP2D7-like regions and those with CYP2D6-like or CYP2D7-like downstream regions. No off-target cutting between the regions upstream of CYP2D6 and downstream of CYP2D8 occurred regardless of the structural variation present, overcoming the limitations in design described in FIG. 7 and confirming the approach described in FIG. 8.

### Example 6. Nested CRISPR-Cas9 Method for Enriching Genomic Region of Interest

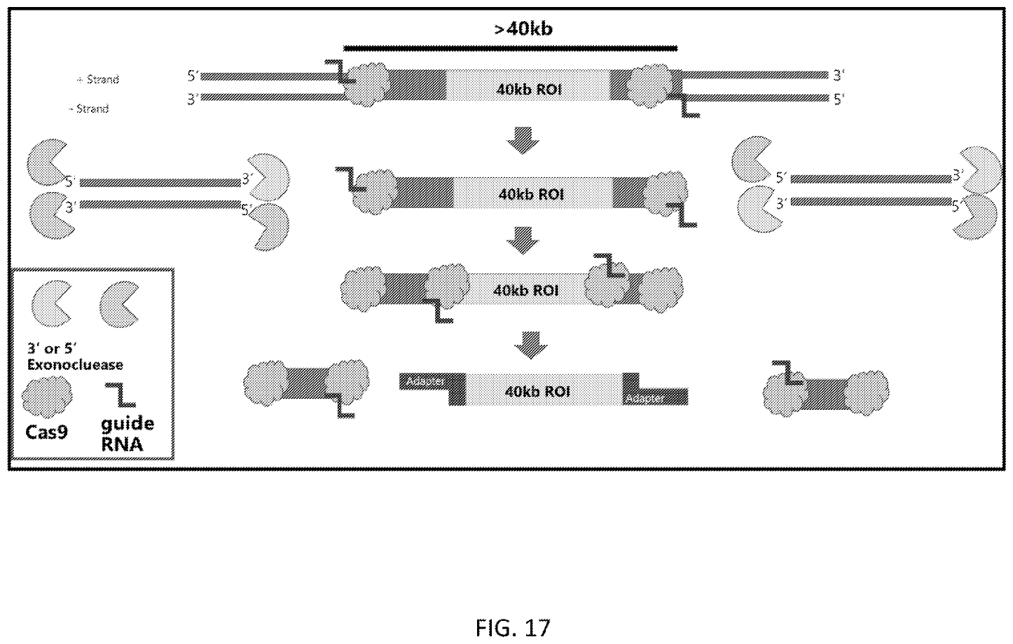

In this example, a nested CRISPR-Cas9 approach is used to enrich for (e.g., complex) genomic regions of interest. This approach has numerous benefits over current approaches including: (1) increased specificity of enrichment for the region of interest; and (2) increased capacity of input DNA material to increase the overall enrichment of the ROI. FIG. 17 provides an example schematic for performing a nested enrichment as described herein.

In this example, a CRISPR-Cas9 reaction is performed using as much genomic DNA as is desired for downstream use. An outer set of guide RNAs is designed that are up to 30 kb downstream and upstream of the targeted region of interest (e.g., CYP2D6 locus). The Cas9-guide RNA complex cuts the genomic region of interest from the genomic DNA and blocks the ends of the excised DNA fragment containing the region of interest. An exonuclease digest is then performed, digesting the unprotected DNA (e.g., the DNA that does not contain the region of interest). Because the ends of the DNA fragments containing the genomic region of interest are protected from exonuclease digestion (e.g., by steric hindrance due to the bound Cas9-guide RNA complexes), the excised DNA fragments containing the region of interest are left intact. This step allows for both an additional enrichment for the region of interest that increases specificity and the ability to use larger amount of genomic DNA (e.g., >10 μg) than typically used during Cas-based enrichment protocols.

After the exonuclease digestion is performed, the enriched large undigested fragments are used in a CRISPR-Cas9 reaction using an inner set of guide RNAs that targets the desired region of interest of the appropriate size for long-read sequencing. This step adds further specificity to the first enrichment protocol and fees up the ends of the region of interest for downstream library generation.

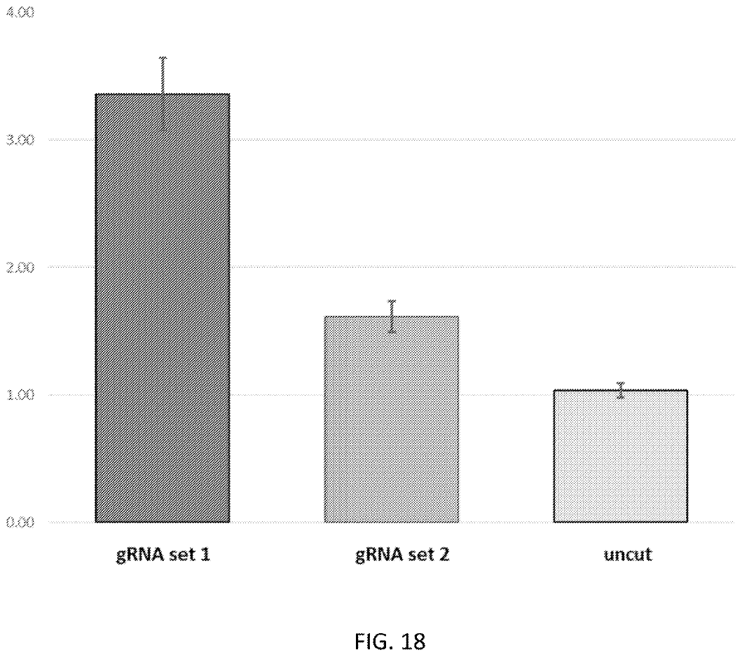

The efficiency of the nested CRISPR-Cas9 approach is shown in FIG. 18 for two representative sets of sgRNAs. As shown in FIG. 18, two representative sets of outer gRNAs located either 10 kb (set 1) or 20 kb (set 2) upstream of the inner gRNA cut sites were used to perform initial enrichment. The uncut sample received no outer gRNA enrichment. The same set of inner gRNAs were then used on set 1, set 2, and uncut samples and libraries were prepared as described above. As shown in FIG. 18, the fold enrichment observed over uncut was approximately 1.7 fold for set 2, and approximately 3.4 fold for set 1.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the embodiments of the disclosure. It is intended that the following claims define the scope of the disclosure and that methods and structures within the scope of these claims and their equivalents be covered thereby.

What is claimed is:

1. A method of analyzing (e.g., sequencing, genotyping, structural analysis) a genomic region of interest, said method comprising:
a) contacting genomic DNA comprising said genomic region of interest with a Clustered Regularly Interspaced Short Palindromic Repeat (CRISPR)-associated endonuclease and an outer pair of guide RNAs (gRNAs), thereby generating a first excised fragment comprising said genomic region of interest;
b) contacting said first excised fragment with a CRISPR-associated endonuclease and an inner pair of gRNAs, thereby generating a second excised fragment comprising said genomic region of interest; and
c) analyzing said genomic region of interest contained within said second excised fragment.

2. The method of claim 1, wherein said CRISPR-associated endonuclease and said outer pair of gRNAs of a) associate with and block the 5′ and 3′ ends of said first excised fragment.

3. The method of claim 2, further comprising, prior to b), contacting the product of a) with one or more exonucleases, such that background genomic DNA is digested and said first excised fragment is not digested.

4. The method of any one of the preceding claims, wherein said one or more exonucleases are selected from the group consisting of: exonuclease I, exonuclease II, exonuclease III, exonuclease IV, exonuclease V, exonuclease VI, exonuclease VII, exonuclease VIII, and any combination thereof.

5. The method of any one of the preceding claims, wherein said outer pair of gRNAs comprises a first outer gRNA and a second outer gRNA.

6. The method of claim 5, wherein said first outer gRNA comprises a nucleotide sequence that is substantially complementary to a first nucleotide sequence present in said genomic DNA, and said second outer gRNA comprises a nucleotide sequence that is substantially complementary to a second nucleotide sequence present in said genomic DNA.

7. The method of claim 6, wherein said first nucleotide sequence and said second nucleotide sequence are different.

8. The method of claim 7, wherein said first nucleotide sequence and said second nucleotide sequence flank said genomic region of interest.

9. The method of claim 8, wherein said first nucleotide sequence, said second nucleotide sequence, or both, are present in said genomic DNA up to about 100 kilobases in length from said genomic region of interest.

10. The method of any one of the preceding claims, wherein said inner pair of gRNAs comprises a first inner gRNA and a second inner gRNA.

11. The method of claim 10, wherein said first inner gRNA comprises a nucleotide sequence that is substantially complementary to a third nucleotide sequence present in said genomic DNA, and said second inner gRNA comprises a nucleotide sequence that is substantially complementary to a fourth nucleotide sequence present in said genomic DNA.

12. The method of claim 11, wherein said third nucleotide sequence and said fourth nucleotide sequence are different.

13. The method of claim 12, wherein said third nucleotide sequence and said fourth nucleotide sequence flank said genomic region of interest.

14. The method of any one of claim 6-9 or 11-13, wherein said third nucleotide sequence and said fourth nucleotide sequence are present on said genomic DNA at a base length closer to said genomic region of interest than said first nucleotide sequence and said second nucleotide sequence.

15. The method of any one of the preceding claims, wherein said second excised fragment is smaller in base length than said first excised fragment.

16. The method of claim 1, wherein said analyzing comprises sequencing said genomic region of interest contained within said second excised fragment.

17. The method of any one of the preceding claims, wherein said genomic DNA is provided at an amount of about 10 μg or greater.

18. The method of any one of the preceding claims, wherein said analyzing comprises genotyping said genomic region of interest contained within said second excised fragment.

19. The method of any one of the preceding claims, wherein said analyzing comprises performing structural analysis on said genomic region of interest contained within said second excised fragment.

20. The method of any one of the preceding claims, further comprising, prior to b), isolating said first excised fragment.

21. The method of any one of the preceding claims, further comprising, prior to c), isolating said second excised fragment.

22. The method of any one of the preceding claims, wherein said method does not involve DNA amplification.

23. The method of any one of the preceding claims, further comprising, prior to c), attaching one or more adapters to the 5′ end, the 3′ end, or both, of said second excised fragment.

24. The method of any one of the preceding claims, wherein said CRISPR-associated endonuclease is a Class 1 CRISPR-associated endonuclease or a Class 2 CRISPR-associated endonuclease.

25. The method of claim 24, wherein said Class 1 CRISPR-associated endonuclease is selected from the group consisting of: Cas3, Cas5, Cas8a, Cas8b, Cas8c, Cas10d, Cse1, Cse2, Csy1, Csy2, Csy3, GSU0054, Cas10, Csm2, Cmr5, Csx11, Csx10, and Csf1.

26. The method of claim 24, wherein said Class 2 CRISPR-associated endonuclease is selected from the group consisting of: Cas9, Cas12a, Csn2, Cas4, Cas12b, Cas12c, Cas13a, Cas13b, Cas13c, and Cas13d.

27. The method of any one of the preceding claims, wherein said CRISPR-associated endonuclease comprises an amino acid sequence having at least 80% sequence identity to a wild-type CRISPR-associated endonuclease.

28. The method of any one of the preceding claims, wherein said CRISPR-associated endonuclease is Cas9 or a variant thereof.

29. The method of claim 28, wherein said Cas9 is a Streptococcus pyogenes Cas9 (spCas9).

30. The method of claim 28 or 29, wherein said Cas9 variant comprises one or more point mutations, relative to a wild-type Streptococcus pyogenes Cas9 (spCas9), selected from the group consisting of: R780A, K810A, K848A, K855A, H982A, K1003A, R1060A, D1135E, N497A, R661A, Q695A, Q926A, L169A, Y450A, M495A, M694A, and M698A.

31. The method of any one of the preceding claims, wherein said genomic DNA is not fragmented, digested, or sheared prior to a).

32. The method of any one of the preceding claims, wherein said genomic DNA is not subjected to restriction enzyme digestion prior to a).

33. The method of any one of the preceding claims, wherein said genomic region of interest is a complex genomic region.

34. The method of claim 33, wherein said complex genomic region comprises a gene of interest and one or more pseudogenes thereof.

35. The method of claim 34, wherein said one or more pseudogenes comprise a nucleotide sequence having at least 75% sequence identity to said gene of interest.

36. The method of any one of claim 33, wherein said complex genomic region comprises one or more repetitive regions, one or more duplications, one or more insertions, one or more inversions, one or more tandem repeats, one or more retrotransposons, or any combination thereof.

37. The method of any one of the preceding claims, wherein said genomic region of interest is a highly polymorphic gene locus.

38. The method of any one of the preceding claims, wherein said first excised fragment is at least about 0.06 kilobases in length.

39. The method of any one of the preceding claims, wherein said first excised fragment is up to about 200 kilobases in length.

40. The method of any one of the preceding claims, wherein said second excised fragment is at least about 0.02 kilobases in length.

41. The method of any one of the preceding claims, wherein said second excised fragment is up to about 199.98 kilobases in length.

42. The method of any one of the preceding claims, wherein said sequencing comprises long-read sequencing.

43. The method of claim 42, wherein said long-read sequencing comprises single-molecule real-time sequencing or nanopore sequencing.

44. The method of any one of the preceding claims, wherein said method does not involve any one of polymerase chain reaction (PCR) or isothermal amplification.

45. The method of claim 44, wherein said method does not involve any one of multiple displacement amplification (MDA), strand displacement amplification (SDA), nucleic acid sequence based amplification (NASBA), loop-mediated isothermal amplification, rolling circle amplification (RCA), ligase chain reaction (LCR), helicase dependent amplification, or ramification amplification method.

46. The method of any one of the preceding claims, wherein said genomic DNA is provided or obtained in a biological sample.

47. The method of claim 46, wherein said biological sample comprises a body fluid (e.g., blood (e.g., whole blood, plasma, serum), urine, saliva, bone marrow, spinal fluid, sputum, ascites, lymphatic fluid, pleural fluid, amniotic fluid, semen, vaginal fluid, sweat, stool, glandular secretions, ocular fluids, breast milk) or a solid tissue sample.

48. The method of claim 47, wherein said biological sample is a diagnostic sample.

49. The method of any one of the preceding claims, wherein said genomic region of interest is a genetic locus comprising CYP2D6, CYP2D7, and CYP2D8.

50. The method of claim 49, wherein said analyzing comprises identifying one or more genetic variations in CYP2D6.

51. The method of claim 50, further comprising, identifying a subject as having a reduction, a loss of, or an increase in CYP2D6 function based on said genetic variation.

52. The method of claim 51, further comprising, recommending a treatment or an alternative treatment to said subject based on said identifying.

53. The method of claim 51, wherein, when said subject is identified as having a reduction in, a loss of, or an increase in CYP2D6 function, recommending an alternative treatment to said subject.

54. The method of claim 51, further comprising, recommending a dosage of a therapeutic to said subject based on said identifying.

55. The method of claim 51, wherein, when said subject is identified as having a reduction in, a loss of, or an increase in CYP2D6 function, altering a dosage of a therapeutic.

56. The method of any one of the preceding claims, wherein said outer pair of gRNAs, said inner pair of gRNAs, or both, comprise gRNAs selected from any one of SEQ ID NOS: 1-418.

57. A kit for analyzing a genomic region of interest, said kit comprising:
a) a Clustered Regularly Interspaced Short Palindromic Repeat (CRISPR)-associated endonuclease;
b) an outer pair of gRNAs comprising:
i) a first outer gRNA comprising a nucleotide sequence that is substantially complementary to a first nucleotide sequence present in genomic DNA that is upstream of said genomic region of interest; and
ii) a second outer gRNA comprising a nucleotide sequence that is substantially complementary to a second nucleotide sequence present in genomic DNA that is downstream of said genomic region of interest;
c) an inner pair of gRNAs comprising:
iii) a first inner gRNA comprising a nucleotide sequence that is substantially complementary to a third nucleotide sequence present in genomic DNA that is upstream of said genomic region of interest; and
iv) a second inner gRNA comprising a nucleotide sequence that is substantially complementary to a fourth nucleotide sequence present in genomic DNA that is downstream of said genomic region of interest,
wherein said third nucleotide sequence and said fourth nucleotide sequence are present on said genomic DNA at a base length closer to said genomic region of interest than said first nucleotide sequence and said second nucleotide sequence.

58. The kit of claim 57, further comprising, one or more exonucleases.

59. The kit of claim 58, wherein said one or more exonucleases are selected from the group consisting of: exonuclease I, exonuclease II, exonuclease III, exonuclease IV, exonuclease V, exonuclease VI, exonuclease VII, exonuclease VIII, and any combination thereof.

60. The kit of any one of claims 57-59, wherein said CRISPR-associated endonuclease is a Class 1 or a Class 2 CRISPR-associated endonuclease.

61. The kit of claim 60, wherein said Class 1 CRISPR-associated endonuclease is selected from the group consisting of: Cas3, Cas5, Cas8a, Cas8b, Cas8c, Cas10d, Cse1, Cse2, Csy1, Csy2, Csy3, GSU0054, Cas10, Csm2, Cmr5, Csx11, Csx10, and Csf1.

62. The kit of claim 60, wherein said Class 2 CRISPR-associated endonuclease is selected from the group consisting of: Cas9, Cas12a, Csn2, Cas4, Cas12b, Cas12c, Cas13a, Cas13b, Cas13c, and Cas13d.

63. The kit of any one of claims 57-62, wherein said CRISPR-associated endonuclease comprises an amino acid sequence having at least 80% sequence identity to a wild-type CRISPR-associated endonuclease.

64. The kit of any one of claims 57-63, wherein said CRISPR-associated endonuclease is Cas9 or a variant thereof.

65. The kit of claim 64, wherein said Cas9 is a Streptococcus pyogenes Cas9 (spCas9).

66. The kit of claim 64 or 65, wherein said Cas9 variant comprises one or more point mutations, relative to a wild-type Streptococcus pyogenes Cas9 (spCas9), selected from the group consisting of: R780A, K810A, K848A, K855A, H982A, K1003A, R1060A, D1135E, N497A, R661A, Q695A, Q926A, L169A, Y450A, M495A, M694A, and M698A.

67. The kit of any one of claims 57-66, wherein said genomic region of interest is a genomic locus comprising CYP2D6, CYP2D7, and CYP2D8.

68. The kit of claim 67, wherein said first outer guide RNA, said first inner guide RNA, or both, comprise the nucleotide sequence of any one of SEQ ID NOS: 3-12, 17-26, 68-77, 82-214, and 344-418.

69. The kit of claim 67 or 68, wherein said second outer guide RNA, said second inner guide RNA, or both, comprise the nucleotide sequence of any one of SEQ ID NOS: 1, 2, 13-16, 27-67, 78-81, and 215-343.

70. The kit of any one of claims 57-69, further comprising, instructions for using said kit in a nested CRISPR reaction.

71. The kit of any one of claims 57-70, further comprising, instructions for using said kit to excise said genomic region of interest from genomic DNA.

72. A system for analyzing a genomic region of interest, said system comprising:
(a) at least one memory location configured to receive a data input comprising data generated from a method comprising:
(i) contacting genomic DNA comprising said genomic region of interest with a Clustered Regularly Interspaced Short Palindromic Repeat (CRISPR)-associated endonuclease and an outer pair of guide RNAs (gRNAs), thereby generating a first excised fragment comprising said genomic region of interest;
(ii) contacting said first excised fragment with a CRISPR-associated endonuclease and an inner pair of gRNAs, thereby generating a second excised fragment comprising said genomic region of interest; and
(iii) analyzing said genomic region of interest contained within said second excised fragment; and
(b) a computer processor operably coupled to said at least one memory location, wherein said computer processor is programmed to generate an output based on said data.

73. The system of claim 72, wherein said output is a report.

74. The system of claim 72 or 73, wherein said output is a genotype of said genomic region of interest.

75. The system of claim 72 or 73, wherein said output is a genetic sequence of said genomic region of interest.

76. The system of claim 72 or 73, wherein said output is a structural analysis of said genomic region of interest.

77. The system of any one of claims 72-76, wherein said analyzing comprises genotyping said genomic region of interest.

78. The system of any one of claims 72-77, wherein said analyzing comprises performing structural analysis of said genomic region of interest.

79. The system of any one of claims 72-78, wherein said analyzing comprises sequencing said genomic region of interest.

80. The system of claim 79, wherein said sequencing comprises long-read sequencing.

81. The system of claim 80, wherein said long-read sequencing comprises single-molecule real-time sequencing or nanopore sequencing.

82. The system of any one of claims 72-81, wherein said CRISPR-associated endonuclease and said outer pair of gRNAs of (i) associate with and block the 5′ and 3′ ends of said first excised fragment.

83. The system of claim 82, further comprising, prior to (ii), contacting the product of (i) with one or more exonucleases, such that background genomic DNA is digested and said first excised fragment is not digested.

84. The system of any one of claims 72-83, wherein said one or more exonucleases are selected from the group consisting of: exonuclease I, exonuclease II, exonuclease III, exonuclease IV, exonuclease V, exonuclease VI, exonuclease VII, exonuclease VIII, and any combination thereof.

85. The system of any one of claims 72-84, wherein said outer pair of gRNAs comprises a first outer gRNA and a second outer gRNA.

86. The system of claim 85, wherein said first outer gRNA comprises a nucleotide sequence that is substantially complementary to a first nucleotide sequence present in said genomic DNA, and said second outer gRNA comprises a nucleotide sequence that is substantially complementary to a second nucleotide sequence present in said genomic DNA.

87. The system of claim 86, wherein said first nucleotide sequence and said second nucleotide sequence are different.

88. The system of claim 87, wherein said first nucleotide sequence and said second nucleotide sequence flank said genomic region of interest.

89. The system of claim 88, wherein said first nucleotide sequence, said second nucleotide sequence, or both, are present in said genomic DNA up to about 100 kilobases in length from said genomic region of interest.

90. The system of any one of claims 72-89, wherein said inner pair of gRNAs comprises a first inner gRNA and a second inner gRNA.

91. The system of claim 90, wherein said first inner gRNA comprises a nucleotide sequence that is substantially complementary to a third nucleotide sequence present in said genomic DNA, and said second inner gRNA comprises a nucleotide sequence that is substantially complementary to a fourth nucleotide sequence present in said genomic DNA.

92. The system of claim 91, wherein said third nucleotide sequence and said fourth nucleotide sequence are different.

93. The system of claim 92, wherein said third nucleotide sequence and said fourth nucleotide sequence flank said genomic region of interest.

94. The system of any one of claims 91-93, wherein said third nucleotide sequence and said fourth nucleotide sequence are present on said genomic DNA at a base length closer to said genomic region of interest than said first nucleotide sequence and said second nucleotide sequence.

95. The system of any one of claims 72-94, wherein said second excised fragment is smaller in base length than said first excised fragment.

96. The system of any one of claims 72-95, wherein said analyzing comprises sequencing said genomic region of interest contained within said second excised fragment.

97. The system of any one of claims 72-96, wherein said genomic DNA is provided at an amount of about 10 μg or greater.

98. The system of any one of claims 72-97, wherein said analyzing comprises genotyping said genomic region of interest contained within said second excised fragment.

99. The system of any one of claims 72-98, wherein said analyzing comprises performing structural analysis on said genomic region of interest contained within said second excised fragment.

100. The system of any one of claims 72-99, further comprising, prior to (ii), isolating said first excised fragment.

101. The system of any one of claims 72-100, further comprising, prior to (iii), isolating said second excised fragment.

102. The system of any one of claims 72-101, wherein said method does not involve DNA amplification.

103. The system of any one of claims 72-102, further comprising, prior to (iii), attaching one or more adapters to the 5′ end, the 3′ end, or both, of said second excised fragment.

104. The system of any one of claims 72-103, wherein said CRISPR-associated endonuclease is a Class 1 CRISPR-associated endonuclease or a Class 2 CRISPR-associated endonuclease.

105. The system of claim 104, wherein said Class 1 CRISPR-associated endonuclease is selected from the group consisting of: Cas3, Cas5, Cas8a, Cas8b, Cas8c, Cas10d, Cse1, Cse2, Csy1, Csy2, Csy3, GSU0054, Cas10, Csm2, Cmr5, Csx11, Csx10, and Csf1.

106. The system of claim 104, wherein said Class 2 CRISPR-associated endonuclease is selected from the group consisting of: Cas9, Cas12a, Csn2, Cas4, Cas12b, Cas12c, Cas13a, Cas13b, Cas13c, and Cas13d.

107. The system of any one of claims 72-106, wherein said CRISPR-associated endonuclease comprises an amino acid sequence having at least 80% sequence identity to a wild-type CRISPR-associated endonuclease.

108. The system of any one of claims 72-107, wherein said CRISPR-associated endonuclease is Cas9 or a variant thereof.

109. The system of claim 108, wherein said Cas9 is a Streptococcus pyogenes Cas9 (spCas9).

110. The system of claim 108 or 109, wherein said Cas9 variant comprises one or more point mutations, relative to a wild-type Streptococcus pyogenes Cas9 (spCas9), selected from the group consisting of: R780A, K810A, K848A, K855A, H982A, K1003A, R1060A, D1135E, N497A, R661A, Q695A, Q926A, L169A, Y450A, M495A, M694A, and M698A.

111. The system of any one of claims 72-110, wherein said genomic DNA is not fragmented, digested, or sheared prior to (i).

112. The system of any one of claims 72-111, wherein said genomic DNA is not subjected to restriction enzyme digestion prior to (i).

113. The system of any one of claims 72-112, wherein said genomic region of interest is a complex genomic region.

114. The system of claim 113, wherein said complex genomic region comprises a gene of interest and one or more pseudogenes thereof.

115. The system of claim 114, wherein said one or more pseudogenes comprise a nucleotide sequence having at least 75% sequence identity to said gene of interest.

116. The system of claim 113, wherein said complex genomic region comprises one or more repetitive regions, one or more duplications, one or more insertions, one or more inversions, one or more tandem repeats, one or more retrotransposons, or any combination thereof.

117. The system of any one of claims 72-116, wherein said genomic region of interest is a highly polymorphic gene locus.

118. The system of any one of claims 72-117, wherein said first excised fragment is at least about 0.06 kilobases in length.

119. The system of any one of claims 72-118, wherein said first excised fragment is up to about 200 kilobases in length.

120. The system of any one of claims 72-119, wherein said second excised fragment is at least about 0.02 kilobases in length.

121. The system of any one of claims 72-120, wherein said second excised fragment is up to about 199.98 kilobases in length.

122. The system of any one of claims 72-121, wherein said method does not involve any one of polymerase chain reaction (PCR) or isothermal amplification.

123. The system of claim 122, wherein said method does not involve any one of multiple displacement amplification (MDA), strand displacement amplification (SDA), nucleic acid sequence based amplification (NASBA), loop-mediated isothermal amplification, rolling circle amplification (RCA), ligase chain reaction (LCR), helicase dependent amplification, or ramification amplification method.

124. The system of any one of the claims 72-123, wherein said genomic DNA is provided or obtained in a biological sample.

125. The system of claim 124, wherein said biological sample comprises a body fluid (e.g., blood (e.g., whole blood, plasma, serum), urine, saliva, bone marrow, spinal fluid, sputum, ascites, lymphatic fluid, pleural fluid, amniotic fluid, semen, vaginal fluid, sweat, stool, glandular secretions, ocular fluids, breast milk) or a solid tissue sample.

126. The system of claim 124, wherein said biological sample is a diagnostic sample.

127. The system of any one of claims 72-126, wherein said genomic region of interest is a genetic locus comprising CYP2D6, CYP2D7, and CYP2D8.

128. The system of claim 127, wherein said analyzing comprises identifying one or more genetic variations in CYP2D6.

129. The system of claim 128, wherein said output comprises an identification of a subject as having a reduction, a loss of, or an increase in CYP2D6 function based on said genetic variation.

130. The system of claim 129, wherein said output comprises a recommendation of a treatment or an alternative treatment to said subject based on said identification.

131. The system of claim 129, wherein, when said subject is identified as having a reduction in, a loss of, or an increase in CYP2D6 function, said output further comprises a recommendation of an alternative treatment to said subject.

132. The system of claim 129, wherein said output further provides a recommendation of a dosage of a therapeutic to said subject based on said identification.

133. The system of claim 129, wherein, when said subject is identified as having a reduction in, a loss of, or an increase in CYP2D6 function, said output further comprises a recommendation to alter a dosage of a therapeutic.

134. The system of any one of claims 72-133, wherein said outer pair of gRNAs, said inner pair of gRNAs, or both, comprise gRNAs selected from any one of SEQ ID NOS: 1-418.


SEQUENCE LISTING (418 sequences)

SEQ ID NO: 1 - RNA, 100 nt, Artificial Sequence
  Description of Artificial Sequence Synthetic polynucleotide
aaggugguggacacucgugaguuuuagagcuagaaauagcaaguuaaaauaaggcuaguccguuaucaacuugaaaaaguggcaccgagucggugcuuuu

SEQ ID NO: 2 - RNA, 100 nt, Artificial Sequence
  Description of Artificial Sequence Synthetic polynucleotide
cacuauggagauuguguccaguuuuagagcuagaaauagcaaguuaaaauaaggcuaguccguuaucaacuugaaaaaguggcaccgagucggugcuuuu

SEQ ID NO: 3 - RNA, 100 nt, Artificial Sequence
  Description of Artificial Sequence Synthetic polynucleotide
acggacacuaccaaggagcgguuuuagagcuagaaauagcaaguuaaaauaaggcuaguccguuaucaacuugaaaaaguggcaccgagucggugcuuuu

SEQ ID NO: 4 - RNA, 100 nt, Artificial Sequence
  Description of Artificial Sequence Synthetic polynucleotide
cuugaagaaccuccucguggguuuuagagcuagaaauagcaaguuaaaauaaggcuaguccguuaucaacuugaaaaaguggcaccgagucggugcuuuu

SEQ ID NO: 5 - RNA, 100 nt, Artificial Sequence
  Description of Artificial Sequence Synthetic polynucleotide
augucucaagacuaccccucguuuuagagcuagaaauagcaaguuaaaauaaggcuaguccguuaucaacuugaaaaaguggcaccgagucggugcuuuu

SEQ ID NO: 6 - RNA, 100 nt, Artificial Sequence
  Description of Artificial Sequence Synthetic polynucleotide
cugucaugggcacguagaccguuuuagagcuagaaauagcaaguuaaaauaaggcuaguccguuaucaacuugaaaaaguggcaccgagucggugcuuuu

SEQ ID NO: 7 - RNA, 100 nt, Artificial Sequence
  Description of Artificial Sequence Synthetic polynucleotide
uccucaccgacauaaugggcguuuuagagcuagaaauagcaaguuaaaauaaggcuaguccguuaucaacuugaaaaaguggcaccgagucggugcuuuu

SEQ ID NO: 8 - RNA, 100 nt, Artificial Sequence
  Description of Artificial Sequence Synthetic polynucleotide
ggcuuacaaguugguccuaaguuuuagagcuagaaauagcaaguuaaaauaaggcuaguccguuaucaacuugaaaaaguggcaccgagucggugcuuuu

SEQ ID NO: 9 - RNA, 100 nt, Artificial Sequence
  Description of Artificial Sequence Synthetic polynucleotide
uaucaccuuuuagucaauucguuuuagagcuagaaauagcaaguuaaaauaaggcuaguccguuaucaacuugaaaaaguggcaccgagucggugcuuuu

SEQ ID NO: 10 - RNA, 100 nt, Artificial Sequence
  Description of Artificial Sequence Synthetic polynucleotide
ugucaagaauuaguggugguguuuuagagcuagaaauagcaaguuaaaauaaggcuaguccguuaucaacuugaaaaaguggcaccgagucggugcuuuu

SEQ ID NO: 11 - RNA, 100 nt, Artificial Sequence
  Description of Artificial Sequence Synthetic polynucleotide
ccauucacccuuaugcucagguuuuagagcuagaaauagcaaguuaaaauaaggcuaguccguuaucaacuugaaaaaguggcaccgagucggugcuuuu

SEQ ID NO: 12 - RNA, 100 nt, Artificial Sequence
  Description of Artificial Sequence Synthetic polynucleotide
aaccuccgguugcuuccugaguuuuagagcuagaaauagcaaguuaaaauaaggcuaguccguuaucaacuugaaaaaguggcaccgagucggugcuuuu

SEQ ID NO: 13 - RNA, 100 nt, Artificial Sequence
  Description of Artificial Sequence Synthetic polynucleotide
gguggacacucgugauggaaguuuuagagcuagaaauagcaaguuaaaauaaggcuaguccguuaucaacuugaaaaaguggcaccgagucggugcuuuu

SEQ ID NO: 14 - RNA, 36 nt, Artificial Sequence
  Description of Artificial Sequence Synthetic oligonucleotide
gguggacacucgugauggaaguuuuagagcuaugcu

SEQ ID NO: 15 - RNA, 36 nt, Artificial Sequence
  Description of Artificial Sequence Synthetic oligonucleotide
aaggugguggacacucgugaguuuuagagcuaugcu

SEQ ID NO: 16 - RNA, 36 nt, Artificial Sequence
  Description of Artificial Sequence Synthetic oligonucleotide
cacuauggagauuguguccaguuuuagagcuaugcu

SEQ ID NO: 17 - RNA, 36 nt, Artificial Sequence
  Description of Artificial Sequence Synthetic oligonucleotide
acggacacuaccaaggagcgguuuuagagcuaugcu

SEQ ID NO: 18 - RNA, 36 nt, Artificial Sequence
  Description of Artificial Sequence Synthetic oligonucleotide
cuugaagaaccuccucguggguuuuagagcuaugcu

SEQ ID NO: 19 - RNA, 36 nt, Artificial Sequence
  Description of Artificial Sequence Synthetic oligonucleotide
augucucaagacuaccccucguuuuagagcuaugcu

SEQ ID NO: 20 - RNA, 36 nt, Artificial Sequence
  Description of Artificial Sequence Synthetic oligonucleotide
cugucaugggcacguagaccguuuuagagcuaugcu

SEQ ID NO: 21 - RNA, 36 nt, Artificial Sequence
  Description of Artificial Sequence Synthetic oligonucleotide
uccucaccgacauaaugggcguuuuagagcuaugcu

SEQ ID NO: 22 - RNA, 36 nt, Artificial Sequence
  Description of Artificial Sequence Synthetic oligonucleotide
ggcuuacaaguugguccuaaguuuuagagcuaugcu

SEQ ID NO: 23 - RNA, 36 nt, Artificial Sequence
  Description of Artificial Sequence Synthetic oligonucleotide
uaucaccuuuuagucaauucguuuuagagcuaugcu

SEQ ID NO: 24 - RNA, 36 nt, Artificial Sequence
  Description of Artificial Sequence Synthetic oligonucleotide
ugucaagaauuaguggugguguuuuagagcuaugcu

SEQ ID NO: 25 - RNA, 36 nt, Artificial Sequence
  Description of Artificial Sequence Synthetic oligonucleotide
ccauucacccuuaugcucagguuuuagagcuaugcu

SEQ ID NO: 26 - RNA, 36 nt, Artificial Sequence
  Description of Artificial Sequence Synthetic oligonucleotide
aaccuccgguugcuuccugaguuuuagagcuaugcu

SEQ ID NO: 27 - RNA, 20 nt, Artificial Sequence
  Description of Artificial Sequence Synthetic oligonucleotide
ugguccauguuuucaagagu

SEQ ID NO: 28 - RNA, 20 nt, Artificial Sequence
  Description of Artificial Sequence Synthetic oligonucleotide
acucaaaccagugacaccac

SEQ ID NO: 29 - RNA, 20 nt, Artificial Sequence
  Description of Artificial Sequence Synthetic oligonucleotide
aaagacccaagacguuggaa

SEQ ID NO: 30 - RNA, 20 nt, Artificial Sequence
  Description of Artificial Sequence Synthetic oligonucleotide
guucagaaaacacuagaccc

SEQ ID NO: 31 - RNA, 20 nt, Artificial Sequence
  Description of Artificial Sequence Synthetic oligonucleotide
gggucuaguguuuucugaac

SEQ ID NO: 32 - RNA, 20 nt, Artificial Sequence
  Description of Artificial Sequence Synthetic oligonucleotide
acccucaucucaugaaggac

SEQ ID NO: 33 - RNA, 20 nt, Artificial Sequence
  Description of Artificial Sequence Synthetic oligonucleotide
acuugucaucggaacaaauu

SEQ ID NO: 34 - RNA, 20 nt, Artificial Sequence
  Description of Artificial Sequence Synthetic oligonucleotide
cuccccccacauugucacua

SEQ ID NO: 35 - RNA, 20 nt, Artificial Sequence
  Description of Artificial Sequence Synthetic oligonucleotide
ccagggguaccacggaacag

SEQ ID NO: 36 - RNA, 20 nt, Artificial Sequence
  Description of Artificial Sequence Synthetic oligonucleotide
cccucaucucaugaaggacg

SEQ ID NO: 37 - RNA, 20 nt, Artificial Sequence
  Description of Artificial Sequence Synthetic oligonucleotide
acacacccgagaccaaugcc

SEQ ID NO: 38 - RNA, 20 nt, Artificial Sequence
  Description of Artificial Sequence Synthetic oligonucleotide
aacagccauuccaacgucuu

SEQ ID NO: 39 - RNA, 20 nt, Artificial Sequence
  Description of Artificial Sequence Synthetic oligonucleotide
uaccacggaacagcggcugu

SEQ ID NO: 40 - RNA, 35 nt, Artificial Sequence
  Description of Artificial Sequence Synthetic oligonucleotide
ugguccauguuuucaagaguguuuagagcuaugcu

SEQ ID NO: 41 - RNA, 35 nt, Artificial Sequence
  Description of Artificial Sequence Synthetic oligonucleotide
acucaaaccagugacaccacguuuagagcuaugcu

SEQ ID NO: 42 - RNA, 35 nt, Artificial Sequence
  Description of Artificial Sequence Synthetic oligonucleotide
aaagacccaagacguuggaaguuuagagcuaugcu

SEQ ID NO: 43 - RNA, 35 nt, Artificial Sequence
  Description of Artificial Sequence Synthetic oligonucleotide
guucagaaaacacuagacccguuuagagcuaugcu

SEQ ID NO: 44 - RNA, 35 nt, Artificial Sequence
  Description of Artificial Sequence Synthetic oligonucleotide
gggucuaguguuuucugaacguuuagagcuaugcu

SEQ ID NO: 45 - RNA, 35 nt, Artificial Sequence
  Description of Artificial Sequence Synthetic oligonucleotide
acccucaucucaugaaggacguuuagagcuaugcu

SEQ ID NO: 46 - RNA, 35 nt, Artificial Sequence
  Description of Artificial Sequence Synthetic oligonucleotide
acuugucaucggaacaaauuguuuagagcuaugcu

SEQ ID NO: 47 - RNA, 35 nt, Artificial Sequence
  Description of Artificial Sequence Synthetic oligonucleotide
cuccccccacauugucacuaguuuagagcuaugcu

SEQ ID NO: 48 - RNA, 35 nt, Artificial Sequence
  Description of Artificial Sequence Synthetic oligonucleotide
ccagggguaccacggaacagguuuagagcuaugcu

SEQ ID NO: 49 - RNA, 35 nt, Artificial Sequence
  Description of Artificial Sequence Synthetic oligonucleotide
cccucaucucaugaaggacgguuuagagcuaugcu

SEQ ID NO: 50 - RNA, 35 nt, Artificial Sequence
  Description of Artificial Sequence Synthetic oligonucleotide
acacacccgagaccaaugccguuuagagcuaugcu

SEQ ID NO: 51 - RNA, 35 nt, Artificial Sequence
  Description of Artificial Sequence Synthetic oligonucleotide
aacagccauuccaacgucuuguuuagagcuaugcu

SEQ ID NO: 52 - RNA, 35 nt, Artificial Sequence
  Description of Artificial Sequence Synthetic oligonucleotide
uaccacggaacagcggcuguguuuagagcuaugcu

SEQ ID NO: 53 - RNA, 100 nt, Artificial Sequence
  Description of Artificial Sequence Synthetic polynucleotide
ugguccauguuuucaagaguguuuuagagcuagaaauagcaaguuaaaauaaggcuaguccguuaucaacuugaaaaaguggcaccgagucggugcuuuu

SEQ ID NO: 54 - RNA, 100 nt, Artificial Sequence
  Description of Artificial Sequence Synthetic polynucleotide
acucaaaccagugacaccacguuuuagagcuagaaauagcaaguuaaaauaaggcuaguccguuaucaacuugaaaaaguggcaccgagucggugcuuuu

SEQ ID NO: 55 - RNA, 100 nt, Artificial Sequence
  Description of Artificial Sequence Synthetic polynucleotide
aaagacccaagacguuggaaguuuuagagcuagaaauagcaaguuaaaauaaggcuaguccguuaucaacuugaaaaaguggcaccgagucggugcuuuu

SEQ ID NO: 56 - RNA, 100 nt, Artificial Sequence
  Description of Artificial Sequence Synthetic polynucleotide
guucagaaaacacuagacccguuuuagagcuagaaauagcaaguuaaaauaaggcuaguccguuaucaacuugaaaaaguggcaccgagucggugcuuuu

SEQ ID NO: 57 - RNA, 100 nt, Artificial Sequence
  Description of Artificial Sequence Synthetic polynucleotide
gggucuaguguuuucugaacguuuuagagcuagaaauagcaaguuaaaauaaggcuaguccguuaucaacuugaaaaaguggcaccgagucggugcuuuu

SEQ ID NO: 58 - RNA, 100 nt, Artificial Sequence
  Description of Artificial Sequence Synthetic polynucleotide
acccucaucucaugaaggacguuuuagagcuagaaauagcaaguuaaaauaaggcuaguccguuaucaacuugaaaaaguggcaccgagucggugcuuuu

SEQ ID NO: 59 - RNA, 100 nt, Artificial Sequence
  Description of Artificial Sequence Synthetic polynucleotide
acuugucaucggaacaaauuguuuuagagcuagaaauagcaaguuaaaauaaggcuaguccguuaucaacuugaaaaaguggcaccgagucggugcuuuu

SEQ ID NO: 60 - RNA, 100 nt, Artificial Sequence
  Description of Artificial Sequence Synthetic polynucleotide
cuccccccacauugucacuaguuuuagagcuagaaauagcaaguuaaaauaaggcuaguccguuaucaacuugaaaaaguggcaccgagucggugcuuuu

SEQ ID NO: 61 - RNA, 100 nt, Artificial Sequence
  Description of Artificial Sequence Synthetic polynucleotide
ccagggguaccacggaacagguuuuagagcuagaaauagcaaguuaaaauaaggcuaguccguuaucaacuugaaaaaguggcaccgagucggugcuuuu

SEQ ID NO: 62 - RNA, 100 nt, Artificial Sequence
  Description of Artificial Sequence Synthetic polynucleotide
cccucaucucaugaaggacgguuuuagagcuagaaauagcaaguuaaaauaaggcuaguccguuaucaacuugaaaaaguggcaccgagucggugcuuuu

SEQ ID NO: 63 - RNA, 100 nt, Artificial Sequence
  Description of Artificial Sequence Synthetic polynucleotide
acacacccgagaccaaugccguuuuagagcuagaaauagcaaguuaaaauaaggcuaguccguuaucaacuugaaaaaguggcaccgagucggugcuuuu

SEQ ID NO: 64 - RNA, 100 nt, Artificial Sequence
  Description of Artificial Sequence Synthetic polynucleotide
aacagccauuccaacgucuuguuuuagagcuagaaauagcaaguuaaaauaaggcuaguccguuaucaacuugaaaaaguggcaccgagucggugcuuuu

SEQ ID NO: 65 - RNA, 100 nt, Artificial Sequence
  Description of Artificial Sequence Synthetic polynucleotide
uaccacggaacagcggcuguguuuuagagcuagaaauagcaaguuaaaauaaggcuaguccguuaucaacuugaaaaaguggcaccgagucggugcuuuu

SEQ ID NO: 66 - RNA, 20 nt, Artificial Sequence
  Description of Artificial Sequence Synthetic oligonucleotide
aaggugguggacacucguga

SEQ ID NO: 67 - RNA, 20 nt, Artificial Sequence
  Description of Artificial Sequence Synthetic oligonucleotide
cacuauggagauugugucca

SEQ ID NO: 68 - RNA, 20 nt, Artificial Sequence
  Description of Artificial Sequence Synthetic oligonucleotide
acggacacuaccaaggagcg

SEQ ID NO: 69 - RNA, 20 nt, Artificial Sequence
  Description of Artificial Sequence Synthetic oligonucleotide
cuugaagaaccuccucgugg

SEQ ID NO: 70 - RNA, 20 nt, Artificial Sequence
  Description of Artificial Sequence Synthetic oligonucleotide
augucucaagacuaccccuc

SEQ ID NO: 71 - RNA, 20 nt, Artificial Sequence
  Description of Artificial Sequence Synthetic oligonucleotide
cugucaugggcacguagacc

SEQ ID NO: 72 - RNA, 20 nt, Artificial Sequence
  Description of Artificial Sequence Synthetic oligonucleotide
uccucaccgacauaaugggc

SEQ ID NO: 73 - RNA, 20 nt, Artificial Sequence
  Description of Artificial Sequence Synthetic oligonucleotide
ggcuuacaaguugguccuaa

SEQ ID NO: 74 - RNA, 20 nt, Artificial Sequence
  Description of Artificial Sequence Synthetic oligonucleotide
uaucaccuuuuagucaauuc

SEQ ID NO: 75 - RNA, 20 nt, Artificial Sequence
  Description of Artificial Sequence Synthetic oligonucleotide
ugucaagaauuagugguggu

SEQ ID NO: 76 - RNA, 19 nt, Artificial Sequence
  Description of Artificial Sequence Synthetic oligonucleotide
cauucacccuuaugcucag

SEQ ID NO: 77 - RNA, 20 nt, Artificial Sequence
  Description of Artificial Sequence Synthetic oligonucleotide
aaccuccgguugcuuccuga

SEQ ID NO: 78 - RNA, 20 nt, Artificial Sequence
  Description of Artificial Sequence Synthetic oligonucleotide
gguggacacucgugauggaa

SEQ ID NO: 79 - RNA, 20 nt, Artificial Sequence
  Description of Artificial Sequence Synthetic oligonucleotide
gguggacacucgugauggaa

SEQ ID NO: 80 - RNA, 20 nt, Artificial Sequence
  Description of Artificial Sequence Synthetic oligonucleotide
aaggugguggacacucguga

SEQ ID NO: 81 - RNA, 20 nt, Artificial Sequence
  Description of Artificial Sequence Synthetic oligonucleotide
cacuauggagauugugucca

SEQ ID NO: 82 - RNA, 20 nt, Artificial Sequence
  Description of Artificial Sequence Synthetic oligonucleotide
acggacacuaccaaggagcg

SEQ ID NO: 83 - RNA, 20 nt, Artificial Sequence
  Description of Artificial Sequence Synthetic oligonucleotide
cuugaagaaccuccucgugg

SEQ ID NO: 84 - RNA, 20 nt, Artificial Sequence
  Description of Artificial Sequence Synthetic oligonucleotide
augucucaagacuaccccuc

SEQ ID NO: 85 - RNA, 20 nt, Artificial Sequence
  Description of Artificial Sequence Synthetic oligonucleotide
cugucaugggcacguagacc

SEQ ID NO: 86 - RNA, 20 nt, Artificial Sequence
  Description of Artificial Sequence Synthetic oligonucleotide
uccucaccgacauaaugggc

SEQ ID NO: 87 - RNA, 20 nt, Artificial Sequence
  Description of Artificial Sequence Synthetic oligonucleotide
ggcuuacaaguugguccuaa

SEQ ID NO: 88 - RNA, 20 nt, Artificial Sequence
  Description of Artificial Sequence Synthetic oligonucleotide
uaucaccuuuuagucaauuc

SEQ ID NO: 89 - RNA, 20 nt, Artificial Sequence
  Description of Artificial Sequence Synthetic oligonucleotide
ugucaagaauuagugguggu

SEQ ID NO: 90 - RNA, 20 nt, Artificial Sequence
  Description of Artificial Sequence Synthetic oligonucleotide
ccauucacccuuaugcucag

SEQ ID NO: 91 - RNA, 20 nt, Artificial Sequence
  Description of Artificial Sequence Synthetic oligonucleotide
aaccuccgguugcuuccuga

SEQ ID NO: 92 - RNA, 20 nt, Artificial Sequence
  Description of Artificial Sequence Synthetic oligonucleotide
gaggucaccaacuugggcag

SEQ ID NO: 93 - RNA, 20 nt, Artificial Sequence
  Description of Artificial Sequence Synthetic oligonucleotide
cccaaguuggugaccucagc

SEQ ID NO: 94 - RNA, 20 nt, Artificial Sequence
  Description of Artificial Sequence Synthetic oligonucleotide
ccagcugaggucaccaacuu

SEQ ID NO: 95 - RNA, 20 nt, Artificial Sequence
  Description of Artificial Sequence Synthetic oligonucleotide
aggugccgaacacuggugag

SEQ ID NO: 96 - RNA, 20 nt, Artificial Sequence
  Description of Artificial Sequence Synthetic oligonucleotide
ggaccccgagguaacugcug

SEQ ID NO: 97 - RNA, 20 nt, Artificial Sequence
  Description of Artificial Sequence Synthetic oligonucleotide
ggccuugaagaaccuccucg

SEQ ID NO: 98 - RNA, 20 nt, Artificial Sequence
  Description of Artificial Sequence Synthetic oligonucleotide
ugacucugaggcucucggau

SEQ ID NO: 99 - RNA, 20 nt, Artificial Sequence
  Description of Artificial Sequence Synthetic oligonucleotide
ucgugaagcccauuuucagu

SEQ ID NO: 100 - RNA, 20 nt, Artificial Sequence
  Description of Artificial Sequence Synthetic oligonucleotide
acugaaaaugggcuucacga

SEQ ID NO: 101 - RNA, 20 nt, Artificial Sequence
  Description of Artificial Sequence Synthetic oligonucleotide
cacgacccagcgaccuccug

SEQ ID NO: 102 - RNA, 20 nt, Artificial Sequence
  Description of Artificial Sequence Synthetic oligonucleotide
gaugcuuuggcaagauggcg

SEQ ID NO: 103 - RNA, 20 nt, Artificial Sequence
  Description of Artificial Sequence Synthetic oligonucleotide
uugaagaaccuccucguggc

SEQ ID NO: 104 - RNA, 20 nt, Artificial Sequence
  Description of Artificial Sequence Synthetic oligonucleotide
acaugaacgaggccaagcgg

SEQ ID NO: 105 - RNA, 20 nt, Artificial Sequence
  Description of Artificial Sequence Synthetic oligonucleotide
caugaacgaggccaagcgga

SEQ ID NO: 106 - RNA, 20 nt, Artificial Sequence
  Description of Artificial Sequence Synthetic oligonucleotide
cgacagaugguguaguccaa

SEQ ID NO: 107 - RNA, 20 nt, Artificial Sequence
  Description of Artificial Sequence Synthetic oligonucleotide
cuugaagaaccuccucgugg

SEQ ID NO: 108 - RNA, 20 nt, Artificial Sequence
  Description of Artificial Sequence Synthetic oligonucleotide
aaugggcuucacgaaggugc

SEQ ID NO: 109 - RNA, 20 nt, Artificial Sequence
  Description of Artificial Sequence Synthetic oligonucleotide
gaaugucccugucuacgaug

SEQ ID NO: 110 - RNA, 20 nt, Artificial Sequence
  Description of Artificial Sequence Synthetic oligonucleotide
agggucacccgagccuacca

SEQ ID NO: 111 - RNA, 20 nt, Artificial Sequence
  Description of Artificial Sequence Synthetic oligonucleotide
acggacacuaccaaggagcg

SEQ ID NO: 112 - RNA, 20 nt, Artificial Sequence
  Description of Artificial Sequence Synthetic oligonucleotide
gacacuaccaaggagcgcgg

SEQ ID NO: 113 - RNA, 20 nt, Artificial Sequence
  Description of Artificial Sequence Synthetic oligonucleotide
uuucagucgggacaugaacg

SEQ ID NO: 114 - RNA, 20 nt, Artificial Sequence
  Description of Artificial Sequence Synthetic oligonucleotide
acacuaccaaggagcgcggc

SEQ ID NO: 115 - RNA, 20 nt, Artificial Sequence
  Description of Artificial Sequence Synthetic oligonucleotide
gggucacccgagccuaccau

SEQ ID NO: 116 - RNA, 20 nt, Artificial Sequence
  Description of Artificial Sequence Synthetic oligonucleotide
ugagagguagcggcuuacgu

SEQ ID NO: 117 - RNA, 35 nt, Artificial Sequence
  Description of Artificial Sequence Synthetic oligonucleotide
gaggucaccaacuugggcagguuuagagcuaugcu

SEQ ID NO: 118 - RNA, 35 nt, Artificial Sequence
  Description of Artificial Sequence Synthetic oligonucleotide
cccaaguuggugaccucagcguuuagagcuaugcu

SEQ ID NO: 119 - RNA, 35 nt, Artificial Sequence
  Description of Artificial Sequence Synthetic oligonucleotide
ccagcugaggucaccaacuuguuuagagcuaugcu

SEQ ID NO: 120 - RNA, 35 nt, Artificial Sequence
  Description of Artificial Sequence Synthetic oligonucleotide
aggugccgaacacuggugagguuuagagcuaugcu

SEQ ID NO: 121 - RNA, 35 nt, Artificial Sequence
  Description of Artificial Sequence Synthetic oligonucleotide
ggaccccgagguaacugcugguuuagagcuaugcu

SEQ ID NO: 122 - RNA, 35 nt, Artificial Sequence
  Description of Artificial Sequence Synthetic oligonucleotide
ggccuugaagaaccuccucgguuuagagcuaugcu

SEQ ID NO: 123 - RNA, 35 nt, Artificial Sequence
  Description of Artificial Sequence Synthetic oligonucleotide
ugacucugaggcucucggauguuuagagcuaugcu

SEQ ID NO: 124 - RNA, 35 nt, Artificial Sequence
  Description of Artificial Sequence Synthetic oligonucleotide
ucgugaagcccauuuucaguguuuagagcuaugcu

SEQ ID NO: 125 - RNA, 35 nt, Artificial Sequence
  Description of Artificial Sequence Synthetic oligonucleotide
acugaaaaugggcuucacgaguuuagagcuaugcu

SEQ ID NO: 126 - RNA, 35 nt, Artificial Sequence
  Description of Artificial Sequence Synthetic oligonucleotide
cacgacccagcgaccuccugguuuagagcuaugcu

SEQ ID NO: 127 - RNA, 35 nt, Artificial Sequence
  Description of Artificial Sequence Synthetic oligonucleotide
gaugcuuuggcaagauggcgguuuagagcuaugcu

SEQ ID NO: 128 - RNA, 35 nt, Artificial Sequence
  Description of Artificial Sequence Synthetic oligonucleotide
uugaagaaccuccucguggcguuuagagcuaugcu

SEQ ID NO: 129 - RNA, 35 nt, Artificial Sequence
  Description of Artificial Sequence Synthetic oligonucleotide
acaugaacgaggccaagcggguuuagagcuaugcu

SEQ ID NO: 130 - RNA, 35 nt, Artificial Sequence
  Description of Artificial Sequence Synthetic oligonucleotide
caugaacgaggccaagcggaguuuagagcuaugcu

SEQ ID NO: 131 - RNA, 35 nt, Artificial Sequence
  Description of Artificial Sequence Synthetic oligonucleotide
cgacagaugguguaguccaaguuuagagcuaugcu

SEQ ID NO: 132 - RNA, 35 nt, Artificial Sequence
  Description of Artificial Sequence Synthetic oligonucleotide
cuugaagaaccuccucguggguuuagagcuaugcu

SEQ ID NO: 133 - RNA, 35 nt, Artificial Sequence
  Description of Artificial Sequence Synthetic oligonucleotide
aaugggcuucacgaaggugcguuuagagcuaugcu

SEQ ID NO: 134 - RNA, 35 nt, Artificial Sequence
  Description of Artificial Sequence Synthetic oligonucleotide
gaaugucccugucuacgaugguuuagagcuaugcu

SEQ ID NO: 135 - RNA, 35 nt, Artificial Sequence
  Description of Artificial Sequence Synthetic oligonucleotide
agggucacccgagccuaccaguuuagagcuaugcu

SEQ ID NO: 136 - RNA, 35 nt, Artificial Sequence
  Description of Artificial Sequence Synthetic oligonucleotide
acggacacuaccaaggagcgguuuagagcuaugcu

SEQ ID NO: 137 - RNA, 35 nt, Artificial Sequence
  Description of Artificial Sequence Synthetic oligonucleotide
gacacuaccaaggagcgcggguuuagagcuaugcu

SEQ ID NO: 138 - RNA, 35 nt, Artificial Sequence
  Description of Artificial Sequence Synthetic oligonucleotide
uuucagucgggacaugaacgguuuagagcuaugcu

SEQ ID NO: 139 - RNA, 35 nt, Artificial Sequence
  Description of Artificial Sequence Synthetic oligonucleotide
acacuaccaaggagcgcggcguuuagagcuaugcu

SEQ ID NO: 140 - RNA, 35 nt, Artificial Sequence
  Description of Artificial Sequence Synthetic oligonucleotide
gggucacccgagccuaccauguuuagagcuaugcu

SEQ ID NO: 141 - RNA, 35 nt, Artificial Sequence
  Description of Artificial Sequence Synthetic oligonucleotide
ugagagguagcggcuuacguguuuagagcuaugcu

SEQ ID NO: 142 - RNA, 100 nt, Artificial Sequence
  Description of Artificial Sequence Synthetic polynucleotide
gaggucaccaacuugggcagguuuuagagcuagaaauagcaaguuaaaauaaggcuaguccguuaucaacuugaaaaaguggcaccgagucggugcuuuu

SEQ ID NO: 143 - RNA, 100 nt, Artificial Sequence
  Description of Artificial Sequence Synthetic polynucleotide
cccaaguuggugaccucagcguuuuagagcuagaaauagcaaguuaaaauaaggcuaguccguuaucaacuugaaaaaguggcaccgagucggugcuuuu

SEQ ID NO: 144 - RNA, 100 nt, Artificial Sequence
  Description of Artificial Sequence Synthetic polynucleotide
ccagcugaggucaccaacuuguuuuagagcuagaaauagcaaguuaaaauaaggcuaguccguuaucaacuugaaaaaguggcaccgagucggugcuuuu

SEQ ID NO: 145 - RNA, 100 nt, Artificial Sequence
  Description of Artificial Sequence Synthetic polynucleotide
aggugccgaacacuggugagguuuuagagcuagaaauagcaaguuaaaauaaggcuaguccguuaucaacuugaaaaaguggcaccgagucggugcuuuu

SEQ ID NO: 146 - RNA, 100 nt, Artificial Sequence
  Description of Artificial Sequence Synthetic polynucleotide
ggaccccgagguaacugcugguuuuagagcuagaaauagcaaguuaaaauaaggcuaguccguuaucaacuugaaaaaguggcaccgagucggugcuuuu

SEQ ID NO: 147 - RNA, 100 nt, Artificial Sequence
  Description of Artificial Sequence Synthetic polynucleotide
ggccuugaagaaccuccucgguuuuagagcuagaaauagcaaguuaaaauaaggcuaguccguuaucaacuugaaaaaguggcaccgagucggugcuuuu

SEQ ID NO: 148 - RNA, 100 nt, Artificial Sequence
  Description of Artificial Sequence Synthetic polynucleotide
ugacucugaggcucucggauguuuuagagcuagaaauagcaaguuaaaauaaggcuaguccguuaucaacuugaaaaaguggcaccgagucggugcuuuu

SEQ ID NO: 149 - RNA, 100 nt, Artificial Sequence
  Description of Artificial Sequence Synthetic polynucleotide
ucgugaagcccauuuucaguguuuuagagcuagaaauagcaaguuaaaauaaggcuaguccguuaucaacuugaaaaaguggcaccgagucggugcuuuu

SEQ ID NO: 150 - RNA, 100 nt, Artificial Sequence
  Description of Artificial Sequence Synthetic polynucleotide
acugaaaaugggcuucacgaguuuuagagcuagaaauagcaaguuaaaauaaggcuaguccguuaucaacuugaaaaaguggcaccgagucggugcuuuu

SEQ ID NO: 151 - RNA, 100 nt, Artificial Sequence
  Description of Artificial Sequence Synthetic polynucleotide
cacgacccagcgaccuccugguuuuagagcuagaaauagcaaguuaaaauaaggcuaguccguuaucaacuugaaaaaguggcaccgagucggugcuuuu

SEQ ID NO: 152 - RNA, 100 nt, Artificial Sequence
  Description of Artificial Sequence Synthetic polynucleotide
gaugcuuuggcaagauggcgguuuuagagcuagaaauagcaaguuaaaauaaggcuaguccguuaucaacuugaaaaaguggcaccgagucggugcuuuu

SEQ ID NO: 153 - RNA, 100 nt, Artificial Sequence
  Description of Artificial Sequence Synthetic polynucleotide
uugaagaaccuccucguggcguuuuagagcuagaaauagcaaguuaaaauaaggcuaguccguuaucaacuugaaaaaguggcaccgagucggugcuuuu

SEQ ID NO: 154 - RNA, 100 nt, Artificial Sequence
  Description of Artificial Sequence Synthetic polynucleotide
acaugaacgaggccaagcggguuuuagagcuagaaauagcaaguuaaaauaaggcuaguccguuaucaacuugaaaaaguggcaccgagucggugcuuuu

SEQ ID NO: 155 - RNA, 100 nt, Artificial Sequence
  Description of Artificial Sequence Synthetic polynucleotide
caugaacgaggccaagcggaguuuuagagcuagaaauagcaaguuaaaauaaggcuaguccguuaucaacuugaaaaaguggcaccgagucggugcuuuu

SEQ ID NO: 156 - RNA, 100 nt, Artificial Sequence
  Description of Artificial Sequence Synthetic polynucleotide
cgacagaugguguaguccaaguuuuagagcuagaaauagcaaguuaaaauaaggcuaguccguuaucaacuugaaaaaguggcaccgagucggugcuuuu

SEQ ID NO: 157 - RNA, 100 nt, Artificial Sequence; identical to SEQ ID NO: 4
  Description of Artificial Sequence Synthetic polynucleotide

SEQ ID NO: 158 - RNA, 100 nt, Artificial Sequence
  Description of Artificial Sequence Synthetic polynucleotide
aaugggcuucacgaaggugcguuuuagagcuagaaauagcaaguuaaaauaaggcuaguccguuaucaacuugaaaaaguggcaccgagucggugcuuuu

SEQ ID NO: 159 - RNA, 100 nt, Artificial Sequence
  Description of Artificial Sequence Synthetic polynucleotide
gaaugucccugucuacgaugguuuuagagcuagaaauagcaaguuaaaauaaggcuaguccguuaucaacuugaaaaaguggcaccgagucggugcuuuu

SEQ ID NO: 160 - RNA, 100 nt, Artificial Sequence
  Description of Artificial Sequence Synthetic polynucleotide
agggucacccgagccuaccaguuuuagagcuagaaauagcaaguuaaaauaaggcuaguccguuaucaacuugaaaaaguggcaccgagucggugcuuuu

SEQ ID NO: 161 - RNA, 100 nt, Artificial Sequence; identical to SEQ ID NO: 3
  Description of Artificial Sequence Synthetic polynucleotide

SEQ ID NO: 162 - RNA, 100 nt, Artificial Sequence
  Description of Artificial Sequence Synthetic polynucleotide
gacacuaccaaggagcgcggguuuuagagcuagaaauagcaaguuaaaauaaggcuaguccguuaucaacuugaaaaaguggcaccgagucggugcuuuu

SEQ ID NO: 163 - RNA, 100 nt, Artificial Sequence
  Description of Artificial Sequence Synthetic polynucleotide
uuucagucgggacaugaacgguuuuagagcuagaaauagcaaguuaaaauaaggcuaguccguuaucaacuugaaaaaguggcaccgagucggugcuuuu

SEQ ID NO: 164 - RNA, 100 nt, Artificial Sequence
  Description of Artificial Sequence Synthetic polynucleotide
acacuaccaaggagcgcggcguuuuagagcuagaaauagcaaguuaaaauaaggcuaguccguuaucaacuugaaaaaguggcaccgagucggugcuuuu

SEQ ID NO: 165 - RNA, 100 nt, Artificial Sequence
  Description of Artificial Sequence Synthetic polynucleotide
gggucacccgagccuaccauguuuuagagcuagaaauagcaaguuaaaauaaggcuaguccguuaucaacuugaaaaaguggcaccgagucggugcuuuu

SEQ ID NO: 166 - RNA, 100 nt, Artificial Sequence
  Description of Artificial Sequence Synthetic polynucleotide
ugagagguagcggcuuacguguuuuagagcuagaaauagcaaguuaaaauaaggcuaguccguuaucaacuugaaaaaguggcaccgagucggugcuuuu

SEQ ID NO: 167 - RNA, 20 nt, Artificial Sequence
  Description of Artificial Sequence Synthetic oligonucleotide
uuaaugcuagaauuaggcac

SEQ ID NO: 168 - RNA, 20 nt, Artificial Sequence
  Description of Artificial Sequence Synthetic oligonucleotide
uuaggcacaggcuuacaagu

SEQ ID NO: 169 - RNA, 20 nt, Artificial Sequence
  Description of Artificial Sequence Synthetic oligonucleotide
gaaguggccugcccuucaaa

SEQ ID NO: 170 - RNA, 20 nt, Artificial Sequence
  Description of Artificial Sequence Synthetic oligonucleotide
ggcuuacaaguugguccuaa

SEQ ID NO: 171 - RNA, 35 nt, Artificial Sequence
  Description of Artificial Sequence Synthetic oligonucleotide
uuaaugcuagaauuaggcacguuuagagcuaugcu

SEQ ID NO: 172 - RNA, 35 nt, Artificial Sequence
  Description of Artificial Sequence Synthetic oligonucleotide
uuaggcacaggcuuacaaguguuuagagcuaugcu

SEQ ID NO: 173 - RNA, 35 nt, Artificial Sequence
  Description of Artificial Sequence Synthetic oligonucleotide
gaaguggccugcccuucaaaguuuagagcuaugcu

SEQ ID NO: 174 - RNA, 35 nt, Artificial Sequence
  Description of Artificial Sequence Synthetic oligonucleotide
ggcuuacaaguugguccuaaguuuagagcuaugcu

SEQ ID NO: 175 - RNA, 100 nt, Artificial Sequence
  Description of Artificial Sequence Synthetic polynucleotide
uuaaugcuagaauuaggcacguuuuagagcuagaaauagcaaguuaaaauaaggcuaguccguuaucaacuugaaaaaguggcaccgagucggugcuuuu

SEQ ID NO: 176 - RNA, 100 nt, Artificial Sequence
  Description of Artificial Sequence Synthetic polynucleotide
uuaggcacaggcuuacaaguguuuuagagcuagaaauagcaaguuaaaauaaggcuaguccguuaucaacuugaaaaaguggcaccgagucggugcuuuu

SEQ ID NO: 177 - RNA, 100 nt, Artificial Sequence
  Description of Artificial Sequence Synthetic polynucleotide
gaaguggccugcccuucaaaguuuuagagcuagaaauagcaaguuaaaauaaggcuaguccguuaucaacuugaaaaaguggcaccgagucggugcuuuu

SEQ ID NO: 178 - RNA, 100 nt, Artificial Sequence; identical to SEQ ID NO: 8
  Description of Artificial Sequence Synthetic polynucleotide

SEQ ID NO: 179 - RNA, 20 nt, Artificial Sequence
  Description of Artificial Sequence Synthetic oligonucleotide
cuaaacaacaauuuagcugu

SEQ ID NO: 180 - RNA, 35 nt, Artificial Sequence
  Description of Artificial Sequence Synthetic oligonucleotide
cuaaacaacaauuuagcuguguuuagagcuaugcu

SEQ ID NO: 181 - RNA, 100 nt, Artificial Sequence
  Description of Artificial Sequence Synthetic polynucleotide
cuaaacaacaauuuagcuguguuuuagagcuagaaauagcaaguuaaaauaaggcuaguccguuaucaacuugaaaaaguggcaccgagucggugcuuuu

SEQ ID NO: 182 - RNA, 20 nt, Artificial Sequence
  Description of Artificial Sequence Synthetic oligonucleotide
cuucacgguucugagucuug

SEQ ID NO: 183 - RNA, 20 nt, Artificial Sequence
  Description of Artificial Sequence Synthetic oligonucleotide
accgagccgugugaccacag

SEQ ID NO: 184 - RNA, 20 nt, Artificial Sequence
  Description of Artificial Sequence Synthetic oligonucleotide
ucuguccucaccgacauaau

SEQ ID NO: 185 - RNA, 20 nt, Artificial Sequence
  Description of Artificial Sequence Synthetic oligonucleotide
aggugaagcagccuucucgu

SEQ ID NO: 186 - RNA, 20 nt, Artificial Sequence
  Description of Artificial Sequence Synthetic oligonucleotide
ucugacugacucggugccag

SEQ ID NO: 187 - RNA, 20 nt, Artificial Sequence
  Description of Artificial Sequence Synthetic oligonucleotide
uucugacugacucggugcca

SEQ ID NO: 188 - RNA, 20 nt, Artificial Sequence
  Description of Artificial Sequence Synthetic oligonucleotide
acuguggucacacggcucgg

SEQ ID NO: 189 - RNA, 20 nt, Artificial Sequence
  Description of Artificial Sequence Synthetic oligonucleotide
uucccuaagaaggucugccc

SEQ ID NO: 190 - RNA, 20 nt, Artificial Sequence
  Description of Artificial Sequence Synthetic oligonucleotide
gucuguccucaccgacauaa

SEQ ID NO: 191 - RNA, 20 nt, Artificial Sequence
  Description of Artificial Sequence Synthetic oligonucleotide
ccucaccgacauaaugggcu

SEQ ID NO: 192 - RNA, 20 nt, Artificial Sequence
  Description of Artificial Sequence Synthetic oligonucleotide
ggcacguagacccgguccca

SEQ ID NO: 193 - RNA, 35 nt, Artificial Sequence
  Description of Artificial Sequence Synthetic oligonucleotide
cuucacgguucugagucuugguuuagagcuaugcu

SEQ ID NO: 194 - RNA, 35 nt, Artificial Sequence
  Description of Artificial Sequence Synthetic oligonucleotide
accgagccgugugaccacagguuuagagcuaugcu

SEQ ID NO: 195 - RNA, 35 nt, Artificial Sequence
  Description of Artificial Sequence Synthetic oligonucleotide
ucuguccucaccgacauaauguuuagagcuaugcu

SEQ ID NO: 196 - RNA, 35 nt, Artificial Sequence
  Description of Artificial Sequence Synthetic oligonucleotide
aggugaagcagccuucucguguuuagagcuaugcu

SEQ ID NO: 197 - RNA, 35 nt, Artificial Sequence
  Description of Artificial Sequence Synthetic oligonucleotide
ucugacugacucggugccagguuuagagcuaugcu

SEQ ID NO: 198 - RNA, 35 nt, Artificial Sequence
  Description of Artificial Sequence Synthetic oligonucleotide
uucugacugacucggugccaguuuagagcuaugcu

SEQ ID NO: 199 - RNA, 35 nt, Artificial Sequence
  Description of Artificial Sequence Synthetic oligonucleotide
acuguggucacacggcucggguuuagagcuaugcu

SEQ ID NO: 200 - RNA, 35 nt, Artificial Sequence
  Description of Artificial Sequence Synthetic oligonucleotide
uucccuaagaaggucugcccguuuagagcuaugcu

SEQ ID NO: 201 - RNA, 35 nt, Artificial Sequence
  Description of Artificial Sequence Synthetic oligonucleotide
gucuguccucaccgacauaaguuuagagcuaugcu

SEQ ID NO: 202 - RNA, 35 nt, Artificial Sequence
  Description of Artificial Sequence Synthetic oligonucleotide
ccucaccgacauaaugggcuguuuagagcuaugcu

SEQ ID NO: 203 - RNA, 35 nt, Artificial Sequence
  Description of Artificial Sequence Synthetic oligonucleotide
ggcacguagacccggucccaguuuagagcuaugcu

SEQ ID NO: 204 - RNA, 100 nt, Artificial Sequence
  Description of Artificial Sequence Synthetic polynucleotide
cuucacgguucugagucuugguuuuagagcuagaaauagcaaguuaaaauaaggcuaguccguuaucaacuugaaaaaguggcaccgagucggugcuuuu

SEQ ID NO: 205 - RNA, 100 nt, Artificial Sequence
  Description of Artificial Sequence Synthetic polynucleotide
accgagccgugugaccacagguuuuagagcuagaaauagcaaguuaaaauaaggcuaguccguuaucaacuugaaaaaguggcaccgagucggugcuuuu

SEQ ID NO: 206 - RNA, 100 nt, Artificial Sequence
  Description of Artificial Sequence Synthetic polynucleotide
ucuguccucaccgacauaauguuuuagagcuagaaauagcaaguuaaaauaaggcuaguccguuaucaacuugaaaaaguggcaccgagucggugcuuuu

SEQ ID NO: 207 - RNA, 100 nt, Artificial Sequence
  Description of Artificial Sequence Synthetic polynucleotide
aggugaagcagccuucucguguuuuagagcuagaaauagcaaguuaaaauaaggcuaguccguuaucaacuugaaaaaguggcaccgagucggugcuuuu

SEQ ID NO: 208 - RNA, 100 nt, Artificial Sequence
  Description of Artificial Sequence Synthetic polynucleotide
ucugacugacucggugccagguuuuagagcuagaaauagcaaguuaaaauaaggcuaguccguuaucaacuugaaaaaguggcaccgagucggugcuuuu

SEQ ID NO: 209 - RNA, 100 nt, Artificial Sequence
  Description of Artificial Sequence Synthetic polynucleotide
uucugacugacucggugccaguuuuagagcuagaaauagcaaguuaaaauaaggcuaguccguuaucaacuugaaaaaguggcaccgagucggugcuuuu

SEQ ID NO: 210 - RNA, 100 nt, Artificial Sequence
  Description of Artificial Sequence Synthetic polynucleotide
acuguggucacacggcucggguuuuagagcuagaaauagcaaguuaaaauaaggcuaguccguuaucaacuugaaaaaguggcaccgagucggugcuuuu

SEQ ID NO: 211 - RNA, 100 nt, Artificial Sequence
  Description of Artificial Sequence Synthetic polynucleotide
uucccuaagaaggucugcccguuuuagagcuagaaauagcaaguuaaaauaaggcuaguccguuaucaacuugaaaaaguggcaccgagucggugcuuuu

SEQ ID NO: 212 - RNA, 100 nt, Artificial Sequence
  Description of Artificial Sequence Synthetic polynucleotide
gucuguccucaccgacauaaguuuuagagcuagaaauagcaaguuaaaauaaggcuaguccguuaucaacuugaaaaaguggcaccgagucggugcuuuu

SEQ ID NO: 213 - RNA, 100 nt, Artificial Sequence
  Description of Artificial Sequence Synthetic polynucleotide
ccucaccgacauaaugggcuguuuuagagcuagaaauagcaaguuaaaauaaggcuaguccguuaucaacuugaaaaaguggcaccgagucggugcuuuu

SEQ ID NO: 214 - RNA, 100 nt, Artificial Sequence
  Description of Artificial Sequence Synthetic polynucleotide
ggcacguagacccggucccaguuuuagagcuagaaauagcaaguuaaaauaaggcuaguccguuaucaacuugaaaaaguggcaccgagucggugcuuuu

SEQ ID NO: 215 - RNA, 20 nt, Artificial Sequence
  Description of Artificial Sequence Synthetic oligonucleotide
uauuaaugguccaucacagc

SEQ ID NO: 216 - RNA, 20 nt, Artificial Sequence
  Description of Artificial Sequence Synthetic oligonucleotide
ggaagcacaauucacguucc

SEQ ID NO: 217 - RNA, 20 nt, Artificial Sequence
  Description of Artificial Sequence Synthetic oligonucleotide
cucacugguauaaaccccug

SEQ ID NO: 218 - RNA, 20 nt, Artificial Sequence
  Description of Artificial Sequence Synthetic oligonucleotide
gcacaauucacguuccuggc

SEQ ID NO: 219 - RNA, 20 nt, Artificial Sequence
  Description of Artificial Sequence Synthetic oligonucleotide
agggaccacacgagcagcaa

SEQ ID NO: 220 - RNA, 20 nt, Artificial Sequence
  Description of Artificial Sequence Synthetic oligonucleotide
ggguuuauaccagugaggac

SEQ ID NO: 221 - RNA, 20 nt, Artificial Sequence
  Description of Artificial Sequence Synthetic oligonucleotide
ucugacaaggccucccaugc

SEQ ID NO: 222 - RNA, 20 nt, Artificial Sequence
  Description of Artificial Sequence Synthetic oligonucleotide
acgugaauugugcuuccuga

SEQ ID NO: 223 - RNA, 20 nt, Artificial Sequence
  Description of Artificial Sequence Synthetic oligonucleotide
acaauucacguuccuggcag

SEQ ID NO: 224 - RNA, 20 nt, Artificial Sequence
  Description of Artificial Sequence Synthetic oligonucleotide
ggaacgcauuuccuaacaug

SEQ ID NO: 225 - RNA, 20 nt, Artificial Sequence
  Description of Artificial Sequence Synthetic oligonucleotide
auugagagaccuugacuggc

SEQ ID NO: 226 - RNA, 20 nt, Artificial Sequence
  Description of Artificial Sequence Synthetic oligonucleotide
cuguucucauacauguccac

SEQ ID NO: 227 - RNA, 20 nt, Artificial Sequence
  Description of Artificial Sequence Synthetic oligonucleotide
cacaauucacguuccuggca

SEQ ID NO: 228 - RNA, 20 nt, Artificial Sequence
  Description of Artificial Sequence Synthetic oligonucleotide
caugaggcguguuuuauuaa

SEQ ID NO: 229 - RNA, 20 nt, Artificial Sequence
  Description of Artificial Sequence Synthetic oligonucleotide
ccuugacuggcuggccaugu

SEQ ID NO: 230 - RNA, 20 nt, Artificial Sequence
  Description of Artificial Sequence Synthetic oligonucleotide
ucuggcagcaagcacuaugc

SEQ ID NO: 231 - RNA, 20 nt, Artificial Sequence
  Description of Artificial Sequence Synthetic oligonucleotide
aaacuaaugccagauacauc

SEQ ID NO: 232 - RNA, 35 nt, Artificial Sequence
  Description of Artificial Sequence Synthetic oligonucleotide
uauuaaugguccaucacagcguuuagagcuaugcu

SEQ ID NO: 233 - RNA, 35 nt, Artificial Sequence
  Description of Artificial Sequence Synthetic oligonucleotide
ggaagcacaauucacguuccguuuagagcuaugcu

SEQ ID NO: 234 - RNA, 35 nt, Artificial Sequence
  Description of Artificial Sequence Synthetic oligonucleotide
cucacugguauaaaccccugguuuagagcuaugcu

SEQ ID NO: 235 - RNA, 35 nt, Artificial Sequence
  Description of Artificial Sequence Synthetic oligonucleotide
gcacaauucacguuccuggcguuuagagcuaugcu

SEQ ID NO: 236 - RNA, 35 nt, Artificial Sequence
  Description of Artificial Sequence Synthetic oligonucleotide
agggaccacacgagcagcaaguuuagagcuaugcu

SEQ ID NO: 237 - RNA, 35 nt, Artificial Sequence
  Description of Artificial Sequence Synthetic oligonucleotide
ggguuuauaccagugaggacguuuagagcuaugcu

SEQ ID NO: 238 - RNA, 35 nt, Artificial Sequence
  Description of Artificial Sequence Synthetic oligonucleotide
ucugacaaggccucccaugcguuuagagcuaugcu

SEQ ID NO: 239 - RNA, 35 nt, Artificial Sequence
  Description of Artificial Sequence Synthetic oligonucleotide
acgugaauugugcuuccugaguuuagagcuaugcu

SEQ ID NO: 240 - RNA, 35 nt, Artificial Sequence
  Description of Artificial Sequence Synthetic oligonucleotide
acaauucacguuccuggcagguuuagagcuaugcu

SEQ ID NO: 241 - RNA, 35 nt, Artificial Sequence
  Description of Artificial Sequence Synthetic oligonucleotide
ggaacgcauuuccuaacaugguuuagagcuaugcu

SEQ ID NO: 242 - RNA, 35 nt, Artificial Sequence
  Description of Artificial Sequence Synthetic oligonucleotide
auugagagaccuugacuggcguuuagagcuaugcu

SEQ ID NO: 243 - RNA, 35 nt, Artificial Sequence
  Description of Artificial Sequence Synthetic oligonucleotide
cuguucucauacauguccacguuuagagcuaugcu

SEQ ID NO: 244 - RNA, 35 nt, Artificial Sequence
  Description of Artificial Sequence Synthetic oligonucleotide
cacaauucacguuccuggcaguuuagagcuaugcu

SEQ ID NO: 245 - RNA, 35 nt, Artificial Sequence
  Description of Artificial Sequence Synthetic oligonucleotide
caugaggcguguuuuauuaaguuuagagcuaugcu

SEQ ID NO: 246 - RNA, 35 nt, Artificial Sequence
  Description of Artificial Sequence Synthetic oligonucleotide
ccuugacuggcuggccauguguuuagagcuaugcu

SEQ ID NO: 247 - RNA, 35 nt, Artificial Sequence
  Description of Artificial Sequence Synthetic oligonucleotide
ucuggcagcaagcacuaugcguuuagagcuaugcu

SEQ ID NO: 248 - RNA, 35 nt, Artificial Sequence
  Description of Artificial Sequence Synthetic oligonucleotide
aaacuaaugccagauacaucguuuagagcuaugcu

SEQ ID NO: 249 - RNA, 100 nt, Artificial Sequence
  Description of Artificial Sequence Synthetic polynucleotide
uauuaaugguccaucacagcguuuuagagcuagaaauagcaaguuaaaauaaggcuaguccguuaucaacuugaaaaaguggcaccgagucggugcuuuu

SEQ ID NO: 250 - RNA, 100 nt, Artificial Sequence
  Description of Artificial Sequence Synthetic polynucleotide
ggaagcacaauucacguuccguuuuagagcuagaaauagcaaguuaaaauaaggcuaguccguuaucaacuugaaaaaguggcaccgagucggugcuuuu

SEQ ID NO: 251 - RNA, 100 nt, Artificial Sequence
  Description of Artificial Sequence Synthetic polynucleotide
cucacugguauaaaccccugguuuuagagcuagaaauagcaaguuaaaauaaggcuaguccguuaucaacuugaaaaaguggcaccgagucggugcuuuu

SEQ ID NO: 252 - RNA, 100 nt, Artificial Sequence
  Description of Artificial Sequence Synthetic polynucleotide
gcacaauucacguuccuggcguuuuagagcuagaaauagcaaguuaaaauaaggcuaguccguuaucaacuugaaaaaguggcaccgagucggugcuuuu

SEQ ID NO: 253 - RNA, 100 nt, Artificial Sequence
  Description of Artificial Sequence Synthetic polynucleotide
agggaccacacgagcagcaaguuuuagagcuagaaauagcaaguuaaaauaaggcuaguccguuaucaacuugaaaaaguggcaccgagucggugcuuuu

SEQ ID NO: 254 - RNA, 100 nt, Artificial Sequence
  Description of Artificial Sequence Synthetic polynucleotide
ggguuuauaccagugaggacguuuuagagcuagaaauagcaaguuaaaauaaggcuaguccguuaucaacuugaaaaaguggcaccgagucggugcuuuu

SEQ ID NO: 255 - RNA, 100 nt, Artificial Sequence
  Description of Artificial Sequence Synthetic polynucleotide
ucugacaaggccucccaugcguuuuagagcuagaaauagcaaguuaaaauaaggcuaguccguuaucaacuugaaaaaguggcaccgagucggugcuuuu

SEQ ID NO: 256 - RNA, 100 nt, Artificial Sequence
  Description of Artificial Sequence Synthetic polynucleotide
acgugaauugugcuuccugaguuuuagagcuagaaauagcaaguuaaaauaaggcuaguccguuaucaacuugaaaaaguggcaccgagucggugcuuuu

SEQ ID NO: 257 - RNA, 100 nt, Artificial Sequence
  Description of Artificial Sequence Synthetic polynucleotide
acaauucacguuccuggcagguuuuagagcuagaaauagcaaguuaaaauaaggcuaguccguuaucaacuugaaaaaguggcaccgagucggugcuuuu

SEQ ID NO: 258 - RNA, 100 nt, Artificial Sequence
  Description of Artificial Sequence Synthetic polynucleotide
ggaacgcauuuccuaacaugguuuuagagcuagaaauagcaaguuaaaauaaggcuaguccguuaucaacuugaaaaaguggcaccgagucggugcuuuu

SEQ ID NO: 259 - RNA, 100 nt, Artificial Sequence
  Description of Artificial Sequence Synthetic polynucleotide
auugagagaccuugacuggcguuuuagagcuagaaauagcaaguuaaaauaaggcuaguccguuaucaacuugaaaaaguggcaccgagucggugcuuuu

SEQ ID NO: 260 - RNA, 100 nt, Artificial Sequence
  Description of Artificial Sequence Synthetic polynucleotide
cuguucucauacauguccacguuuuagagcuagaaauagcaaguuaaaauaaggcuaguccguuaucaacuugaaaaaguggcaccgagucggugcuuuu

SEQ ID NO: 261 - RNA, 100 nt, Artificial Sequence
  Description of Artificial Sequence Synthetic polynucleotide
cacaauucacguuccuggcaguuuuagagcuagaaauagcaaguuaaaauaaggcuaguccguuaucaacuugaaaaaguggcaccgagucggugcuuuu

SEQ ID NO: 262 - RNA, 100 nt, Artificial Sequence
  Description of Artificial Sequence Synthetic polynucleotide
caugaggcguguuuuauuaaguuuuagagcuagaaauagcaaguuaaaauaaggcuaguccguuaucaacuugaaaaaguggcaccgagucggugcuuuu

SEQ ID NO: 263 - RNA, 100 nt, Artificial Sequence
  Description of Artificial Sequence Synthetic polynucleotide
ccuugacuggcuggccauguguuuuagagcuagaaauagcaaguuaaaauaaggcuaguccguuaucaacuugaaaaaguggcaccgagucggugcuuuu

SEQ ID NO: 264 - RNA, 100 nt, Artificial Sequence
  Description of Artificial Sequence Synthetic polynucleotide
ucuggcagcaagcacuaugcguuuuagagcuagaaauagcaaguuaaaauaaggcuaguccguuaucaacuugaaaaaguggcaccgagucggugcuuuu

SEQ ID NO: 265 - RNA, 100 nt, Artificial Sequence
  Description of Artificial Sequence Synthetic polynucleotide
aaacuaaugccagauacaucguuuuagagcuagaaauagcaaguuaaaauaaggcuaguccguuaucaacuugaaaaaguggcaccgagucggugcuuuu

SEQ ID NO: 266 - RNA, 20 nt, Artificial Sequence
  Description of Artificial Sequence Synthetic oligonucleotide
auccuuaguagggucacaug

SEQ ID NO: 267 - RNA, 20 nt, Artificial Sequence
  Description of Artificial Sequence Synthetic oligonucleotide
ugugacccuacuaaggaugc

SEQ ID NO: 268 - RNA, 20 nt, Artificial Sequence
  Description of Artificial Sequence Synthetic oligonucleotide
acacuccuccuuauaugguc

SEQ ID NO: 269 - RNA, 20 nt, Artificial Sequence
  Description of Artificial Sequence Synthetic oligonucleotide
acgugcugaggucuaacaga

SEQ ID NO: 270 - RNA, 20 nt, Artificial Sequence
  Description of Artificial Sequence Synthetic oligonucleotide
aaccacaugugacccuacua

SEQ ID NO: 271 - RNA, 20 nt, Artificial Sequence
  Description of Artificial Sequence Synthetic oligonucleotide
aagagccagcauccuuagua

SEQ ID NO: 272 - RNA, 20 nt, Artificial Sequence
  Description of Artificial Sequence Synthetic oligonucleotide
gcacgugucucugugguuag

SEQ ID NO: 273 - RNA, 20 nt, Artificial Sequence
  Description of Artificial Sequence Synthetic oligonucleotide
ucugugguuagaggaguccg

SEQ ID NO: 274 - RNA, 20 nt, Artificial Sequence
  Description of Artificial Sequence Synthetic oligonucleotide
gugguuagaggaguccgugg

SEQ ID NO: 275 - RNA, 20 nt, Artificial Sequence
  Description of Artificial Sequence Synthetic oligonucleotide
uugagacacuccuccuuaua

SEQ ID NO: 276 - RNA, 20 nt, Artificial Sequence
  Description of Artificial Sequence Synthetic oligonucleotide
cugugagugcucauccuguc

SEQ ID NO: 277 - RNA, 20 nt, Artificial Sequence
  Description of Artificial Sequence Synthetic oligonucleotide
ccauucacugaccacaccau

SEQ ID NO: 278 - RNA, 20 nt, Artificial Sequence
  Description of Artificial Sequence Synthetic oligonucleotide
gugcugaggucuaacagaug

SEQ ID NO: 279 - RNA, 20 nt, Artificial Sequence
  Description of Artificial Sequence Synthetic oligonucleotide
acacaaccagcaagacuagc

SEQ ID NO: 280 - RNA, 20 nt, Artificial Sequence
  Description of Artificial Sequence Synthetic oligonucleotide
ggacacauuucuuaccugac

SEQ ID NO: 281 - RNA, 20 nt, Artificial Sequence
  Description of Artificial Sequence Synthetic oligonucleotide
gaagagccagcauccuuagu

SEQ ID NO: 282 - RNA, 35 nt, Artificial Sequence
  Description of Artificial Sequence Synthetic oligonucleotide
auccuuaguagggucacaugguuuagagcuaugcu

SEQ ID NO: 283 - RNA, 35 nt, Artificial Sequence
  Description of Artificial Sequence Synthetic oligonucleotide
ugugacccuacuaaggaugcguuuagagcuaugcu

SEQ ID NO: 284 - RNA, 35 nt, Artificial Sequence
  Description of Artificial Sequence Synthetic oligonucleotide
acacuccuccuuauauggucguuuagagcuaugcu

SEQ ID NO: 285 - RNA, 35 nt, Artificial Sequence
  Description of Artificial Sequence Synthetic oligonucleotide
acgugcugaggucuaacagaguuuagagcuaugcu

SEQ ID NO: 286 - RNA, 35 nt, Artificial Sequence
  Description of Artificial Sequence Synthetic oligonucleotide
aaccacaugugacccuacuaguuuagagcuaugcu

SEQ ID NO: 287 - RNA, 35 nt, Artificial Sequence
  Description of Artificial Sequence Synthetic oligonucleotide
aagagccagcauccuuaguaguuuagagcuaugcu

SEQ ID NO: 288 - RNA, 35 nt, Artificial Sequence
  Description of Artificial Sequence Synthetic oligonucleotide
gcacgugucucugugguuagguuuagagcuaugcu

SEQ ID NO: 289 - RNA, 35 nt, Artificial Sequence
  Description of Artificial Sequence Synthetic oligonucleotide
ucugugguuagaggaguccgguuuagagcuaugcu

SEQ ID NO: 290 - RNA, 35 nt, Artificial Sequence
  Description of Artificial Sequence Synthetic oligonucleotide
gugguuagaggaguccguggguuuagagcuaugcu

SEQ ID NO: 291 - RNA, 35 nt, Artificial Sequence
  Description of Artificial Sequence Synthetic oligonucleotide
uugagacacuccuccuuauaguuuagagcuaugcu

SEQ ID NO: 292 - RNA, 35 nt, Artificial Sequence
  Description of Artificial Sequence Synthetic oligonucleotide
cugugagugcucauccugucguuuagagcuaugcu

SEQ ID NO: 293 - RNA, 35 nt, Artificial Sequence
  Description of Artificial Sequence Synthetic oligonucleotide
ccauucacugaccacaccauguuuagagcuaugcu

SEQ ID NO: 294 - RNA, 35 nt, Artificial Sequence
  Description of Artificial Sequence Synthetic oligonucleotide
gugcugaggucuaacagaugguuuagagcuaugcu

SEQ ID NO: 295 - RNA, 35 nt, Artificial Sequence
  Description of Artificial Sequence Synthetic oligonucleotide
acacaaccagcaagacuagcguuuagagcuaugcu

SEQ ID NO: 296 - RNA, 35 nt, Artificial Sequence
  Description of Artificial Sequence Synthetic oligonucleotide
ggacacauuucuuaccugacguuuagagcuaugcu

SEQ ID NO: 297 - RNA, 35 nt, Artificial Sequence
  Description of Artificial Sequence Synthetic oligonucleotide
gaagagccagcauccuuaguguuuagagcuaugcu

SEQ ID NO: 298 - RNA, 100 nt, Artificial Sequence
  Description of Artificial Sequence Synthetic polynucleotide
auccuuaguagggucacaugguuuuagagcuagaaauagcaaguuaaaauaaggcuaguccguuaucaacuugaaaaaguggcaccgagucggugcuuuu

SEQ ID NO: 299 - RNA, 100 nt, Artificial Sequence
  Description of Artificial Sequence Synthetic polynucleotide
ugugacccuacuaaggaugcguuuuagagcuagaaauagcaaguuaaaauaaggcuaguccguuaucaacuugaaaaaguggcaccgagucggugcuuuu

SEQ ID NO: 300 - RNA, 100 nt, Artificial Sequence
  Description of Artificial Sequence Synthetic polynucleotide
acacuccuccuuauauggucguuuuagagcuagaaauagcaaguuaaaauaaggcuaguccguuaucaacuugaaaaaguggcaccgagucggugcuuuu

SEQ ID NO: 301 - RNA, 100 nt, Artificial Sequence
  Description of Artificial Sequence Synthetic polynucleotide
acgugcugaggucuaacagaguuuuagagcuagaaauagcaaguuaaaauaaggcuaguccguuaucaacuugaaaaaguggcaccgagucggugcuuuu

SEQ ID NO: 302 - RNA, 100 nt, Artificial Sequence
  Description of Artificial Sequence Synthetic polynucleotide
aaccacaugugacccuacuaguuuuagagcuagaaauagcaaguuaaaauaaggcuaguccguuaucaacuugaaaaaguggcaccgagucggugcuuuu

SEQ ID NO: 303 - RNA, 100 nt, Artificial Sequence
  Description of Artificial Sequence Synthetic polynucleotide
aagagccagcauccuuaguaguuuuagagcuagaaauagcaaguuaaaauaaggcuaguccguuaucaacuugaaaaaguggcaccgagucggugcuuuu

SEQ ID NO: 304 - RNA, 100 nt, Artificial Sequence
  Description of Artificial Sequence Synthetic polynucleotide
gcacgugucucugugguuagguuuuagagcuagaaauagcaaguuaaaauaaggcuaguccguuaucaacuugaaaaaguggcaccgagucggugcuuuu

SEQ ID NO: 305 - RNA, 100 nt, Artificial Sequence
  Description of Artificial Sequence Synthetic polynucleotide
ucugugguuagaggaguccgguuuuagagcuagaaauagcaaguuaaaauaaggcuaguccguuaucaacuugaaaaaguggcaccgagucggugcuuuu

SEQ ID NO: 306 - RNA, 100 nt, Artificial Sequence
  Description of Artificial Sequence Synthetic polynucleotide
gugguuagaggaguccguggguuuuagagcuagaaauagcaaguuaaaauaaggcuaguccguuaucaacuugaaaaaguggcaccgagucggugcuuuu

SEQ ID NO: 307 - RNA, 100 nt, Artificial Sequence
  Description of Artificial Sequence Synthetic polynucleotide
uugagacacuccuccuuauaguuuuagagcuagaaauagcaaguuaaaauaaggcuaguccguuaucaacuugaaaaaguggcaccgagucggugcuuuu

SEQ ID NO: 308 - RNA, 100 nt, Artificial Sequence
  Description of Artificial Sequence Synthetic polynucleotide
cugugagugcucauccugucguuuuagagcuagaaauagcaaguuaaaauaaggcuaguccguuaucaacuugaaaaaguggcaccgagucggugcuuuu

SEQ ID NO: 309 - RNA, 100 nt, Artificial Sequence
  Description of Artificial Sequence Synthetic polynucleotide
ccauucacugaccacaccauguuuuagagcuagaaauagcaaguuaaaauaaggcuaguccguuaucaacuugaaaaaguggcaccgagucggugcuuuu

SEQ ID NO: 310 - RNA, 100 nt, Artificial Sequence
  Description of Artificial Sequence Synthetic polynucleotide
gugcugaggucuaacagaugguuuuagagcuagaaauagcaaguuaaaauaaggcuaguccguuaucaacuugaaaaaguggcaccgagucggugcuuuu

SEQ ID NO: 311 - RNA, 100 nt, Artificial Sequence
  Description of Artificial Sequence Synthetic polynucleotide
acacaaccagcaagacuagcguuuuagagcuagaaauagcaaguuaaaauaaggcuaguccguuaucaacuugaaaaaguggcaccgagucggugcuuuu

SEQ ID NO: 312 - RNA, 100 nt, Artificial Sequence
  Description of Artificial Sequence Synthetic polynucleotide
ggacacauuucuuaccugacguuuuagagcuagaaauagcaaguuaaaauaaggcuaguccguuaucaacuugaaaaaguggcaccgagucggugcuuuu

SEQ ID NO: 313 - RNA, 100 nt, Artificial Sequence
  Description of Artificial Sequence Synthetic polynucleotide
gaagagccagcauccuuaguguuuuagagcuagaaauagcaaguuaaaauaaggcuaguccguuaucaacuugaaaaaguggcaccgagucggugcuuuu

SEQ ID NO: 314 - RNA, 20 nt, Artificial Sequence
  Description of Artificial Sequence Synthetic oligonucleotide
gaguauucuuguaagacacg

SEQ ID NO: 315 - RNA, 20 nt, Artificial Sequence
  Description of Artificial Sequence Synthetic oligonucleotide
gguguagggaaccaacacag

SEQ ID NO: 316 - RNA, 20 nt, Artificial Sequence
  Description of Artificial Sequence Synthetic oligonucleotide
ugaugaggugagcacacacg

SEQ ID NO: 317 - RNA, 20 nt, Artificial Sequence
  Description of Artificial Sequence Synthetic oligonucleotide
cucggaguuuuucacuggag

SEQ ID NO: 318 - RNA, 20 nt, Artificial Sequence
  Description of Artificial Sequence Synthetic oligonucleotide
ucguuguuguccucuacuuu

SEQ ID NO: 319 - RNA, 20 nt, Artificial Sequence
  Description of Artificial Sequence Synthetic oligonucleotide
ggcuuuaucaaagugauccc

SEQ ID NO: 320 - RNA, 20 nt, Artificial Sequence
  Description of Artificial Sequence Synthetic oligonucleotide
aagcugauaugcaggaaccc

SEQ ID NO: 321 - RNA, 20 nt, Artificial Sequence
  Description of Artificial Sequence Synthetic oligonucleotide
gcaaguuuuaggcuaugucc

SEQ ID NO: 322 - RNA, 20 nt, Artificial Sequence
  Description of Artificial Sequence Synthetic oligonucleotide
gagcacaacucugagagggu

SEQ ID NO: 323 - RNA, 20 nt, Artificial Sequence
  Description of Artificial Sequence Synthetic oligonucleotide
aaguucucggaguuuuucac

SEQ ID NO: 324 - RNA, 35 nt, Artificial Sequence
  Description of Artificial Sequence Synthetic oligonucleotide
gaguauucuuguaagacacgguuuagagcuaugcu

SEQ ID NO: 325 - RNA, 35 nt, Artificial Sequence
  Description of Artificial Sequence Synthetic oligonucleotide
gguguagggaaccaacacagguuuagagcuaugcu

SEQ ID NO: 326 - RNA, 35 nt, Artificial Sequence
  Description of Artificial Sequence Synthetic oligonucleotide
ugaugaggugagcacacacgguuuagagcuaugcu

SEQ ID NO: 327 - RNA, 35 nt, Artificial Sequence
  Description of Artificial Sequence Synthetic oligonucleotide
cucggaguuuuucacuggagguuuagagcuaugcu

SEQ ID NO: 328 - RNA, 35 nt, Artificial Sequence
  Description of Artificial Sequence Synthetic oligonucleotide
ucguuguuguccucuacuuuguuuagagcuaugcu

SEQ ID NO: 329 - RNA, 35 nt, Artificial Sequence
  Description of Artificial Sequence Synthetic oligonucleotide
ggcuuuaucaaagugaucccguuuagagcuaugcu

SEQ ID NO: 330 - RNA, 35 nt, Artificial Sequence
  Description of Artificial Sequence Synthetic oligonucleotide
aagcugauaugcaggaacccguuuagagcuaugcu

SEQ ID NO: 331 - RNA, 35 nt, Artificial Sequence
  Description of Artificial Sequence Synthetic oligonucleotide
gcaaguuuuaggcuauguccguuuagagcuaugcu

SEQ ID NO: 332 - RNA, 35 nt, Artificial Sequence
  Description of Artificial Sequence Synthetic oligonucleotide
gagcacaacucugagaggguguuuagagcuaugcu

SEQ ID NO: 333 - RNA, 35 nt, Artificial Sequence
  Description of Artificial Sequence Synthetic oligonucleotide
aaguucucggaguuuuucacguuuagagcuaugcu

SEQ ID NO: 334 - RNA, 100 nt, Artificial Sequence
  Description of Artificial Sequence Synthetic polynucleotide
gaguauucuuguaagacacgguuuuagagcuagaaauagcaaguuaaaauaaggcuaguccguuaucaacuugaaaaaguggcaccgagucggugcuuuu

SEQ ID NO: 335 - RNA, 100 nt, Artificial Sequence
  Description of Artificial Sequence Synthetic polynucleotide
gguguagggaaccaacacagguuuuagagcuagaaauagcaaguuaaaauaaggcuaguccguuaucaacuugaaaaaguggcaccgagucggugcuuuu

SEQ ID NO: 336 - RNA, 100 nt, Artificial Sequence
  Description of Artificial Sequence Synthetic polynucleotide
ugaugaggugagcacacacgguuuuagagcuagaaauagcaaguuaaaauaaggcuaguccguuaucaacuugaaaaaguggcaccgagucggugcuuuu

SEQ ID NO: 337 - RNA, 100 nt, Artificial Sequence
  Description of Artificial Sequence Synthetic polynucleotide
cucggaguuuuucacuggagguuuuagagcuagaaauagcaaguuaaaauaaggcuaguccguuaucaacuugaaaaaguggcaccgagucggugcuuuu

SEQ ID NO: 338 - RNA, 100 nt, Artificial Sequence
  Description of Artificial Sequence Synthetic polynucleotide
ucguuguuguccucuacuuuguuuuagagcuagaaauagcaaguuaaaauaaggcuaguccguuaucaacuugaaaaaguggcaccgagucggugcuuuu

SEQ ID NO: 339 - RNA, 100 nt, Artificial Sequence
  Description of Artificial Sequence Synthetic polynucleotide
ggcuuuaucaaagugaucccguuuuagagcuagaaauagcaaguuaaaauaaggcuaguccguuaucaacuugaaaaaguggcaccgagucggugcuuuu

SEQ ID NO: 340 - RNA, 100 nt, Artificial Sequence
  Description of Artificial Sequence Synthetic polynucleotide
aagcugauaugcaggaacccguuuuagagcuagaaauagcaaguuaaaauaaggcuaguccguuaucaacuugaaaaaguggcaccgagucggugcuuuu

SEQ ID NO: 341 - RNA, 100 nt, Artificial Sequence
  Description of Artificial Sequence Synthetic polynucleotide
gcaaguuuuaggcuauguccguuuuagagcuagaaauagcaaguuaaaauaaggcuaguccguuaucaacuugaaaaaguggcaccgagucggugcuuuu

SEQ ID NO: 342 - RNA, 100 nt, Artificial Sequence
  Description of Artificial Sequence Synthetic polynucleotide
gagcacaacucugagaggguguuuuagagcuagaaauagcaaguuaaaauaaggcuaguccguuaucaacuugaaaaaguggcaccgagucggugcuuuu

SEQ ID NO: 343 - RNA, 100 nt, Artificial Sequence
  Description of Artificial Sequence Synthetic polynucleotide
aaguucucggaguuuuucacguuuuagagcuagaaauagcaaguuaaaauaaggcuaguccguuaucaacuugaaaaaguggcaccgagucggugcuuuu

SEQ ID NO: 344 - RNA, 20 nt, Artificial Sequence
  Description of Artificial Sequence Synthetic oligonucleotide
aacauuuucaauccgaugag

SEQ ID NO: 345 - RNA, 20 nt, Artificial Sequence
  Description of Artificial Sequence Synthetic oligonucleotide
gaaacauuuucaauccgaug

SEQ ID NO: 346 - RNA, 35 nt, Artificial Sequence
  Description of Artificial Sequence Synthetic oligonucleotide
aacauuuucaauccgaugagguuuagagcuaugcu

SEQ ID NO: 347 - RNA, 35 nt, Artificial Sequence
  Description of Artificial Sequence Synthetic oligonucleotide
gaaacauuuucaauccgaugguuuagagcuaugcu

SEQ ID NO: 348 - RNA, 100 nt, Artificial Sequence
  Description of Artificial Sequence Synthetic polynucleotide
aacauuuucaauccgaugagguuuuagagcuagaaauagcaaguuaaaauaaggcuaguccguuaucaacuugaaaaaguggcaccgagucggugcuuuu

SEQ ID NO: 349 - RNA, 100 nt, Artificial Sequence
  Description of Artificial Sequence Synthetic polynucleotide
gaaacauuuucaauccgaugguuuuagagcuagaaauagcaaguuaaaauaaggcuaguccguuaucaacuugaaaaaguggcaccgagucggugcuuuu

SEQ ID NO: 350 - RNA, 20 nt, Artificial Sequence
  Description of Artificial Sequence Synthetic oligonucleotide
acggacacuaccaaggagcg

SEQ ID NO: 351 - RNA, 20 nt, Artificial Sequence
  Description of Artificial Sequence Synthetic oligonucleotide
acaugaacgaggccaagcgg

SEQ ID NO: 352 - RNA, 20 nt, Artificial Sequence
  Description of Artificial Sequence Synthetic oligonucleotide
gacacuaccaaggagcgcgg

SEQ ID NO: 353 - RNA, 20 nt, Artificial Sequence
  Description of Artificial Sequence Synthetic oligonucleotide
uuucagucgggacaugaacg

SEQ ID NO: 354 - RNA, 20 nt, Artificial Sequence
  Description of Artificial Sequence Synthetic oligonucleotide
acacuaccaaggagcgcggc

SEQ ID NO: 355 - RNA, 20 nt, Artificial Sequence
  Description of Artificial Sequence Synthetic oligonucleotide
ugagagguagcggcuuacgu

SEQ ID NO: 356 - RNA, 20 nt, Artificial Sequence
  Description of Artificial Sequence Synthetic oligonucleotide
aaugggcuucacgaaggugc

SEQ ID NO: 357 - RNA, 20 nt, Artificial Sequence
  Description of Artificial Sequence Synthetic oligonucleotide
gaaugucccugucuacgaug

SEQ ID NO: 358 - RNA, 20 nt, Artificial Sequence
  Description of Artificial Sequence Synthetic oligonucleotide
caugaacgaggccaagcgga

SEQ ID NO: 359 - RNA, 20 nt, Artificial Sequence
  Description of Artificial Sequence Synthetic oligonucleotide
cgacagaugguguaguccaa

SEQ ID NO: 360 - RNA, 20 nt, Artificial Sequence
  Description of Artificial Sequence Synthetic oligonucleotide
cuugaagaaccuccucgugg

SEQ ID NO: 361 - RNA, 20 nt, Artificial Sequence
  Description of Artificial Sequence Synthetic oligonucleotide
gaugcuuuggcaagauggcg

SEQ ID NO: 362 - RNA, 20 nt, Artificial Sequence
  Description of Artificial Sequence Synthetic oligonucleotide
uugaagaaccuccucguggc

SEQ ID NO: 363 - RNA, 20 nt, Artificial Sequence
  Description of Artificial Sequence Synthetic oligonucleotide
ucgugaagcccauuuucagu

SEQ ID NO: 364 - RNA, 20 nt, Artificial Sequence
  Description of Artificial Sequence Synthetic oligonucleotide
acugaaaaugggcuucacga

SEQ ID NO: 365 - RNA, 20 nt, Artificial Sequence
  Description of Artificial Sequence Synthetic oligonucleotide
cacgacccagcgaccuccug

SEQ ID NO: 366 - RNA, 20 nt, Artificial Sequence
  Description of Artificial Sequence Synthetic oligonucleotide
uucugagugucucucuucgc

SEQ ID NO: 367 - RNA, 20 nt, Artificial Sequence
  Description of Artificial Sequence Synthetic oligonucleotide
ugacucugaggcucucggau

SEQ ID NO: 368 - RNA, 20 nt, Artificial Sequence
  Description of Artificial Sequence Synthetic oligonucleotide
aggugccgaacacuggugag

SEQ ID NO: 369 - RNA, 20 nt, Artificial Sequence
  Description of Artificial Sequence Synthetic oligonucleotide
ggaccccgagguaacugcug

SEQ ID NO: 370 - RNA, 20 nt, Artificial Sequence
  Description of Artificial Sequence Synthetic oligonucleotide
ggccuugaagaaccuccucg

SEQ ID NO: 371 - RNA, 20 nt, Artificial Sequence
  Description of Artificial Sequence Synthetic oligonucleotide
cccaaguuggugaccucagc

SEQ ID NO: 372 - RNA, 20 nt, Artificial Sequence
  Description of Artificial Sequence Synthetic oligonucleotide
ccagcugaggucaccaacuu

SEQ ID NO: 373 - RNA, 35 nt, Artificial Sequence; identical to SEQ ID NO: 136
  Description of Artificial Sequence Synthetic oligonucleotide

SEQ ID NO: 374 - RNA, 35 nt, Artificial Sequence; identical to SEQ ID NO: 129
  Description of Artificial Sequence Synthetic oligonucleotide

SEQ ID NO: 375 - RNA, 35 nt, Artificial Sequence; identical to SEQ ID NO: 137
  Description of Artificial Sequence Synthetic oligonucleotide

SEQ ID NO: 376 - RNA, 35 nt, Artificial Sequence; identical to SEQ ID NO: 138
  Description of Artificial Sequence Synthetic oligonucleotide

SEQ ID NO: 377 - RNA, 35 nt, Artificial Sequence; identical to SEQ ID NO: 139
  Description of Artificial Sequence Synthetic oligonucleotide

SEQ ID NO: 378 - RNA, 35 nt, Artificial Sequence; identical to SEQ ID NO: 141
  Description of Artificial Sequence Synthetic oligonucleotide

SEQ ID NO: 379 - RNA, 35 nt, Artificial Sequence; identical to SEQ ID NO: 133
  Description of Artificial Sequence Synthetic oligonucleotide

SEQ ID NO: 380 - RNA, 35 nt, Artificial Sequence; identical to SEQ ID NO: 134
  Description of Artificial Sequence Synthetic oligonucleotide

SEQ ID NO: 381 - RNA, 35 nt, Artificial Sequence; identical to SEQ ID NO: 130
  Description of Artificial Sequence Synthetic oligonucleotide

SEQ ID NO: 382 - RNA, 35 nt, Artificial Sequence; identical to SEQ ID NO: 131
  Description of Artificial Sequence Synthetic oligonucleotide

SEQ ID NO: 383 - RNA, 35 nt, Artificial Sequence; identical to SEQ ID NO: 132
  Description of Artificial Sequence Synthetic oligonucleotide

SEQ ID NO: 384 - RNA, 35 nt, Artificial Sequence; identical to SEQ ID NO: 127
  Description of Artificial Sequence Synthetic oligonucleotide

SEQ ID NO: 385 - RNA, 35 nt, Artificial Sequence; identical to SEQ ID NO: 128
  Description of Artificial Sequence Synthetic oligonucleotide

SEQ ID NO: 386 - RNA, 35 nt, Artificial Sequence; identical to SEQ ID NO: 124
  Description of Artificial Sequence Synthetic oligonucleotide

SEQ ID NO: 387 - RNA, 35 nt, Artificial Sequence; identical to SEQ ID NO: 125
  Description of Artificial Sequence Synthetic oligonucleotide

SEQ ID NO: 388 - RNA, 35 nt, Artificial Sequence; identical to SEQ ID NO: 126
  Description of Artificial Sequence Synthetic oligonucleotide

SEQ ID NO: 389 - RNA, 35 nt, Artificial Sequence
  Description of Artificial Sequence Synthetic oligonucleotide
uucugagugucucucuucgcguuuagagcuaugcu

SEQ ID NO: 390 - RNA, 35 nt, Artificial Sequence; identical to SEQ ID NO: 123
  Description of Artificial Sequence Synthetic oligonucleotide

SEQ ID NO: 391 - RNA, 35 nt, Artificial Sequence; identical to SEQ ID NO: 120
  Description of Artificial Sequence Synthetic oligonucleotide

SEQ ID NO: 392 - RNA, 35 nt, Artificial Sequence; identical to SEQ ID NO: 121
  Description of Artificial Sequence Synthetic oligonucleotide

SEQ ID NO: 393 - RNA, 35 nt, Artificial Sequence; identical to SEQ ID NO: 122
  Description of Artificial Sequence Synthetic oligonucleotide

SEQ ID NO: 394 - RNA, 35 nt, Artificial Sequence; identical to SEQ ID NO: 118
  Description of Artificial Sequence Synthetic oligonucleotide

SEQ ID NO: 395 - RNA, 35 nt, Artificial Sequence; identical to SEQ ID NO: 119
  Description of Artificial Sequence Synthetic oligonucleotide

SEQ ID NO: 396 - RNA, 100 nt, Artificial Sequence; identical to SEQ ID NO: 3
  Description of Artificial Sequence Synthetic polynucleotide

SEQ ID NO: 397 - RNA, 100 nt, Artificial Sequence; identical to SEQ ID NO: 154
  Description of Artificial Sequence Synthetic polynucleotide

SEQ ID NO: 398 - RNA, 100 nt, Artificial Sequence; identical to SEQ ID NO: 162
  Description of Artificial Sequence Synthetic polynucleotide

SEQ ID NO: 399 - RNA, 100 nt, Artificial Sequence; identical to SEQ ID NO: 163
  Description of Artificial Sequence Synthetic polynucleotide

SEQ ID NO: 400 - RNA, 100 nt, Artificial Sequence; identical to SEQ ID NO: 164
  Description of Artificial Sequence Synthetic polynucleotide

SEQ ID NO: 401 - RNA, 100 nt, Artificial Sequence; identical to SEQ ID NO: 166
  Description of Artificial Sequence Synthetic polynucleotide

SEQ ID NO: 402 - RNA, 100 nt, Artificial Sequence; identical to SEQ ID NO: 158
  Description of Artificial Sequence Synthetic polynucleotide

SEQ ID NO: 403 - RNA, 100 nt, Artificial Sequence; identical to SEQ ID NO: 159
  Description of Artificial Sequence Synthetic polynucleotide

SEQ ID NO: 404 - RNA, 100 nt, Artificial Sequence; identical to SEQ ID NO: 155
  Description of Artificial Sequence Synthetic polynucleotide

SEQ ID NO: 405 - RNA, 100 nt, Artificial Sequence; identical to SEQ ID NO: 156
  Description of Artificial Sequence Synthetic polynucleotide

SEQ ID NO: 406 - RNA, 100 nt, Artificial Sequence; identical to SEQ ID NO: 4
  Description of Artificial Sequence Synthetic polynucleotide

SEQ ID NO: 407 - RNA, 100 nt, Artificial Sequence; identical to SEQ ID NO: 152
  Description of Artificial Sequence Synthetic polynucleotide

SEQ ID NO: 408 - RNA, 100 nt, Artificial Sequence; identical to SEQ ID NO: 153
  Description of Artificial Sequence Synthetic polynucleotide

SEQ ID NO: 409 - RNA, 100 nt, Artificial Sequence; identical to SEQ ID NO: 149
  Description of Artificial Sequence Synthetic polynucleotide

SEQ ID NO: 410 - RNA, 100 nt, Artificial Sequence; identical to SEQ ID NO: 150
  Description of Artificial Sequence Synthetic polynucleotide

SEQ ID NO: 411 - RNA, 100 nt, Artificial Sequence; identical to SEQ ID NO: 151
  Description of Artificial Sequence Synthetic polynucleotide

SEQ ID NO: 412 - RNA, 100 nt, Artificial Sequence
  Description of Artificial Sequence Synthetic polynucleotide
uucugagugucucucuucgcguuuuagagcuagaaauagcaaguuaaaauaaggcuaguccguuaucaacuugaaaaaguggcaccgagucggugcuuuu

SEQ ID NO: 413 - RNA, 100 nt, Artificial Sequence; identical to SEQ ID NO: 148
  Description of Artificial Sequence Synthetic polynucleotide

SEQ ID NO: 414 - RNA, 100 nt, Artificial Sequence; identical to SEQ ID NO: 145
  Description of Artificial Sequence Synthetic polynucleotide

SEQ ID NO: 415 - RNA, 100 nt, Artificial Sequence; identical to SEQ ID NO: 146
  Description of Artificial Sequence Synthetic polynucleotide

SEQ ID NO: 416 - RNA, 100 nt, Artificial Sequence; identical to SEQ ID NO: 147
  Description of Artificial Sequence Synthetic polynucleotide

SEQ ID NO: 417 - RNA, 100 nt, Artificial Sequence; identical to SEQ ID NO: 143
  Description of Artificial Sequence Synthetic polynucleotide

SEQ ID NO: 418 - RNA, 100 nt, Artificial Sequence; identical to SEQ ID NO: 144
  Description of Artificial Sequence Synthetic polynucleotide